(19) United States
(12) Patent Application Publication
(10) Pub. No.: US 2022/0339191 A1
(43) Pub. Date: Oct. 27, 2022

(54) INTERLEUKIN-27 PRODUCING B-CELLS AND USES THEREOF

(71) Applicant: The United States of America,as represented by the Secretary,Department of Health and Human Services, Bethesda, MD (US)

(72) Inventors: Charles E. Egwuagu, Silver Spring, MD (US); Jin Kyeong Choi, Busan (KR)

(73) Assignee: The United States of America,as represented by the Secretary,Department of Health and Human Services, Bethesda, MD (US)

(21) Appl. No.: 17/620,248

(22) PCT Filed: Jun. 18, 2020
(86) PCT No.: PCT/US2020/038368

§ 371 Date: Dec. 17, 2021

Related U.S. Application Data
(60) Provisional application No. 62/863,054, filed on Jun. 18, 2019.

(51) Int. Cl. A61K 35/17; C12N 5/0781; A61K 38/20

(52) U.S. Cl. CPC A61K 35/17; C12N 5/0635; A61K 38/20; C12N 2501/2327; C12N 2501/998; C12N 2502/1157; C12N 2502/1121

(57) ABSTRACT

The invention is directed to an isolated population of mammal cells comprising about 75% or higher B-1a regula e PBS-treated tory cells expressing the cell surface inhibitory receptors lympho-cyte-activation gene 3 (LAG-3), programmed cell death protein 1 (PD-1), and C-X-C chemokine receptor type 4 (CXCR4), and secreting interleukin-27 (IL-27). The invention is also directed to methods of preparing and using the cell population to suppress the immune system and/or to treat or prevent diseases.

### CROSS-REFERENCE TO RELATED APPLICATION

This patent application claims the benefit of co-pending U.S. Provisional Patent Application No. 62/863,054, filed Jun. 18, 2019, which is incorporated by reference in its entirety herein.

### STATEMENT REGARDING FEDERALLY SPONSORED RESEARCH OR DEVELOPMENT

This invention was made with Government support under project number Z01EY000350-18 by the National Eye Institute of the National Institutes of Health. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,692 Byte ASCII (Text) file named “749447_ST25.TXT,” created on Jun. 12, 2020.

### BACKGROUND OF THE INVENTION

Uveitis, age-related macular degeneration (AMD), graft-vs-host disease (GVHD), and multiple sclerosis (MS) are diseases that initiate or progress as a result of adverse immunological activity. These diseases can result in blindness, paralysis, and significant morbidity that impacts quality of life. Uveitis is comprised of a diverse group of potentially sight-threatening intraocular inflammatory diseases of infectious or autoimmune etiology, where autoreactive lymphocytes contribute to ocular pathology by attacking and damaging uveal tissue. Similarly, autoimmune processes contribute significantly to the progression of retinal degeneration associated with AMD, though the processes that initiate AMD have not been definitively identified. MS is caused in part by lymphocytes that attack and/or destroy myelinated neurons, thereby interfering with synaptic transmission and communication between neurons. In GVHD, the allogeneic transplant views the recipient's body as foreign, and the transplant attacks the body. Although steroids are effective therapy for uveitis or MS, serious adverse effects preclude their prolonged use. Similar to uveitis and MS, there may be adverse effects associated with the use of steroids and immunosuppressants to treat GVHD. Further, there currently is no effective cure for AMD, and current treatments are directed to the slowing of progressive retinal degeneration. Therefore, there remains an unmet need for safe and effective long-term therapies for the aforesaid diseases.

### BRIEF SUMMARY OF THE INVENTION

The invention provides an isolated population of mammal cells comprising about 75% or higher B-1a regulatory cells expressing cell surface inhibitory receptors lymphocyte-activation gene 3 (LAG-3), programmed cell death protein 1 (PD-1), and C-X-C chemokine receptor type 4 (CXCR4), and secreting interleukin-27 (IL-27).

The invention also provides methods of preparing the population of mammal cells of an embodiment of the invention, comprising (a) isolating cluster of differentiation 5 positive (CD5+) expressing cells from a sample of mammal peripheral lymphoid tissue, mammal cord blood, mammal peritoneal fluid, or mammal bone marrow using fluorescence-activated cell sorting (FACS) to provide isolated CD5+ expressing cells; (b) culturing the isolated CD5+ expressing cells in a cell culture media to provide cultured cells; (c) activating the cultured cells with a BCR (B cell receptor) or a TLR (Toll-like receptor) agonists to provide activated cells; and (d) exposing the activated cells to IL-27.

The invention further provides methods of suppressing the immune system of a mammal, the method comprising administering the population of mammal cells of an embodiment of the invention to a mammal.

The invention further provides methods of treating a mammal with graft-versus-host disease, the method comprising administering the population of mammal cells of an embodiment of the invention to a mammal with graft-versus-host disease.

The invention provides methods of preventing or reducing the severity of graft-versus-host disease in a mammal, the method comprising administering the population of mammal cells of an embodiment of the invention to a mammal before the mammal receives an allogeneic transplant.

The invention provides methods of preventing or reducing the severity of graft-versus-host disease in a mammal, the method comprising (a) mixing the population of mammal cells of an embodiment of the invention with a transplant material to form a transplant mixture, and (b) administering the transplant mixture to a mammal.

### BRIEF DESCRIPTION OF THE SEVERAL VIEWS OF THE DRAWINGS

FIG. 1 is a set of confocal microscopy images showing sorted CD19⁺ B cells from C57BL/6 mice. The cells activated in vitro for 48 h by stimulation with lipopolysaccharides (LPS) or anti-CD40/anti-IgM antibodies (BCR). The cells were incubated with fluorescence labelled anti-p28 or anti-Ebi3 antibody. The cells expressing IL-27 (co-expression of p28 and Ebi3) were detected by confocal microscopy (white arrows).

FIG. 2A is a set of flow cytometry plots showing sorted CD19+ B cells isolated from the peritoneal cavity or spleen of C57BL/6J mice activated in vitro for 48 h by stimulation with LPS or BCR. The plots show the percentage of B-1a and B2 cells expressing IL-27.

FIG. 2B is a bar graph showing the percentage of B-1a and B2 cells of FIG. 2A from the peritoneal cavity that express IL-27.

FIG. 2C is a bar graph showing the percentage of B-1a and B2 cells of FIG. 2A from the spleen that express IL-27.

FIG. 2D is a graph showing the results of analysis of the supernatants of the cultures of FIG. 2A by enzyme-linked immunosorbent assay (ELISA).

FIG. 3 is a set of flow cytometry plots showing sorted CD19+ B-cells from C57BL/6J mice activated in vitro for 48 h by stimulation with anti-CD40/anti-IgM antibodies (BCR) in the presence or absence of IL-27. The plots show the frequency of various cells in the culture. The numbers in the quadrants indicate the percentage of B cells expressing p28, Ebi3 or p28, and Ebi3 (IL-27).

FIG. 4 is a bar graph showing the quantification frequency of various cells in the culture shown in the plots of FIG. 3.

FIG. 5 is a graph that shows the results of NanoString RNA analysis (NanoString Technologies, Inc., Seattle, Wash.) of various cells in the culture shown in the plots of FIG. 3 showing that BCR/IL-27 synergistically upregulated expression of IL-27 subunit p28 and IL-27Ra and altered the pattern of chemokine receptors expression.

FIG. 6 is a set of images showing the results of immunofluorescence/confocal microscopy analysis of various cells in the culture shown in the plots of FIG. 3 showing that BCR/IL-27 synergistically upregulated expression of IL-27 subunit p28 and IL-27Rα and altered the pattern of chemokine receptors expression. The cells expressing IL-27 (co-expression of p28 and Ebi3) were detected by confocal microscopy (white arrows).

FIG. 7 is a graph showing sorted CD19⁺ B cells from wild type or IL-27RαKO mice activated in vitro for 48 h by stimulation with anti-CD40/anti-IgM antibodies (BCR) in the presence or absence of IL-27. B cells expressing p28, Ebi3, or p28 and Ebi3 (IL-27) were detected by intracellular cytokine assay and the bar chart shows the percentages of IL-27-producing B cells in the various cultures.

FIG. 8 is a graph showing the results of qPCR for expression of IL-27Rα in cells that were isolated from the peritoneal cavity and spleen of wild type mice and sorted into B-1a or B2 cells.

FIG. 9 shows CD19⁺ B cells isolated from human peripheral blood mononuclear cells (PBMC) of human volunteers that were activated with phorbol myristate acetate (PMA) in the presence of IL-27.

FIG. 10 is a graph showing the CD19⁺ B cells of FIG. 9 in the presence of IL-27.

FIG. 11 is a graph showing the frequency of human B cells expressing p28, Ebi3 or both p28 and Ebi3 (IL-27) after CD19⁺ B cells were isolated from PBMC of human volunteers and activated with PMA in the absence of IL-27.

FIG. 12 is a flow cytometry plots showing the frequency of the cells of FIG. 11 that express p28, Ebi3 or both p28 and Ebi3 (IL-27).

FIG. 13A is a bar graph showing the frequency of IL-27-producing B-la cells in the peritoneal cavity. C57BL/6J mice were injected (i.v) with LPS (50 μg/mouse) and frequency of IL-27-producing B-1a cells in the peritoneal cavity was assessed every day until day 4 post-injection. The B-1a cells were isolated at various time points from the peritoneal cavity and analyzed by intracellular cytokine staining assay.

FIG. 13B is a bar graph showing the frequency of IL-27-producing B2 cells in the peritoneal cavity. C57BL/6J mice were injected (i.v) with LPS (50 μg/mouse) and frequency of IL-27-producing B2 cells in the peritoneal cavity was assessed every day until day 4 post-injection. The B2 cells were isolated at various time points from the peritoneal cavity and analyzed by intracellular cytokine staining assay.

FIG. 14A is a bar graph showing the frequency of IL-27-producing B-1a cells in the spleen. C57BL/6J mice were injected (i.v) with LPS (50 μg/mouse) and frequency of IL-27-producing B-1a cells in the spleen was assessed every day until day 4 post-injection. The B-1a cells were isolated at various time points from the spleen and analyzed by intracellular cytokine staining assay.

FIG. 14B is a bar graph showing the frequency of IL-27-producing B2 cells in the spleen.

C57BL/6J mice were injected (i.v) with LPS (50 μg/mouse) and frequency of IL-27-producing B2 cells in the spleen was assessed every day until day 4 post-injection. The B2 cells were isolated at various time points from the spleen and analyzed by intracellular cytokine staining assay.

FIG. 15 is a flow cytometry bar graph showing the percentage of chemokine receptors for CXCR3⁺. The numbers in bar graph indicate the percent chemokine receptors expressing CD1⁹⁺CD5⁺CD23⁻ B-1a B cells. Data represent at least 3 independent experiments (*P<0.05; **P<0.01; ***P<0.001; ****P<0.0001).

FIG. 16 is a flow cytometry bar graph showing the percentage of chemokine receptors for CXCR4⁺. The numbers in bar graph indicate the percent chemokine receptors expressing CD19⁺CD5⁺CD23⁻B-1a B cells. Data represent at least 3 independent experiments (*P<0.05; **P<0.01; ***P<0.001; ****P<0.0001).

FIG. 17 is a flow cytometry bar graph showing the percentage of chemokine receptors for CXCR5⁺. The numbers indicate the percent chemokine receptors expressing CD19⁺CD⁵⁺CD23⁻B-1a B cells. Data represent at least 3 independent experiments (*P<0.05; **P<0.01; ***P<0.001; ****P<0.0001).

FIG. 18 is a set of fundus images of retinas showing improvement in clinical score following injection of IL-27. Experimental autoimmune uveitis (EAU) was induced by immunization of C57BL/6J mice with IRBP₆₅₁₋₆₇₀-peptide in Freund's adjuvant (CFA) (n =12). Mice were treated by intraperitoneal injection of IL-27 (100 ng/mouse) or PBS on day (−1) of immunization and every other day until day 12 post-immunization. Eyes were analyzed 14 days or 21 days post-immunization by fundoscopy, histology, optical coherence tomography (OCT), or electroretinography (ERG).

FIG. 19 is a graph showing the EAU scores of the retinas shown in FIG. 18. The EAU clinical scores and assessment of disease severity were based on changes at the optic nerve disc or retinal vessels as well as retinal and choroidal infiltrates.

FIG. 20 is a set of images of hematoxylin and eosin histological sections of the retinas of FIG. 18. Scale bar=200 μM; V=vitreous; GCL=ganglion cell layer; INL=inner nuclear layer; ONL=outer nuclear layer; RPE/CH=retinal pigmented epithelial and choroid.

FIG. 21 is a set of images showing the OCT analysis of the retinas of FIG. 18 showing the layered structure of the retina. The white arrows indicate inflammatory cells (white arrows) in the vitreous or optic nerve.

FIG. 22 is a graph showing the ERG analysis of a retina of FIG. 18 on day 20 after EAU induction. The averages of dark-adapted ERG a-wave amplitudes are plotted as a function of flash luminance, and the values are means ±SEM from 4 animals in each group.

FIG. 23 is a graph showing the ERG analysis of a retina of FIG. 18 on day 20 after EAU induction. The averages of dark-adapted ERG b-wave amplitudes are plotted as a function of flash luminance, and the values are means ±SEM from 4 animals in each group.

FIG. 24 is a graph showing the ERG analysis of a retina of FIG. 18 on day 20 after EAU induction. The averages of light-adapted ERG a-wave amplitudes are plotted as a function of flash luminance and values are means ±SEM from 4 animals in each group.

FIG. 25 is a graph showing the ERG analysis of a retina of FIG. 18 on day 20 after EAU induction. The averages of light-adapted ERG b-wave amplitudes are plotted as a function of flash luminance and values are means ±SEM from 4 animals in each group.

FIG. 26 is a graph showing the analysis of cytokine IL-27 in the serum of the mice of FIG. 18.

FIG. 27 is a graph showing the analysis of cytokine IL-17 in the serum of the mice of FIG. 18.

FIG. 28 is a graph showing the analysis of cytokine IL-10 in the serum of the mice of FIG. 18.

FIG. 29 is a graph showing the analysis of cytokine IL-35 in the serum of the mice of FIG. 18.

FIG. 30 is a flow cytometry plot showing the percentage of IL-27-expressing B cells. The numbers in the quadrants indicate the percent of CD19⁺ or CD19⁺CD5⁺CD1d^{hi} or CD19⁺CD5⁺CD1^{low} cells in the spleen of control (PBS-treated) or IL-27-treated EAU mice. The gating strategies are as indicated.

FIG. 31 is a bar graph showing the percentage of IL-27-expressing B cells of FIG. 30.

FIG. 32 is a flow cytometry plot showing the percentage of IL-27-expressing B cells in the spleen of control (PBS-treated) or IL-27-treated EAU mice. The gating strategies are as indicated. The numbers in the quadrants indicate the percent of CD19⁺ or CD19⁺CD5⁺CD1d^{hi} or CD19⁺CD5⁺CD1^{low} cells expressing p28, Ebi3, or p28 and Ebi3 (IL-27).

FIG. 33 is a bar graph showing the percentage of IL-27-expressing B-10 cells in the spleen of control (PBS-treated) or IL-27-treated EAU mice of FIG. 32.

FIG. 34 is a bar graph showing the percentage of IL-27-expressing B-1a cells in the spleen of control (PBS-treated) or IL-27-treated EAU mice of FIG. 32.

FIG. 35 is a set of fundus images of retinas from mice 17 days after adoptive transfer by fundoscopy. Purified peritoneal cavity B-la cells (5×10⁵ cells/mouse; >80% i27-Bregs) from wild type donor CD45.2⁺ EAU mice were transferred to naive syngeneic wild type or IL-27RαKO CD45.1⁺mice and 24 h after the adaptive transfer, EAU was induced in recipient mice by immunization with IRBP₆₅₁₋₆₇₀ (n=12). Clinical disease was monitored until 17 days after adoptive transfer by fundoscopy.

FIG. 36 is a graph showing the EAU scores of the retinas shown in FIG. 35.

FIG. 37 is set of flow cytometry plots from CD4⁺ T cells subjected to FACS and intracellular cytokine assays. The numbers in the quadrants indicate the percentage of CD4⁺ T cells expressing IL-17. Data represents at least 3 independent experiments (**P<0.01; ***P<0.001; ****P<0.0001).

FIG. 38A is a set of flow cytometry plots from CD4⁺ T cells subjected to FACS and intracellular cytokine assays. The numbers in the quadrants indicate the percentage of CD4⁺ T cells expressing IL-10.

FIG. 38B is a bar graph showing the percentage of CD4⁺ T cells expressing IFN-γ.

FIG. 38C is a bar graph showing the percentage of CD4³⁰ T cells expressing IL-17.

FIG. 38D is a bar graph showing the percentage of CD4⁺ T cells expressing IFN-γand IL-17.

FIG. 38E is a bar graph showing the percentage of CD4⁺ T cells expressing IL-10.

FIG. 39 is a set of flow cytometry plots from CD19⁺ T cells (eye) subjected to FACS. The numbers in the quadrants indicate the percentage of CD19⁺ CD5⁺ CD23⁻ B-1a cells expressing p28, p35, Ebi3, p28 and Ebi3 (IL-27). Data represents at least 3 independent experiments (**P<0.01; ***P<0.001;****P<0.0001).

FIG. 40 is a set of flow cytometry plots from CD19⁺ T cells (eye) subjected to FACS. The numbers in the quadrants indicate the percentage of CD19⁺ CD5⁻ CD23⁺ B2 expressing p28 and Ebi3 (IL-27) or p35 and Ebi3 (IL-35). Data represents at least 3 independent experiments (**P<0.01; ***P<0.001; ****P<0.0001).

FIG. 41 is a bar graph showing the percentage of B-la cells in the eye of FIG. 39 that express p28 and Ebi3.

FIG. 42 is a bar graph showing the percentage of B2 cells in the eye of FIG. 40 that express p28 and Ebi3.

FIG. 43 is a bar graph showing the percentage of B2 cells in the eye of FIG. 40 that express p35 and Ebi3.

FIG. 44 is a set of photomicrographs of hematoxylin and eosin stained sections of the brain (top row) and spinal cord (middle row) of mice on day 17 post-immunization (original magnification ×200). Arrows show inflammatory cells in the brain or spinal cord. The extent of EAE-induced demyelination was assessed by Luxol fast blue staining (bottom row; Luxol fast blue is a copper phthalocyanine dye that is soluble in alcohol and is attracted to bases found in the lipoproteins of myelin sheaths). Arrows denote areas of demyelination in the spinal cord. EAE was induced by immunization of C57BL/6J mice with MOG₃₅-55-peptide in CFA (n=12). Mice were treated by intraperitoneal injection of IL-27 (100 ng/mouse) or PBS on day 0 of immunization and every other day until day 12 post-immunization.

FIG. 45 is a graph showing the EAU scores of the spinal cord described in FIG. 44. The EAE clinical scores and disease assessment were ascertained by two masked investigators according to well established grading system.

FIG. 46 is a set of flow cytometry plots from inflammatory cells in the brain and spinal cord following intracellular cytokine analysis of untreated or IL-27-treated mice that were isolated day 17 post-immunization and then digested with collagenase. The numbers in quadrants indicate percentage of IL-17- or IFN-γ-expressing CD4 T cells in the spinal cord and brain.

FIG. 47 is a set of flow cytometry plots from inflammatory cells in the brain and spinal cord following intracellular cytokine analysis of untreated or IL-27-treated mice that were isolated day 17 post-immunization and then digested with collagenase. The numbers in quadrants indicate percentage of IL-10- expressing and CD4 T cells in the spinal cord and brain.

FIG. 48 is a bar graph showing the percentage of CD4 T cells of FIGS. 46 and 47 that express IFN-γ.

FIG. 49 is a bar graph showing the percentage of CD4 T cells of FIGS. 46 and 47 that express IL-17.

FIG. 50 is a bar graph showing the percentage of CD4 T cells of FIGS. 46 and 47 that express IL-17 and IFN-γ.

FIG. 51 is a bar graph showing the percentage of CD4 T cells of FIGS. 46 and 47 that express IL-10.

FIG. 52 is a set of flow cytometry plots showing the percentage of IL-27-expressing B cells from the spinal cord and brain of unimmunized, PBS-treated or IL-27-treated EAE mice analyzed for IL-27 (p28 and Ebi3) expression by intracellular cytokine staining assay. The numbers in the quadrants indicate the percentage of CD19⁺ CD5⁺CD1d^{hi} or CD19⁺CD5⁺CD1d^{low} B cells in the spinal cord or brain expressing p28, Ebi3 or p28 and Ebi3 (IL-27).

FIG. 53 is a bar graph showing the percentage of CD19 T cells of FIG. 52 that express CD19⁺CD5⁺CD1d^{low}.

FIG. 54 is a set of flow cytometry plots showing the percentage of IL-27-expressing B cells from the spleen of unimmunized, PBS-treated or IL-27-treated EAE mice analyzed for IL-27 (p28 and Ebi3) expression by intracellular cytokine staining assay. The numbers in the quadrants indicate the percentage of total CD19⁺CD5⁺CD1d^{hi} or CD19⁺CD5⁺CD1d^{low} B cells in the spleen expressing p28, Ebi3 or p28 and Ebi3 (IL-27).

FIG. 55 is a bar graph showing the percentage of CD19 T cells of FIG. 54 that express p28 and Ebi3.

FIG. 56 is a set of flow cytometry plots showing analysis of spleen cells of PBS-treated or IL-27-treated EAE mice for IL-27 expansion. The numbers in the quadrants indicate the percentage of CD19⁺CD5⁺CD1d^{low} B-1a cells.

FIG. 57 is a bar graph showing the percentage of CD19 T cells of FIG. 56 that express CD19⁺CD5⁺CD1d^{hi}.

FIG. 58 is a bar graph showing the percentage of CD19 T cells of FIG. 56 that express CD19⁺CD5⁺CD1d^{hi}.

FIG. 59 is a graph showing the EAU scores of spleen cells from MOG₃₅₋₅₅ immunized (PBS-treated EAE or IL-27-treated) CD45.2⁺ mice that were re-stimulated ex-vivo and transferred (1×10⁷ cells/mouse) to naïve CD45.1⁺WT mice. The EAE clinical scores and disease assessment were ascertained by two masked investigators.

FIG. 60 is a set of flow cytometry plots showing the percentage of CD4⁺ T cells. Spinal cord, brain, lymph nodes (LN) or spleen of PBS-treated or IL-27-treated mice were isolated on day 20 post-adoptive transferred, digested with collagenase and CD4⁺ T cells and IL-27-producing B-1a and analyzed by intracellular cytokine staining assay. The numbers in the quadrants indicate the percentage of CD4⁺ T cells expressing IL-17 or IFN-γ. Data represents >3 independent experiments (**P<0.01; ***P<0.001; ****P<0.0001).

FIG. 61 is a bar graph showing the percentage of the cells of FIG. 60 that express IL-17.

FIG. 62 is a bar graph showing the percentage of the cells of FIG. 60 that express IL-17 and IFN-γ.

FIG. 63 is a set of flow cytometry plots showing the percentage of IL-27-producing B-1a cells. Spinal cord, brain, lymph nodes (LN) or spleen of PBS-treated or IL-27-treated mice were isolated on day 20 post-adoptive transferred, digested with collagenase and CD4⁺ T cells and IL-27-producing B-1a, and analyzed by intracellular cytokine staining assay. The numbers in the quadrants indicate the percentage of CD19⁺CD5⁺CD11b⁺B-1a cells expressing p28, Ebi3 or p28 and Ebi3 (IL-27). Data represents >3 independent experiments (**P<0.01; ***P<0.001; ****P<0.0001).

FIG. 64 is a bar graph showing the percentage of the B-1a cells of FIG. 63 from the spinal cord that express p28 and Ebi3 (IL-27).

FIG. 65 is a bar graph showing the percentage of the B-1a cells of FIG. 63 from the brain that express p28 and Ebi3 (IL-27).

FIG. 66 is a bar graph showing the percentage of the B-1a cells of FIG. 63 from the spleen that express p28 and Ebi3 (IL-27).

FIG. 67 is a set of flow cytometry plots (top) and a graph showing EAE clinical scores (bottom) from purified peritoneal cavity B-1a cells (5×10⁵ cells/mouse; >80% i27-Bregs) from WT donor CD45.2⁺ mice that were transferred to naive syngeneic wild type mice and 24 h after the adaptive transfer, EAE was induced in recipient mice by immunization with MOG35-55 (n=12). The EAE clinical scores and disease assessment as performed by two masked investigators.

FIG. 68 is a set of flow cytometry plots showing the percentage of CD4⁺ T cells expressing IL-10, IL-17, or IFN-γ. Spinal cords and brains of PBS-treated or B-1a-treated mice were isolated on day 15 post-immunization, digested with collagenase and analyzed by an intracellular cytokine staining assay. Data represents >3 independent experiments (**P<0.01; ***P<0.001; ****P<0.0001).

FIG. 69 is a bar graph showing the percentage of spinal cord and brain cells of FIG. 68 that express IFN-γ.

FIG. 70 is a bar graph showing the percentage of spinal cord and brain cells of FIG. 68 that express IL-17.

FIG. 71 is a bar graph showing the percentage of spinal cord and brain cells of FIG. 68 that express IL-10.

FIG. 72 is a set of flow cytometry plots showing the percentage of CD19⁺CD5⁺CD23⁻B-1a or CD19+CD5⁻CD23⁺B2 cells expressing p28, Ebi3 or p28 and Ebi3 (IL-27) in spinal cords. Spinal cords of PBS-treated or B-la-treated mice were isolated on day 15 post-immunization, digested with collagenase and analyzed by an intracellular cytokine staining assay. Data represents>3 independent experiments (**P<0.01; ***P<0.001; ****P<0.0001).

FIG. 73 is a bar graph showing the percentage of spinal cord cells of FIG. 72 that express p28 and Ebi3 (IL-27).

FIG. 74 is a set of flow cytometry plots showing the percentage of CD19⁺CD5⁺CD23⁻B-1a or CD19⁺CD5⁻CD23⁺B2 cells expressing p28, Ebi3 or p28 and Ebi3 (IL-27) in brains. Brains of PBS-treated or B-1a-treated mice were isolated on day 15 post-immunization, digested with collagenase and analyzed by an intracellular cytokine staining assay. Data represents >3 independent experiments (**P<0.01; ***P<0.001; ****P<0.0001).

FIG. 75 is a bar graph showing the percentage of brain cells of FIG. 74 that express p28 and Ebi3 (IL-27).

FIG. 76 is a set of flow cytometry plots showing the percentage of CD19⁺CD5⁺CD23⁻B-1a or CD19⁺CD5⁻CD23⁺cells expressing p28, Ebi3 or p28 and Ebi3 (IL-27) in peritoneal cavities. Fluids from peritoneal cavities of PBS-treated or B-1a-treated mice were isolated on day 15 post-immunization, digested with collagenase and analyzed by an intracellular cytokine staining assay. Data represents >3 independent experiments (**P<0.01; ***P<0.001; ****P<0.0001).

FIG. 77 is a bar graph showing the percentage of cells of FIG. 74 from the peritoneal cavities that express p28 and Ebi3 (IL-27).

FIG. 78 is a depiction that illustrates macrophages from EAU mice being cultured in a trans-well system containing B-1a cells from wild type EAU mice at the bottom wells. The effects of the macrophages on proliferation of B-1a cells was assessed by [³H]-thymidine incorporation assays.

FIG. 79 is a set of flow cytometry plots showing the percentage of B-1a cells of FIG. 78 that express p28, Ebi3 or p28 and Ebi3 (IL-27).

FIG. 80 is a bar graph showing the CPM mean values of the B-1a cells and macrophages of FIG. 78. The proliferative responses were analyzed in 5 replicate cultures. Data represents at least 3 independent experiments (**P<0.01; ***P<0.001; ****P<0.0001).

FIG. 81 is a bar graph showing the percentage of B-1a cells and macrophages of FIG. 78 that express p28 and Ebi3 (IL-27).

FIG. 82 is a graph showing the ELISA analysis of the secretion of IL-27 of primary mouse peritoneum macrophages that were activated with LPS in the presence or absence of lentivirus guide RNA that targets p28 (sgp28-1 or spg28-2).

FIG. 83 is a graph showing the ELISA analysis of the secretion of IL-27 of primary mouse peritoneum B-1a cells that were activated with LPS in the presence or absence of lentivirus guide RNA that targets p28 (sgp28-1 or spg28-2).

FIG. 84 is a depiction that illustrates pathogenic (uveitogenic) T cells from EAU mice being cultured in a trans-well system containing B-1a cells infected with lentivirus guide RNA that targets suppression of IL-27 (sgp28/Ebi3). The effects of the B-1a cells on the proliferation of the uveitogenic T cells was assessed by [³H]-thymidine incorporation assays.

FIG. 85 is a bar graph showing the CPM mean values of the cells of FIG. 84.

FIG. 86 is a set of flow cytometry plots showing the percentage of uveitogenic CD4⁺ T cells expressing IL-10, IL-17 and/or IFN-γas determined by an intracellular cytokine staining assay.

FIG. 87 is a bar graph showing the percentage of cells of FIG. 86 that express IFN-γ.

FIG. 88 is a bar graph showing the percentage of cells of FIG. 86 that express IL-17.

FIG. 89 is a bar graph showing the percentage of cells of FIG. 86 that express IFN-γand IL-17.

FIG. 90 is a bar graph showing the percentage of cells of FIG. 86 that express IL-10.

FIG. 91 is a depiction that illustrates pathogenic (uveitogenic) T cells from EAU mice being cultured in a trans-well system containing B-1a cells from wild type EAU mice or B-1a cells infected with lentivirus guide RNA that targets suppression of IL-27 (sgp28/Ebi3). The effects of the B-1a cells on the proliferation of the uveitogenic T cells was assessed by [³H]-thymidine incorporation assays.

FIG. 92 is a set of flow cytometry plots showing the percentage of uveitogenic CD4⁺ T cells expressing LAG-3 as determined by an intracellular cytokine staining assay.

FIG. 93 is a bar graph showing the percentage of the cells of FIG. 92 that express LAG-3.

FIG. 94 is a depiction that illustrates pathogenic (uveitogenic) T cells from EAU mice being cultured in a trans-well system containing B-1a cells from wild type EAU mice or B-1a cells infected with lentivirus guide RNA that targets suppression of IL-27 (sgp28/Ebi3). The effects of the B-1a cells on the proliferation of the uveitogenic T cells was assessed by [³H]-thymidine incorporation assays.

FIG. 95 is a set of flow cytometry plots showing the percentage of CD4⁺CD25⁺Foxp3⁺ and CD4⁺CD25⁺ Foxp3⁻ expressing p35, Ebi3, or IL-35 (p35/Ebi3).

FIG. 96 is a bar graph showing the percentage of the cells of FIG. 95 that express p35 and Ebi3.

FIG. 97 is a set of flow cytometry plots showing the percentage of CD4⁺CD25⁺Foxp3⁺ and CD4⁺CD25⁺Foxp3⁻ expressing p35, Ebi3, or IL-35 (p35/Ebi3).

FIG. 98 is a bar graph showing the percentage of the cells of FIG. 97 that are CD4⁺CD25⁺Foxp3⁺.

FIG. 99 is a bar graph showing the percentage of the cells of FIG. 97 that are CD4⁺CD25⁺Foxp3⁻.

FIG. 100 is a flow cytometry plot and a set of graphs that show the results of sorted CD19⁺CD5⁺CD23⁻B-1a cells from the peritoneal cavity of C57BL/6J mice that were activated in vitro for 48 h by stimulation with LPS analyzed by ELISA (left flow cytometry plot). The supernatants from the cultures in the peritoneal cavity were analyzed by qPCR (right).

FIG. 101 is a bar graph showing the results of qPCR analysis of purified B-la cells from the peritoneal cavities of C57BL/6J mice injected (i.v) 48 hours prior with LPS.

FIG. 102 is a flow cytometry plot showing B-1a or plasma cells (B2) from C57BL/6J mouse peritoneal cavity or spleen, respectively, after sorting using magnetic beads and then activated with anti-CD40/anti-IgM (BCR).

FIG. 103 is a graph of the qPCR analysis of RNA from the cells of FIG. 102 that was quantified for expression of Pdl mRNA transcript.

FIG. 104 is a graph of the qPCR analysis of RNA from the cells of FIG. 102 that was quantified for expression of Lag3 mRNA transcript.

FIG. 105 is a set of graphs showing RNA isolated at various time points analyzed by qRT-PCRC D19⁺ B cells from C57BL/6J mouse spleen that were activated with anti-CD40/anti-IgM in the presence or absence of IL-27.

FIG. 106 is the Volcano plot analysis of the genes differentially induced by IL-27 24 h after they were detected using a NanoString transcription factor panel.

FIG. 107 is an image of a Western blot. CD19⁺ B cells were isolated from the spleen of C57BL/6J mouse 24 h after immunization with LPS in the presence or absence of IL-27 (in-vivo) or from mouse CD19⁺ B cells after activated with LPS in the absence or presence of IL-27 (in vitro). Nuclear extracts were prepared from the cells and analyzed by the electrophoretic mobility shift assay (EMSA) to detect IL-27-induced AICE complexes. Transcription factors recruited to the CTLA4-AICE or p28-AICE locus were identified by super-shift assay. Whole cell extracts prepared from CD19⁺ B cells of the C57BL/6J mouse immunized with LPS in the presence or absence of IL-27 were analyzed by Western blotting.

FIG. 108 is a bar graph showing the relative gene expression of B-1a cells from the peritoneal cavity.

FIG. 109 is a set of flow cytometry plots of the FACS analysis of CD19⁺ B cells showing the percentage of B-1a or plasmablasts expressing p28, Ebi3, or p28 and Ebi3 (IL-27). The CD19⁺ B cells from the spleen of C57BL/6J (wild type) or mice with targeted deletion of irf8 in B cells (CD19-IRF8KO) following activation for 3 days with anti-CD40/anti-IgM. The gating strategy is as indicated. Data represents >3 independent experiments (**P<0.01; ***P <0.001; ****P <0.0001).

FIG. 110 is a bar graph showing the amount of CD19⁺CD27⁺CD383⁺ cells of FIG. 109.

FIG. 111 is a bar graph showing the amount of CD19⁺CD5⁺CD11b⁺ cells of FIG. 109.

FIG. 112 is a bar graph showing a chromatin immunoprecipitation (CHIP) analysis that was performed with B-1a cells stimulated with LPS or LPS+IL-27 for 24 h and STAT1 binding to the p28 or ebi3 promoter region was analyzed. Cell lysates were immunoprecipitated with anti-STAT1 antibody or control IgG. Immunoprecipitated and input DNA were analyzed by qPCR using primers corresponding to p28 or ebi3 promoter sites.

FIG. 113 is a bar graph showing a CHIP analysis that was performed with B-1a cells stimulated with LPS or LPS+IL-27 for 24 h and STAT3 binding to the p28 or ebi3 promoter region was analyzed. Cell lysates were immunoprecipitated with anti-STAT3 antibody or control IgG. Immunoprecipitated and input DNA were analyzed by qPCR using primers corresponding to p28 or ebi3 promoter sites.

FIG. 114 is a set of flow cytometry plots of the FACS analysis of healthy human PBMC that were cultured for 3 days with TLR9 agonist CpG and BCR (anti-CD40 or anti-IgM). Gating on human B-1 cells (CD19⁺CD20⁺CD27⁺CD43⁺) revealed that as high as 19.9% of BCR-activated B-cells in human PBMC produced IL-27.

FIG. 115 is a bar graph showing the amount of CD19⁺CD2O^{+CD}27⁺CD43⁺p28⁺Ebi3⁺cells of FIG. 114.

FIG. 116 is a set of flow cytometry plots of the FACS analysis of healthy human PBMC that were cultured for 3 days with TLR9 agonist CpG and BCR (anti-CD40 or anti-IgM). Gating on CD19⁺CD20⁺CD27⁺CD43⁺CD1 lb⁺B-1 cells revealed as many as 35% of BCR-activated human B-1 cells.

FIG. 117 is a bar graph showing the amount of CD19⁺CD2O⁺CD27⁺CD43⁺CD11b⁺p28+Ebi3⁺cells of FIG. 116.

FIG. 118 is a bar graph showing the amount of CD19⁺CD20+CD27+CD43+CD11b⁻p28+Ebi3+cells of FIG. 116.

FIG. 119 is a set of flow cytometry plots of the FACS analysis of human umbilical cord blood from healthy human donors. As many as 18.1% of resting B-1a cells constitutively produced IL-27 and stimulation of BCR-activated cord blood B-cells with IL-27 increased the percentage of cord blood i27-Bregs to 73.9%.

FIG. 120 is a bar graph showing the amount of cells of FIG. 119 (top panel).

FIG. 121 is a bar graph showing the amount of cells of FIG. 119 (bottom panel).

FIG. 122 is a set of graphs showing the relative abundance of i27-Bregs as compared to other Breg subtypes (IL-10-producing Bregs and i35-Bregs). Activated human cord blood cells were propagated for 6 days. The majority of the Breg cells were i27-Bregs (largest slice of each pie graph, ranging from 61.2 +/−5.3 to 87.1 +/−3.1%) and much lower levels of IL-10-producing Bregs (ranging from 2.6 +/ −0.3 to 6.7 +/−1.1%) and i35-Bregs (ranging from 10.2 +/−2.7 to 32 +/−6.8 5) were detected.

FIG. 123 is a set of graphs showing the relative abundance of B-2 cells. These plots revealed that most i27-Bregs were either in the naïve or memory B-cell pool.

FIG. 124 is a graph showing that similar to the mouse species, the human i27-Breg cells constitutively express inhibitory receptors PD-1 and LAG3.

FIG. 125 is a bar graph showing the amount of PD-1⁺ cells of FIG. 124.

FIG. 126 is a bar graph showing the amount of LAG3+ cells of FIG. 124.

FIG. 127 is a set of flow cytometry plots of the FACS analysis of human i27-Breg cells. The i27-Breg cells suppressed proliferative responses of TNF-α-, IL-17-, and IFN-γ-producing pro-inflammatory CD4⁺ T-cells.

FIG. 128 is a bar graph showing the CPM mean values of the cells of FIG. 127.

FIG. 129 is a bar graph showing the amount of TNF-α+CD4⁺T cells of FIG. 127.

FIG. 130 is a bar graph showing the amount of IFN-γ+CD4⁺T cells of FIG. 127.

FIG. 131 is a bar graph showing the amount of IL-17A+CD4⁺T cells of FIG. 127.

FIG. 132 shows the results from a Proximity Ligation Assay (PLA) which shows the physical interaction between p28 and Ebi3.

FIG. 133 is a gel showing that B-cells produce the heterodimeric (p28/Ebi3) IL-27 cytokine. C57BL/6J mice were injected (i.v) with LPS or LPS+IL-27 and after 24 h lysates or supernatant from cultured B-1a cells were subjected to reciprocal immunoprecipitation/Western blot analysis. The antibodies used for IP or Western blotting are indicated.

FIG. 134 is a graph that shows the results of NanoString RNA analysis showing that IL-27 altered the pattern of chemokine receptor expression in activated B cells.

FIG. 135A shows a flow cytometry plot depicting the differential secretion of natural IgM antibodies by unchallenged B-1a and i27-Breg cells in the peritoneal cavity.

FIG. 135B shows a set of graphs depicting the differential secretion of natural IgM antibodies by unchallenged B-1a and i27-Breg cells in the peritoneal cavity.

FIG. 136 shows the Principal Component Analysis (PCA) plot depicting segregation of the cells into 4 distinct populations.

FIG. 137 shows Gene ontology (GO) analysis showing functional pathway enrichment for i27-Breg cells.

FIG. 138 is a heatmap of the i27-Breg cells gene signature in comparison to signature program of the unchallenged B-1a cell.

FIG. 139 is set of heatmaps showing genes that encode transcription factors, signaling proteins, cytokines and chemokines or cell surface proteins which are differentially expressed genes in i27-Bregs and B-1a cells.

FIG. 140 is a set of graphs from qPCR expression of genes encoding inhibitory receptors.

FIG. 141A is a set of representative flow cytometry plots showing significant expansion of IL-27 secreting CD19⁺CD20⁺CD27⁺CD43⁺B-1 cells in response to anti-CD40 or BCR.

FIG. 141B is a scatter plot showing significant expansion of IL-27 secreting CD19⁺CD20⁺CD27⁺CD43⁺B-1 cells in response to anti-CD40 or BCR.

FIG. 142A is a set of representative flow cytometry plots showing significant expansion of IL-27 secreting CD27⁺CD43⁺CD11⁺or CD27⁺CD43⁺CD11⁻B-1 cells in response to anti-CD40 or BCR.

FIG. 142B is a set of scatter plots showing significant expansion of IL-27 secreting CD27⁺CD43⁺CD11⁺or CD27⁺CD43⁺CD11⁻B-1 cells in response to anti-CD40 or BCR.

FIG. 143A is a set of representative flow cytometry plots of human cord blood CD19⁺B cells (top) or sorted B-1a cells in the blood (bottom) activated with BCR or BCR plus IL-27 showing significant expansion IL-27-producing CD27⁺CD43⁺B-1a cells.

FIG. 143B is a set of scatter plots of human cord blood CD19⁺B cells (top) or sorted B-1a cells in the blood (bottom) activated with BCR or BCR plus IL-27 showing significant expansion IL-27-producing CD27⁺CD43⁺B-1a cells.

FIG. 144 shows representative t-SNE clustering plots and flow cytometry pie charts showing the distribution and relative abundance of IL-27 (i27-Breg), IL-35 (i35-Breg), and IL-10-secreting Bregs in the B-1 compartment of activated human umbilical cord blood.

FIG. 145 are graphs and pie charts showing the amount of various Breg subsets (e.g., i27-Bregs, i35-Bregs, and B10 cells) in cultures after human CD19⁺ B cells in human blood were activated for 6 days and analyzed by intracellular cytokine assay.

FIG. 146 shows the results of RNA-Seq analysis using RNA from the conventional CD19⁺B-2, i27-Breg, i35-Breg, or B10 cells.

FIG. 147 is a graph that depicts a heat map analysis showing genes that are differentially expressed between i27-Breg and i35-Breg cells.

FIG. 148 is a graph that depicts a heat map analysis showing genes that are differently expressed between conventional CD19⁺B-2 and i27-Breg cells.

FIG. 149A is set of representative flow cytometry plots showing the percentage of IL-27 secreting CD11 B-1a cells following co-culture of activated IL-27-producing B-la and plasmacytoid dendritic cells (1:1).

FIG. 149B is representative bar graph showing the percentage of IL-27 secreting CD11b^{+B-}1a cells following co-culture of activated IL-27-producing B-1a and plasmacytoid dendritic cells (1:1).

FIG. 150 is a scatter plot showing significant suppression of EAE after purified IL-27-secreting peritoneal B-1a cells (>80% i27-Bregs) from WT donor CD45.2⁺ mice were transferred (5×10⁵ cells/mouse) to naïve syngeneic CD45.1⁺mice and 24 h later EAE was induced in the recipient mice by immunization with MOG35-55(n=12).

FIG. 151A is a set of representative flow cytometry plots showing reduced EAE symptoms in mice treated with i27-Bregs as shown by percentage of CD4⁺ T cells expressing IL-10, IL-17 or IFN-γ.

FIG. 151B is a set of scatter plots showing reduced EAE symptoms in mice treated with i27-Bregs as shown by percentage of CD4⁺T cells expressing IL-10, IL-17 or

FIG. 152A is a set of representative flow cytometry plots showing CD19⁺CD5⁺CD23⁻B-1a or CD19⁺CD5⁻CD23+B2 cells secreting IL-27 in the spinal cord.

FIG. 152B is a set of scatter plots showing CD19⁺CD5⁺CD23⁻B-1a or CD19⁺CD5⁻CD23⁺B2 cells secreting IL-27 in the spinal cord.

FIG. 153A is a set of representative flow cytometry plots showing CD19⁺CD5⁺CD23⁻B-1a or CD19⁺CD5⁻CD23⁺B2 cells secreting IL-27 in the brain.

FIG. 153B is a set of scatter plots showing CD19⁺CD5⁺CD23⁻B-1a or CD19⁺CD5⁻CD23⁺B2 cells secreting IL-27 in the brain.

FIG. 154A is a set of representative flow cytometry plots showing CD19⁺CD5⁺CD23⁻B-1a or CD19⁺CD5⁻CD23⁺B2 cells secreting IL-27 in the peritoneal cavity.

FIG. 154B is a set of scatter plots showing CD19⁺CD5⁺CD23⁻B-1a or CD19⁺CD5⁻CD23⁺B2 cells secreting IL-27 in the peritoneal cavity.

### DETAILED DESCRIPTION OF THE INVENTION

Regulatory B-cells (Bregs) suppress autoimmune diseases through production of IL-10 or IL-35 alone or in combination with inhibitory cell-surface receptors. However, Bregs described thus far (e.g., U.S. Pat. No. 9,629,897) are antigen-specific and derive from B2-lymphocyte lineage. The invention provides an isolated population of human cells comprising a non-naturally occurring, concentrated population of regulatory B-cells of B-1a lineage that produce and secrete interleukin-27 (i27-Bregs).

Interleukin-27 (IL-27) is a member of the IL-12 cytokine family. IL-27 is a heterodimeric cytokine that is composed of two distinct protein subunits encoded by ebi3 (Epstein-Barr virus-induced gene 3) and IL-27p28. IL-27 is expressed by cells and interacts with IL-27 receptor (IL-27R). IL-27R consists of two proteins, IL-27a (IL-27 alpha) and gp130. IL-27 induces differentiation of the diverse populations of T cells in the immune system. Natural activation of B-1a regulatory cells upon inflammatory stimuli triggers IL-27 production and the coincident exodus of i27-Bregs to the spleen where they reprogram conventional lymphocytes to acquire immune-regulatory functions.

The population of cells of the invention can comprise about 25% or more B-la regulatory cells (e.g., about 30% or more, about 35% or more, about 40% or more, about 45% or more, about 55% or more, about 60% or more, about 65% or more, about 70% or more, about 75% or more, about 80% or more, about 81% or more, about 82% or more, about 83% or more, about 84% or more, about 85% or more, about 86% or more, about 87% or more, about 88% or more, about 89% or more, or about 90% or more B-1a regulatory cells). Populations of B-1a cells at such relatively high proportions compared to other cell types within the population of cells do not exist in the human body or in nature. B-1a cells within the human body are detected at low numbers in peripheral lymphoid tissues (<2%). Within this minority population of less than 2%, i27-Bregs comprise less than 2%—only up to about 4/10,000 of a naturally occurring human cell population (i.e., 0.02×0.02 =0.0004).

The population of cells of the invention expresses the cell surface inhibitory receptors lymphocyte-activation gene 3 (LAG-3), programmed cell death protein 1 (PD-1), and C-X-C chemokine receptor type 4 (CXCR4). The population of cells can have the receptors on their surfaces or be capable of having the receptors on their surfaces.

LAG-3 (or cluster of differentiation 223 (CD223)) is a protein encoded by the LAG3 gene in humans. LAG3 is an immune checkpoint receptor.

PD-1 (or cluster of differentiation 279 (CD279)) is a protein encoded by the PDCD1 gene in humans. PD-1 is also an immune checkpoint receptor. PD-1 promotes apoptosis of antigen-specific T-cells in lymph nodes and reduces apoptosis in regulatory T cells (anti-inflammatory, suppressive T cells).

CXCR4 (or fusin or cluster of differentiation 184 (CD184)) is a protein encoded by the CXCR4 gene in humans. CXCR4 is an alpha-chemokine receptor specific for stromal-derived-factor-1 (SDF-1 or CXCL12), a molecule with chemotactic activity for lymphocytes.

The population of cells optionally also expresses the cell surface inhibitory receptor glucocorticoid-induced TNFR-related protein (GITR or tumor necrosis factor receptor superfamily member 18 (TNFRSF18) or activation-inducible TNFR family receptor (AITR)). GITR is a protein encoded by the TNFRSF18 gene in humans. GITR has been shown to have increased expression upon T-cell activation.

The population of cells of the invention optionally also expresses the cell surface inhibitory receptor OX40 (or tumor necrosis factor receptor superfamily member 4 (TNFRSF4) or cluster of differentiation 134 (CD134)). OX40 is a protein encoded by the TNFRSF4 gene in humans. OX40 is not constitutively expressed on resting naïve T cells.

The population of cells of the invention optionally also expresses the cell surface inhibitory receptor cytotoxic T-lymphocyte-associated protein 4 (CTLA4 or cluster of differentiation 152 (CD152)). CTLA4 is a protein encoded by the CTLA4 gene in humans. CTLA4 is an immune checkpoint and downregulates immune responses. CTLA4 is constitutively expressed in regulatory T cells but only upregulated in conventional T cells after activation.

The population of cells of the invention can be from a mammal. The term “mammal” includes, but is not limited to, the order Rodentia, such as mice, and the order Logomorpha, such as rabbits, the order Carnivora, including Felines (cats) and Canines (dogs), the order Artiodactyla, including Bovines (cows) and Swines (pigs), the order Perssodactyla, including Equines (horses), Primates, Ceboids, or Simioids (monkeys), and the order Anthropoids (humans and apes). More preferably, the population of cell are from a human.

The invention provides methods of preparing the population of cells (e.g., human cells) comprising (a) isolating cluster of differentiation 5 positive (CDS⁺) expressing cells from a mammal tissue or fluid sample to provide isolated CD5+ expressing cells, (b) culturing the isolated CDS+expressing cells in a cell culture media to provide cultured cells, (c) activating the cultured cells with a BCR (B cell receptor) or a TLR (Toll-like receptor) agonist to provide activated cells; and (d) exposing the activated cells to IL-27. In this regard, the isolating of the CD5+ (CD5 is expressed on the surface of T cells and B-1a cells) expressing cells can be carried out by any suitable method, for example by using fluorescence-activated cell sorting (FACS), microfluidic cell sorting, or magnetic cell sorting.

The mammal tissue or fluid sample can be from any suitable source, such as mammal peripheral lymphoid tissue, mammal cord blood, mammal peritoneal fluid, mammal bone marrow, induced pluripotent cells (iPSC), or any other sample containing B-1a cells. In at least some embodiments, the use of peritoneal fluid or cord blood as the sample may be desirable because these sources typically have a higher percentage of B-1a cells than other samples (e.g., peripheral lymphoid tissue). In some embodiments, the preferred source of the tissue or fluid may be from the donor subject that will be treated with the population of cells of the invention.

Any suitable cell culture media that can support the growth of B-1a cells can be used. For example, Roswell Park Memorial Institute medium (RPMI 1640) culture medium can be used.

The cultured cells are exposed to a BCR agonist or a TLR agonist. Any suitable BCR agonist or a TLR agonist that can activate the cells can be used. Examples of BCR agonists include anti-CD40 and anti-IgM antibodies. Examples of TLR agonists include TLR9 and TLR4 agonists. As is the case for all lymphocytes, the B-1a cells have to be activated to elicit biological activity and thus the CD5+B-1a cells activated with a BCR agonist or TLR agonist. CD40 is a costimulatory protein found on antigen presenting cells and is required for B cell activation following interaction of the B cell receptor with antibody to IgM. However, maximum secretion of IL-27 by the activated B-1a cell requires IL-27 signals provided by binding of IL-27 to its cognate receptor on the B-1a cell and further upregulation of the IL-27 receptor.

As used herein, the terms “Toll-like receptor” and “TLR” refer to any member of a family of highly-conserved mammalian proteins which recognize pathogen-associated molecular patterns and act as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 4” and “TLR4” refer to nucleic acids or polypeptides sharing at least 70%, 80%, 90%, 95%, 96%, 97%, 98%, 99%, or more sequence identity to a publicly-available TLR4 sequence. A suitable TLR 4 agonist is LPS.

The terms “Toll-like receptor 9” and “TLR9” refer to nucleic acids or polypeptides sharing at least 70%, 80%, 90%, 95%, 96%, 97%, 98%, 99%, or more sequence identity to a publicly-available TLR9 sequence. Suitable TLR9 agonists are oligonucleotides containing CpG motifs (CpG ODNs).

The activated cells are exposed to IL-27. The exposure to IL-27 facilitates expansion of the i27-Bregs and creates an efficient ongoing increase in the proportion and amount of i27-Bregs.

The inventive methods are useful for the treatment of a disease in a mammal. The treatment may result in desirable suppression of the immune system.

The inventive methods are useful for the treatment, suppression, or prevention of GVHD. Patients can receive a solid organ or allogeneic bone marrow or hematopoietic stem cell transplant. In order to prevent or reduce the severity of GVHD, the population of mammal cells of the invention are administered to a mammal before the mammal receives an allogeneic transplant. Alternatively, GVHD can be prevented or suppressed by mixing the i27-Breg population of cells of the invention with a transplant material to form a transplant mixture, and then administering the transplant mixture to the mammal. In this regard, the transplant material can include allogeneic lymphocytes. In an embodiment, the transplanted cells are cells (e.g., heart cells, pancreatic cells, retinal cells) derived from iPS cells.

The population of mammal cells of the invention can be mixed with the transplant material ex vivo. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context.

The population of mammal cells can be administered in the form of a pharmaceutically acceptable (e.g., physiologically acceptable) composition. The composition may comprise a carrier, preferably a pharmaceutically (e.g., physiologically acceptable) carrier, and the population of mammal cells. Any suitable carrier can be used within the context of the invention, and many such carriers are known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The population of mammal cells can be administered to a mammal (as earlier defined herein). Preferably the mammal is a mouse or a human.

The invention provides a method of suppressing the immune system in a mammal, which method comprises administering the population of mammal cells of the invention to a mammal in need thereof, thereby suppressing the immune system in the mammal. Thus, the invention provides for a method of suppressing autoimmunity in a mammal comprising administering an isolated IL-27-producing B-1a cell population to a mammal whereupon the in vivo IL-27 production in the mammal is increased to artificially high levels and autoimmunity is thereby suppressed in the mammal. IL-27 is rapidly cleared in vivo, however, the administration of the isolated IL-27-producing B-1a cell population allows for proliferation of i27-Bregs and sustained IL-27 secretion in vivo. This provides distinct advantages over therapies that may rely upon direct administration of IL-27.

IL-27 and IL-35 are the two immune-suppressive members of the IL-12 family of cytokines. Although IL-35 or IL-27 show substantial promise in suppressing autoimmune diseases, a major disadvantage of using cytokines as biologics, especially heterodimeric cytokines, is their relatively short half-life, transient biological activities and unpredictable pharmacokinetic characteristics. Another important impediment relates to the issue of dosing. Because association of the IL-35 or IL-27 subunit proteins is not strong (non-covalent), IL-35 and IL-27 subunit proteins readily dissociate making it difficult to ascertain the effective dose of bioactive p35:Ebi3 or p28:Ebi3 heterodimer administered or required to ameliorate disease. Therapeutic use of i27-Bregs provides several therapeutic advantages over the use of biologics such as IL-10, IL-27 or IL-35, which are the most effective cytokines produced by Breg or Treg cells: (i) the ex-vivo generated i27-Bregs proliferated in-vivo and thereby sustained production of IL-27 in recipient host tissues; (ii) the ex-vivo generated i27-Bregs proliferated in-vivo and reprogram recipient lymphocytes into IL-10-, IL-27, IL-35-producing Bregs and Tregs, and can thereby sustained production of these immune suppressive cytokines in recipient host tissues; (iii) disease suppression by innate i27-Bregs does not require prior activation by autoantigen that elicits disease, providing potential therapeutic advantage over disease-specific Breg/Treg therapies used for autoimmune diseases.

The term “autoimmunity,” as used herein, refers to the failure of an organism (e.g., a mammal, such as a human or mouse) to recognize its own constituent parts as self, which results in an immune response against the organism's own cells and tissues. In other words, autoimmunity is an adaptive immune response directed against “self” antigens and is marked by the production of proinflammatory cytokines that mediate pathology by damaging host tissues or by production of “autoantibodies” that can cause complement mediated diseases.

“Autoimmune disease” refers to any one of a group of diseases or disorders in which tissue injury is associated with a humoral and/or cell-mediated immune response to body constituents or, in a broader sense, an immune response to self. The pathological immune response may be systemic or organ specific. For example, the immune response directed against self may affect joints, skin, the brain, the myelin sheath that protects neurons, the kidneys, the liver, the pancreas, the thyroid, the adrenals, the eyes (e.g., uveitis), and ovaries. Immune complex formation plays a role in the etiology and progression of autoimmune disease. Increased immune complex formation correlates with the presence of antibodies directed to self (autoantibodies). The presence of autoantibodies can contribute to tissue inflammation either as part of an immune complex or unbound to antigen (free antibody). In some autoimmune diseases, the presence of free autoantibody contributes significantly to disease pathology. Another aspect of the etiology and progression of autoimmune disease is the role of proinflammatory cytokines. Under normal circumstances, proinflammatory cytokines such as tumor necrosis factor-a (TNF-a) and interleukin-1 (IL-1) play a protective role in the response to infection and cellular stress. However, the pathological consequences which result from chronic and/or excessive production of TNF-a and IL-1 are believed to underlie the progression of many autoimmune diseases such as rheumatoid arthritis, Crohn's disease, inflammatory bowel disease, uveitis, and psoriasis. Other proinflammatory cytokines involved in autoimmune disease include interleukin-6, interleukin-8, and granulocyte-macrophage colony stimulating factor (see, e.g., U.S. Pat. No. 8,080,555).

The inventive cell population and methods can be used to suppress autoimmunity associated with any autoimmune disease. There are more than 80 autoimmune diseases known in the art, examples of which include multiple sclerosis (MS), insulin-dependent diabetes mellitus, systemic lupus erythematosus (SLE), psoriasis, autoimmune hepatitis, thyroiditis, insulitis, uveitis, orchitis, myasthenia gravis, idiopathic thrombocytopenic purpura, inflammatory bowel diseases (e.g., Crohn's disease and ulcerative colitis), encephalomyelitis, systemic autoimmune diseases (e.g., rheumatoid arthritis (RA), scleroderma, and juvenile arthritis).

Autoimmunity is “suppressed” if one or more symptoms of an autoimmune disease is reduced or alleviated in a mammal (e.g., a human) affected by an autoimmune disease. Improvement, worsening, regression, or progression of a symptom may be determined by any objective or subjective measure, many of which are known in the art. A person of ordinary skill in the art will appreciate that the symptoms of autoimmune diseases vary based on the disease and location of the abnormal immune response. Symptoms that are common to several autoimmune diseases include, for example, fatigue, muscle and/or joint pain, muscle weakness, fever, swollen glands, inflammation, susceptibility to infections, weight loss or gain, allergies, digestive problems, blood pressure changes, and vertigo.

The inventive cell population and methods can be used to decrease or suppress inflammation in the pancreas.

The inventive cell population and methods can be used to decrease or suppress the symptoms of AMD.

As used herein, the terms “treatment,” “treating,” and the like, refer to obtaining a desired pharmacologic and/or physiologic effect.

Preferably, the pharmacologic and/or physiologic effect is therapeutic, i.e., the effect partially or completely cures a disease and/or adverse symptom attributable to the disease. To this end, the inventive method comprises administering a “therapeutically effective amount” of the isolated IL-27-producing B-1a cell population. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the IL-27-producing B-1a cell population to elicit a desired response in the individual.

Alternatively, the pharmacologic and/or physiologic effect may be prophylactic, i.e., the effect completely or partially prevents an autoimmune disease or symptom thereof. In this respect, the inventive method comprises administering a “prophylactically effective amount” of the isolated IL-27-producing B-1a cell population to a mammal that is predisposed to, or otherwise at risk of developing, an autoimmune disease. A “prophylactically effective amount” refers to an amount effective, at dosages and for periods of time necessary, to achieve a desired prophylactic result (e.g., prevention of disease onset or prevention of disease flare-ups).

The isolated IL-27-producing B-1a cell population or composition comprising an isolated IL-27-producing B-1a cell population of the invention can be administered to a mammal using any suitable administration techniques, many of which are known in the art, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

When the inventive method comprises administering an isolated IL-27-producing B-1a cell population to a mammal, the isolated IL-27-producing B-1a cell population is administered to the mammal at a dose sufficient to induce the generation of B-cells that produce IL-27 and suppress autoimmunity in the mammal. Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

A typical amount of cells administered to a mammal (e.g., a human) can be, for example, in the range of 500,000 to 100 million cells, although amounts below or above this exemplary range can be suitable in the context of the invention. For example, the daily dose of cells can be about 500,000 to about 50 million cells (e.g., about 5 million cells, about 15 million cells, about 25 million cells, about 35 million cells, about 45 million cells, or a range defined by any two of the foregoing values), preferably about 10 million to about 100 million cells (e.g., about 20 million cells, about 30 million cells, about 40 million, about 60 million cells, about 70 million cells, about 80 million cells, about 90 million cells, or a range defined by any two of the foregoing values), more preferably about 10 million cells to about 50 million cells (e.g., about 12 million cells, about 25 million cells, about 35 million cells, about 45 million cells, or a range defined by any two of the foregoing values).

The invention can be utilized in combination with other existing therapies for autoimmune diseases. For example, the cell population of the invention can be administered in combination with immunosuppressive or immunomodulating agents or other anti-inflammatory agents for the treatment or prevention of an autoimmune disease, such as the autoimmune diseases disclosed herein. In this respect, the inventive method can be used in combination with disease-modifying anti-rheumatic drugs (DMARD) (e.g., gold salts, sulphasalazine, antimalarias, methotrexate, D-penicillamine, azathioprine, mycophenolic acid, cyclosporine A, tacrolimus, sirolimus, minocycline, leflunomide, and glucocorticoids), a calcineurin inhibitor (e.g., cyclosporin A or FK 506), a modulator of lymphocyte recirculation (e.g., FTY720 and FTY720 analogs), an mTOR inhibitor (e.g., rapamycin, 40-O-(2-hydroxyethyl)-rapamycin, CCI779, ABT578, AP23573, or TAFA-93), an ascomycin having immuno-suppressive properties (e.g., ABT-281, ASM981, etc.), corticosteroids, cyclophosphamide, azathioprene, methotrexate, leflunomide, mizoribine, mycophenolic acid, mycophenolate mofetil, 15-deoxyspergualine, or an immunosuppressive homologue, analogue or derivative thereof, immunosuppressive monoclonal antibodies (e.g., monoclonal antibodies to leukocyte receptors such as MHC, CD2, CD3, CD4, CD7, CD8, CD25, CD28, CD40. CD45, CD58, CD80, CD86, or their ligands), other immunomodulatory compounds, adhesion molecule inhibitors (e.g., LFA-1 antagonists, ICAM-1 or -3 antagonists, VCAM-4 antagonists, or VLA-4 antagonists), a chemotherapeutic agent (e.g., paclitaxel, gemcitabine, cisplatinum, doxorubicin, or 5-fluorouracil), anti-TNF agents (e.g. monoclonal antibodies to TNF such as infliximab, adalimumab, CDP870, or receptor constructs to TNF-RI or TNF-RII, such as ENBRELTM (Etanercept) or PEG-TNF-RI), blockers of proinflammatory cytokines, IL-1 blockers (e.g., KINERET™ (Anakinra) or IL-1 trap, AAL160, ACZ 885, and IL-6 blockers), chemokine blockers (e.g., inhibitors or activators of proteases), anti-IL-15 antibodies, anti-IL-6 antibodies, anti-CD20 antibodies, NSAIDs, and/or an anti-infectious agent.

The invention can be utilized in combination with administration of B-cells that produce interleukin-35 (IL-35). The B-cells that produce IL-35 (i35-Bregs) can be administered sequentially (before or after) or simultaneously with the cell population of the invention to a mammal.

Embodiments of the invention may be beneficial alone or in combination, with one or more other embodiments. Without limiting the foregoing description, certain non-limiting embodiments of the invention are provided below as embodiments numbered 1-26. As will be apparent to those of skill in the art upon reading this disclosure, each of the individually numbered embodiments may be used or combined with any of the preceding or following individually numbered embodiments. As such, the invention provides for all combinations of these embodiments and is not limited to combinations of embodiments explicitly provided below.

(1) An isolated population of mammal cells comprising about 75% or higher B-1a regulatory cells:

(a) expressing cell surface inhibitory receptors lymphocyte-activation gene 3 (LAG-3), programmed cell death protein 1 (PD-1), and C-X-C chemokine receptor type 4 (CXCR4); and (b) secreting interleukin-27 (IL-27).

(2) The population of mammal cells of embodiment (1), wherein the regulatory cells further express cell surface inhibitory receptor glucocorticoid-induced TNFR-related protein (GITR).

(3) The population of mammal cells of embodiment (1) or (2), wherein the regulatory cells further express cell surface inhibitory receptor OX40.

(4) The population of mammal cells of any one of embodiments (1)-(3), wherein the regulatory cells further express cell surface inhibitory receptor cytotoxic T-lymphocyte-associated protein 4 (CTLA4).

(5) A method of preparing the population of mammal cells of any one of embodiments (1)-(4), comprising
(a) isolating cluster of differentiation 5 positive (CDS+) expressing cells from a sample of mammal peripheral lymphoid tissue, mammal cord blood, mammal peritoneal fluid, induced pluripotent cells (iPSC), or mammal bone marrow using fluorescence-activated cell sorting (FACS) to provide isolated CDS+expressing cells;
(b) culturing the isolated CDS+expressing cells in a cell culture media to provide cultured cells;
(c) activating the cultured cells with a BCR (B cell receptor) or a TLR (Toll-like receptor) agonists to provide activated cells; and
(d) exposing the activated cells to IL-27.

(6) A method of suppressing the immune system in a mammal, the method comprising administering to a mammal the population of mammal cells of any one of embodiments (1)-(4).

(7) The method of embodiment (6), further comprising sequentially or simultaneously administering B-cells that produce interleukin-35 (IL-35) to the mammal.

(8) The method of embodiment (6) or (7), wherein administration treats a disease in the mammal.

(9) The method of any of one of embodiments (6)-(8), wherein the mammal has an autoimmune disease.

(10) The method of embodiment (9), wherein the autoimmune disease is a disease of the eye.

(11) The method of embodiment (9), wherein the autoimmune disease is a disease of the central nervous system.

(12) The method of embodiment (9), wherein the autoimmune disease is a disease of the brain.

(13) The method of embodiment (9), wherein the autoimmune disease is uveitis.

(14) The method of embodiment (9), wherein the autoimmune disease is encephalomyelitis.

(15) The method of any of one of embodiments (6)-(8), wherein the mammal has multiple sclerosis.

(16) The method of any of one of embodiments (6)-(8), wherein administration suppresses inflammation of the pancreas.

(17) The method of embodiment (6) or (7), wherein the mammal has received an allogeneic bone marrow or hematopoietic stem cell transplant.

(18) The method of embodiment (6) or (7), wherein the mammal has received an allogeneic solid organ transplant.

(19) The method of embodiment (17) or (18), wherein the mammal has graft-versus-host disease (GVHD).

(20) The method of any of one of embodiments (6)-(8), wherein the mammal has age-related macular degeneration (AMD).

(21) A method of treating a mammal with graft-versus-host disease, the method comprising administering the population of mammal cells of any one of embodiments (1)-(4) to a mammal with graft-versus-host disease.

(22) The method of embodiment (21), wherein the mammal received an allogeneic bone marrow or hematopoietic stem cell transplant prior to the administration of the population of mammal cells.

(23) The method of embodiment (21), wherein the mammal received an allogeneic solid organ transplant prior to the administration of the population of mammal cells.

(24) A method of preventing or reducing the severity of graft-versus-host disease in a mammal, the method comprising administering the population of mammal cells of any one of embodiments (1)-(4) to a mammal before the mammal receives an allogeneic transplant.

(25) The method of embodiment (24), wherein the allogeneic transplant is an allogeneic bone marrow or hematopoietic stem cell transplant.

(26) The method of embodiment (24), wherein the allogeneic transplant is an allogeneic solid organ transplant.

(27) A method of preventing or reducing the severity of graft-versus-host disease in a mammal, the method comprising (a) mixing the population of mammal cells of any one of embodiments (1)-(4) with a transplant material to form a transplant mixture; and (b) administering the transplant mixture to a mammal.

(28) The method of embodiment (27), wherein the transplant material comprises allogeneic lymphocytes.

(29) The population of mammal cells of any one of embodiments (1)-(4) or the method of any one of embodiments (5)-(28), wherein the mammal is a human.

### EXAMPLES

The following examples further illustrate the invention but, of course, should not be construed as in any way limiting its scope.

The following materials and procedures were used in Examples 1-5.

Mice and human PBMC and human cord blood CD19⁺ cells. Six- to 8-week-old C57BL/6J and IL-27RαKO mice were purchased from Jackson Laboratory (Bar Harbor, Maine). Female mice were used, and the mice were randomized for all the studies described. Human peripheral blood mononuclear cells (PBMC) were obtained from the National Institutes of Health (NIH) Blood Bank administered by the NIH Department of Transfusion

Medicine. Primary human umbilical cord blood CD19⁺ B cells were purchased from STEMCELL™ Technologies (Vancouver, Canada).

Isolation of mouse and human B cells. PBMC of normal human subjects were isolated from buffy coats by density gradient centrifugation by using a commercially available lymphocyte separation medium (Mediatech Inc., Manassas, Virginia). Human CD19⁺B cells were sorted using anti-CD19 antibody-conjugated magnetic beads (Miltenyl Biotec, Bergisch Gladbach, Germany). Mouse B2 cells were isolated from the spleen using B cell Isolation kit (130-090-862), CD19 MicroBeads (130-052-201), and Plasma Cell Isolation Kit (130-092-530) (all available from Miltenyl Biotec). B1 cells were isolated from the peritoneal cavity of C57BL/6J mice. Some of the mice were immunized with LPS in the presence or absence of IL-27. For the B-1a cells, the isolation was performed in a two-step procedure using the B-1a Cell Isolation Kit; Catalog # 130-097-413) as recommended by the manufacturer. Briefly, B-1a cells from the peritoneal cavity were negatively selected over a MACS™ magnetic cell column consisting of magnetic beads labeled with a cocktail of biotin-conjugated non-B-1a antibodies and the B-1a cells. The B-1a cells were then positively selected with magnetic beads conjugated with B-1a-specific antibodies.

Immunofluorescence Staining and Confocal Imaging Analysis. CD19⁺ B cells were activated in vitro for 48 h by stimulation with LPS or anti-CD40/anti-IgM antibodies in presence or absence of IL-27. The cells were fixed, blocked with 5% goat serum, and then incubated with fluorescence labelled anti-p28 (Invitrogen, Waltham, Mass.) or anti-Ebi3 antibody (Santa Cruz Biotechnology, Dallas, Texas). Cells were washed, incubated in ALEXA FLUOR™ 568-, ALEXA FLUOR™ 488-, or ALEXA FLUOR™ 647-conjugated secondary antibody (Invitrogen) containing 4′,6-diamidino-2-phenylindole (DAPI), and examined on a laser scanning confocal microscope (FV1000, Olympus Corporation, Tokyo, JP, or LSM700, Carl s AG) (see Oh et al., J. Biol. Chem., 287: 30436-30443 (2012)).

Experimental autoimmune uveitis (EAU). EAU was induced by active immunization of C57BL/6J and IL-27RaKO mice with interphotoreceptor retinoid binding protein (IRBP)₆₅₁₋₆₇₀-peptide in a 0.2 ml emulsion (1:1 v/v with complete Freund's adjuvant (CFA) containing Mycobacterium tuberculosis strain H37RA (2.5 mg/ml). Mice also received Bordetella pertussis toxin (1 μg/mouse) concurrently with immunization. Mice were treated by intraperitoneal injection of IL-27 (100 ng/mouse) or phosphate-buffered saline (PBS) on day −1 of immunization and every other day until day 12 post-immunization. For each study, 8 mice were used per group, and the mice were matched by age and sex. Clinical disease was established and scored by fundoscopy and histology (see Wang et al., Nat. Med., 20: 633-641 (2014), and Oh et al., I Immunol., 187: 3338-3346 (2011)). Eyes were examined for disease severity using a binocular microscope with coaxial illumination. Eyes for histology were enucleated 21 days post-immunization, fixed in 10% buffered formalin and serially sectioned in the vertical pupillary-optic nerve plane. All sections were stained with hematoxylin and eosin.

Fundoscopy. Funduscopic examinations were performed at day 10 to 21 after EAU induction. Briefly, following systemic administration of systemic anesthesia (intraperitoneal injection of ketamine (1.4 mg/mouse) and xylazine (0.12 mg/mouse)), the pupil was dilated by topical administration of 1% tropicamide ophthalmic solution (Alcon Inc., Fort Worth, Texas). The fundus image was captured using Micron III retinal imaging microscope (Phoenix Research Labs Pleasanton, California) for small rodent or a modified Karl Storz veterinary otoendoscope coupled with a Nikon D90 digital camera (see Oh et al., (2012), supra, and Paques et al., Invest Ophthalmol. Vis. Sci., 48: 2769-2774 (2007)). To avoid a subjective bias, evaluation of the fundus photographs was conducted without knowledge of the mouse identity by a masked observer. At least 6 images (2 posterior central retinal view, 4 peripheral retinal views) were taken from each eye by positioning the endoscope and viewing from superior, inferior, lateral and medial fields and each individual lesion was identified, mapped, and recorded. The clinical grading system for retinal inflammation was used (see Xu et al., Exp. Eye Res., 87: 319-326 (2008), and Chan et al., J. Autoimmun., 3: 247-255 (1990)).

Imaging mouse retina by Spectral-domain Optical Coherence Tomography (SD-OCT). Optical coherence tomography (OCT) is a noninvasive procedure that allows visualization of internal microstructure of various eye structures in living animals. An SD-OCT system with 820 nm center wavelength broadband light source (Bioptigen Inc., Morrisville, North Carolina) was used for in vivo non-contact imaging of eyes from control or EAU mice. Mice were anesthetized, and the pupils were dilated as described above. Mice were then immobilized using adjustable holder that could be rotated easily allowing for horizontal or vertical scan scanning. Each scan was performed at least twice, with realignment each time. The dimension of the scan (in depth and transverse extent) was adjusted until the optimal signal intensity and contrast was achieved. Retinal thickness was measured from the central retinal area of all images obtained from both horizontal and vertical scans from the same eye, using the system software, and averaged. A known method was used to determine the retinal thicknesses in the system software (see Gabriele et al., Invest. Ophthalmol. Vis. Sci., 52: 2250-2254 (2011)).

Electroretinogram (ERG). Before the ERG recordings, mice were dark-adapted overnight, and experiments were performed under dim red illumination. Mice were anesthetized with a single intraperitoneal injection of ketamine (1.4 mg/mouse) and xylazine (0.12 mg/mouse) and pupils were dilated with MIDRIN™ P containing of 0.5% tropicamide and 0.5% phenylephrine hydrochloride (Santen Pharmaceutical Co., Osaka, Japan). ERGs were recorded using an electroretinography console (Espion E2; Diagnosys LLC, Lowell, Massachusetts) that generated and controlled the light stimulus. Dark-adapted ERG was recorded with single-flash delivered in a ganzfeld dome with intensity of −4 to 1 log cd·s/m² delivered in 6 steps. Light-adapted ERG was obtained with a 20 cd/m² background, and light stimuli started at 0.3 to 30 cd·s/m² in 5 steps. Gonioscopic prism solution (Alcon Labs, Fort Worth, Tex.) was used to provide good electrical contact and to maintain corneal moisture. A reference electrode (gold wire) was placed in the mouth, and a ground electrode (subcutaneous stainless steel needle) was positioned at the base of the tail. Signals were differentially amplified and digitized at a rate of 1 kHz. Amplitudes of the major ERG components (a- and b-wave) were measured (Espion software; Diagnosys LLC, Lowell, Massachusetts) using automated and manual methods. Immediately after ERG recording, imaging of the fundus was performed as previously described.

Retinal cells isolation. To characterize inflammatory cells that cross the blood-retina barrier during EAU, mice were anesthetized and perfused with lx PBS. Enucleated eyes were put in Petri dishes containing culture medium (Roswell Park Memorial Institute medium (RPMI 1640)) for immediate isolation of the retina under a dissecting microscope. The eye was cut along the limbus of the eye and the lens and cornea were carefully removed. Then, the retina was peeled off and the attached optic nerve was removed before digesting the freshly isolated retina with collagenase (1 mg/ml) in RPMI 1640 medium containing 10 μg/ml DNase (Sigma-Aldrich, St. Louis, Mo.) for 2 hours at 37° C. During incubation, the cells were pipetted intermittently every 30 minutes and the digestion reactions was quenched with 5-10 fold volumes of 10% fetal bovine serum (FBS) in RPMI 1640 medium. The cells were washed twice in complete RPMI 1640 medium and the cells were counted using the VI-CELL' XR cell viability analyzer (Beckman Coulter, Brea, California).

Cell co-culture. Uveitogenic cells isolated from the lymph nodes and spleen of mice with EAU, B-1a, macrophages, and dendritic cells were isolated from EAU immunized mice on day 17. B-1a, macrophages, and dendritic cells were isolated by magnetic column beads (Miltenyi Biotech). Co-culture experiments were performed in a Trans-well system (Coming Incorporated, Corning, New York) in RPMI 1640 medium with 10% FBS. After seeding uveitogenic cells or B-1a cells (5 x10⁵) in the bottom well, macrophages or dendritic cells (5x10⁵) were seeded in the upper chamber (pore size: 0.4 p.m) and re-stimulated with IRBP₆₅₁₋₆₇₀ (20 μg/ml). Cells were collected for analysis with flow cytometry and thymidine incorporation assay after 72 h of the co-culture. For functional analysis of human B-1a cells, CD19⁺CD20^{+CD}27⁺CD43⁺B1 cells from healthy controls were purified by cell sorting and stimulated with anti-CD40 (10 μg/m1) plus anti-IgM (5 μg/m1) in the presence or absence of rhlL-27 (100 ng/ml) for 72 h.

Experimental Autoimmune Encephalomyelitis (EAE). EAE was induced by subcutaneous immunization with 200 pg myelin oligodendrocyte glycoprotein peptide 35-55 (MOG35-55) (Sigma-Aldrich) in CFA emulsion, containing 2.5 mg/ml of heat killed, pulverized Mycobacterium tuberculosis strain H37RA. The mice also received two doses of 0.3 μg Bordetella pertussis toxin (Sigma-Aldrich) on day 0, and day 2 post-immunization by intraperitoneal (i.p.) injection in 100 μl of RPMI 1640 medium containing 0.1% normal mouse serum. Some mice received IL-27 (100 ng/mouse) concurrently with immunization with MOG₃₅₋₅₅ and every other day until day 12 post-immunization. The control or IL-27-treated group (n=12) was euthanized 17 days post-immunization. The mice were monitored, and disease severity was assessed daily by a masked observer. Clinical signs of EAE were graded according to the following scale: 0, No clinical symptoms; 1, clumsiness, incontinence or atonic bladder, flaccid tail; 2, mild paraparesis (trouble initiating movement); 3, moderate paraparesis (hind limb weakness); 4, complete front and hind limb paralysis; 5, moribund state (see Liu et al., J. Immunol., 180: 6070-6076 (2008)). Spinal cords and brains were harvested 17 days post-immunization and stained with hematoxylin and eosin (H&E).

For adoptive transfer studies, mice with EAE, treated with or without IL-27, were sacrificed on day 10 post-immunization and used as donors in passive induction of EAE by adoptive transfer of encephalitogenic cells. Spleen cells were isolated, stimulated with MOG35-55 peptide (20 μg/ml) and anti-CD40 (10 μg/ml) antibody for 3 days in the presence or absence of IL-27 and transferred intravenously (i.v.) to naive syngeneic recipient mice (10×10⁶ cells/mouse; n=12). Twenty days after adoptive cell transfer, disease was assessed and brain or spinal cord tissue was collected from recipient mice, fixed in 10% buffered formalin, and sectioned for histopathological examination. Central Nervous System (CNS) infiltrates were collected from the brain and spinal cord and lymphocytes/mononuclear cells were isolated by collagenase digestion followed by percoll gradient for analysis.

Adoptive transfer of B-1a cells. B-1a cells were isolated from the peritoneal cavity of donor mice and sorted using magnetic beads. The B-1a cells were cultured in complete RPMI 1640 with LPS (1 μg/ml) for 48 h, washed (2×) to remove residual LPS and adoptively transferred (5×10⁵) into C57BL/6J and IL-27RaKO mice.

In vivo model of LPS-induced inflammation. C57BL/6J mice were injected with LPS (50 μg/mouse) and some mice received IL-27 (100 ng/mouse) 1 h before LPS injection by i.v. route. The mice in the control and IL-27-treated group (n=5) were euthanized 24 h post-injection and spleen cells were subjected to fluorescence-activated cell sorter (FACS) analysis.

Proliferation assay. Uveitogenic cells or B-1a cells were harvested from IRBP immunized C57BL/6J or IL-27RaKO mice at day 17 post-immunization. The cells were re-stimulated in vitro with IRBP peptide for 72 h in the presence or absence of B-1a, dendritic cell, and macrophages. For in vitro studies, CD19⁺B cells were stimulated with anti-CD40 antibodies (10 μg/ml) and anti-IgM antibodies (5 μg/ml) in the presence or absence of IL-27. Cells were pulsed with ³H-thymidine (0.5 μCOO μl/well) for the last 24 h in culture. Presented data are mean CPM±S.E.M. of responses of 5 replicate cultures.

Detection of Cytokine-expressing Lymphocytes by FACS. CD19⁺ B cells (>98%) were stimulated with LPS (2 μg/ml) or activated with anti-CD40 antibodies (10 μg/m1) and anti-IgM antibodies (5 μg/ml) as described above. For intracellular cytokine detection, cells were re-stimulated for 5 h with phorbol myristate acetate (PMA) (50 ng/ml)/ionomycin (500 ng/ml). GOLGIPLUG™ (BD Pharmingen, San Diego, California) was added in the last three hours and intracellular cytokine staining was performed using BD CYTOFIX/CYTOPERM™ kit as recommended (BD Pharmingen). FACS analysis was performed on a MACSQUANT™ analyzer (Miltenyi Biotec) using protein-specific monoclonal antibodies and corresponding isotype control antibodies (BD Pharmingen) (see Amadi-Obi et al., Nat. Med., 13: 711-718 (2007), and Wang et al., Nat. Med., 20: 633-641 (2014)). FACS analysis was performed on samples stained with monoclonal antibodies conjugated with fluorescent dyes (including CD19, CD20, CD24, CD27, CD38, CD43, CD138, and CD11b). Cells were color compensated and quadrant gates were set using isotype controls with less than 0.3% background. Live cells were subjected to side-scatter (SSC) and forward scatter (FSC) analysis.

Characterization of Regulatory B (Breg) and T (Treg) cells. Primary B cells isolated from the brain, spinal cord, retina, peritoneal cavity, blood, spleen, or draining lymph node (LN) of unimmunized, EAE or EAU mice were sorted for CD19⁺cells and used for surface and intracellular FACS analysis. Some cells were reactivated with LPS, IRBP₆₅₁₋₆₇₀-peptide and anti-CD40 antibody, MOG35-55-peptide and anti-CD40 (see Wang et al., Nat. Med., 20: 633-641 (2014), and Choi et al., Front Immunol., 8: 1258 (2017)). For intracellular cytokine detection, cells were re-stimulated for 5 h with PMA (50 ng/ml) and ionomycin (500 ng/ml). GOLGIPLUG™ (BD Pharmingen) was added in the last hour, and intracellular cytokine staining was performed using the BD BD CYTOFIX/CYTOPERM™ kit as recommended (BD Pharmingen). FACS analysis was performed on a MACSQUANT™ analyzer (Miltenyi Biotec) using protein-specific monoclonal antibodies and corresponding isotype control antibodies (BD Pharmingen) as previously described by using protein-specific monoclonal antibodies and corresponding isotype control antibodies (BD Pharmingen). Dead cells were stained with dead cell exclusion dye (Fixable Viability Dye EFLUOR™ 450, Thermo Fisher Scientific), and live cells were subjected to side-scatter (SSC) and forward-scatter (FSC) analyses. Breg and Treg cells were characterized by analysis of the expression of CD4, CD19, CDS, CD27, CD38, CD138, B220, CD1d, IL-10, p28, p35 or Ebi3. FACS analysis was performed on cells stained with monoclonal antibodies conjugated with fluorescent dyes, dead cells were excluded, and each tube of cells was color-compensated. Quadrant gates were set using isotype controls with less than 0.5% background. CRISPR/Cas9-mediated gene deletion. sgRNA was generated and cloned into lentiCRISPR v2, pMD2.G using a known technique (see Sanjana et al., Nat. Methods, 11: 783-784 (2014)). The sgRNAs were selected by CRISPRSCAN, an online tool racking sgRNA sites by their on-target binding efficiency and probabilities of off-target hits. For IL-27, three sgRNAs were selected and cloned into a Lentiviral vector carrying the SpCas9 sgRNA scaffold driven by the U6 promoter. The sgRNA sequences were: sgp28 targeting site 1, 5′-GCTTCCTCGCTACCACACT-3′ (SEQ ID NO: 1), site 2; 5′-GGGCCATGAGGCTGGAT CTC-3′(SEQ ID NO: 2); site 3 5′-GATGGTATCCCAGGGGCAGG-3′(SEQ ID NO: 3). For Ebi3 targeting, the same Lentiviral vector was used for cloning of three sgRNAs: site 1; 5′-GTCGGGGATGGTGCATCGGG-3′(SEQ ID NO: 4); site 2 5′-TCTCTGATGGGTCACTAACT-3′(SEQ ID NO: 5); site 3 5′-CAGGAGCAGTCCACGGCCAC-3′(SEQ ID NO: 6). For deletion of IL-27, purified B-1a cells or macrophages were transduced with lentiviral clones expressing the sgRNAs. Two days after infection, cells were activated with LPS for 48 h and analyzed by FACS or ELISA.

Detection of cytokine secretion by ELISA. CD19⁺B cells or B-1a cells were activated in vitro in presence or absence of LPS, anti-CD40 plus anti-IgM and/or IL-27. Supernatants were collected after 48 h in culture. IL-27 and IL-35 were quantified using mouse IL-27- or IL-35-specific heterodimeric ELISA kit (BioLegend, San Diego, California). IL-17 or IL-10 were quantified using kits from R&D systems as recommended by manufacturer.

RNA extraction, NanoString analysis, and PCR. Total RNA was isolated from the peritoneal cavity or spleen using RNEASY™ plus mini kit (Qiagen, Hilden, Germany). cDNA synthesis, RT-PCR and qPCR analyses were performed according to known techniques (see Amadi-Obi et al., Nat. Med., 13: 711-718 (2007)). Each gene-specific primer pair used for RT-PCR analysis spans at least an intron. The following primers and probes used for qPCR were purchased from Applied Biosystems (Foster City, Calif.): IRF8 (Mm_00492567), IRF4 (Mm_00516431), BCL6 (Mm_00477633), Blimpl (Mm_00476128), Pax5 (Mm_00435501), Lag-3 (Mm_01185091), PD-1 (Mm_00435532), IL-27 (Mm_004461162), IL-12a (Mm_00434169), IL-10 (Mm_00439614), IL-27Ra (Mm_00497259), p21 (Mm 00817699), p27 (Mm_00438168), Cdkl (Mm 00772472), Cdk2 (Mm_00443947), Cdk4 (Mm_00726334), and mRNA expression was normalized to the levels of GADPH (Mm_99999915) genes. For NanoString nCounter analysis, 100 ng total of RNA per sample was used. A custom nCounter Gene Expression CodeSet immunology panel was used. Data was normalized using housekeeping genes and analyzed with nSolver Analysis software, version 3.

Immunoprecipitation and immunoblotting. Whole cell lysates were prepared according to a known technique (see Li et al., Invest. Ophthalmol. Vis. Sci., 40: 976-982 (1999)). Cleared lysates or cellular supernatants were immunoprecipitated with antibody that was pre-coupled to protein G-sepharose beads according to a known technique (see Oh et al., J. Biol. Chem., 286: 30888-30897 (2014)). Immunoprecipitates were resolved by sodium dodecyl sulfate (SDS)-polyacrylamide gel electrophoresis (PAGE) and blots were probed with specific antibodies. The following antibodies were used for immunoprecipitation and/or Western blotting: p28 (Invitrogen), Ebi3, and β-actin (Santa Cruz Biotechnology). Pre-immune serum was used in parallel as controls and signals were detected with HRP conjugated-secondary F(ab')2 (Zymed Labs, San Francisco, Calif.) using ECL system (Amersham, Arlington Heights, Ill).

Western blotting analysis. Preparation of whole cell lysates and performance of Western blot analysis were performed according to known techniques (see Wang et al., Nat. Med., 20: 633-641 (2014) and Egwuagu et al., J. Immunol., 168: 3181-3187 (2002)). Cell extracts (20-40 μg/lane) were fractionated on 10% gradient SDS-PAGE in reduced condition and Western blot analysis was performed using antibodies specific to pSTAT1, pSTAT3, STAT1, STAT3, p28, p35, Ebi3, IL-27Ra, GP130, IRF8 or β-Actin (Santa Cruz Biotechnology and Cell Signaling Technology, Danvers, Mass.). Pre-immune serum was used in parallel as controls and signals were detected with HRP-conjugated secondary F(ab′)₂ Ab (Zymed Laboratories) using the ECL-PLUS system (Amersham). Each Western blotting analysis was repeated at least three times.

Chromatin Immunoprecipitation (ChIP) analyses. ChIP assays were performed using EZ-CHIP™ chromatin immunoprecipitation kits (Millipore Sigma, Darmstadt, Germany). B cells were activated with LPS in presence or absence of IL-27 and DNA-protein complexes were cross-linked for 10 min by addition of fresh formaldehyde (Sigma-Aldrich) to the culture medium at a final concentration of 1%, followed by quenching in 135 mM glycine. The cells were then washed in cold PBS (2×), lysed (EZ-CHIPTM lysis buffer) and sonicated (5×) in 15s bursts (output 5 on Sonic Dismembrator Model 1000, Thermo Fisher Scientific). Lysates were then cleared with Protein G-agarose for 1 h, pelleted, and incubated overnight with control IgG or anti-STAT1 or STAT3 antibody (Cell Signaling Technology). Prior to antibody incubation, input samples were removed from the lysate and stored at −80 ° C. until extraction. Immunoprecipitation was performed according to the manufacturer's instructions (EZ-CHIP™). The immunoprecipitated and input DNA were subjected to PCR and qPCR using primers to detect STAT1 and STAT3 binding activities. The primers (5′-CTGAAACCCCAGCTTCCTGCCA-3′ (SEQ ID NO: 7) and 5′-CATCTCCTGGGTAGGGGGGTCTTATACT-3′(SEQ ID NO: 8)) for IL-27p28 gene promoter are from -134 to -303 with STAT binding motif GGAAGGGAAATTACGTT (SEQ ID NO: 9), while the primers (5′-CTGATTCTGTCTCTGTTTCTCTCAGTT-3′ (SEQ ID NO: 10) and 5′-GTGGGGAAAGGCCTTGAGGTAGA-3′ (SEQ ID NO: 11)) for EBI3 gene promoter region are from -1 to -150 with STAT binding motif CCTCAAGGCCTTTCC (SEQ ID NO: 12).

Electrophoretic mobility shift assay (EMSA). EMSA was performed according to well-known procedures (see Yu et al., J. Immunol., 157: 126-137 (1996)). The double stranded oligonucleotides containing motifs from the AP1-IRF-1 composite elements (AICE) 5′TGAnTCA/GAAA-3′ (SEQ ID NO: 13) were labeled by a fill-in reaction using Klenow polymerase (New England BioLabs, Beverly, Mass.) with [alpha-P³²]dATP or (alpha-32P)dGTP (3000 Ci/mmol) (PerkinElmer Inc., Waltham, Mass.). Sorted CD19⁺ B cells were stimulated with LPS (1 μg/ml) in the presence or absence IL-27 (20 μg/ml) for three days and nuclear extracts were prepared in buffer containing the following protease inhibitors: 2 μM leupeptin, 2 μM pepstatin, 0.1 μM aprotinin, 1 mM [4-(2-aminoethyl)benzenesulfonyl fluoride, hydrochloride], 0.5 mM phenylmethyl-sulfonyl fluoride, and 1 μM E-64 [N-(N-1-trans-carboxyoxiran-2-carbonyl)-1-leucyflagmatine according to known procedures (see Yu et al., J. Immunol., 157: 126-137 (1996)). Protein levels were determined by the BCA method as recommended, and extracts were stored at —70 ° C. until use. DNA-protein binding reaction was performed in a 20-p.1 mixture containing 5 μg nuclear protein and 1 μg double-stranded poly(d1:C) (Boehringer Mannheim, Barcelona, Spain), 12 mM HEPES (pH 7.9), 60 mM KCI, 0.5 mM DTT, 12% glycerol, 2.5 mM MgCl. After a 15 min incubation on ice, samples were further incubated with 1 μl P³²-labeled probe (15,000 cpm) at room temperature for 20 min and fractionated on 5% native polyacrylamide gel in 0.25×Tris-borate-EDTA buffer. For super-shift analysis, before the addition of ³²P-labeled probes, extracts were pre-incubated with 1 μl antibodies specific to basic leucine zipper transcription factor (BATF) (Cell Signaling Technology), Jun B, Jun D, IRF-4, IRF-8 or IRF-1 (Santa Cruz Biotechnology).

Proximity ligation assay. The Proximity Ligation Assays (PLA) were performed with Duolink PLA kit (Sigma Aldrich, St. Louis, Mo.). Activated B cells were attached to slides, blocked for 1 h in blocking solution and then incubated overnight with primary mouse anti-p28 (rabbit) and anti-Ebi3 (mouse). A pair of oligonucleotide-labeled secondary Abs (PLA probes) that bind to the primary Abs were then added, incubated for 1 h, and then ligation solution containing hybridizing connector oligos was added. PLA probes in close proximity (within 40nm) then interacted and ligated to the connector oligos. The resulting closed, circular DNA template was amplified by DNA polymerase. Complementary detection oligos coupled to fluorochromes hybridized to repeating sequences in the amplicons and p28:Ebi3 heterodimers are then detected as discrete fluorescent spots by confocal microscopy (LSM 700, Carl Zeiss AG, Oberkochen, Germany).

RNA-Seq and Analysis. For RNA-Seq, mRNA was isolated by oligo-dT beads and a library was prepared using the standard Illumina, Inc. library protocol (kit RS-122-2101 TruSeq Stranded mRNA LT Sample prep kit, Illumina, Inc., San Diego, Calif.). Libraries were sequenced on the NovaSeq 6000 system (Illumina, Inc.). The relative abundances of genes were measured in Read Count using StringTie. The statistical analysis was performed to find differentially expressed genes using the estimates of abundances for each gene in samples. Genes with one more than zeroed Read Count values in the samples were excluded. To facilitate loge transformation, 1 was added to each Read Count value of filtered genes. Filtered data were loge-transformed and subjected to the trimmed mean of M-values (TMM) normalization method. Statistical significance of the differential expression data was determined using exact t-test using edgeR and fold change in which the null hypothesis was that no difference exists among groups. P values were adjusted for multiple testing using the false discovery rate (FDR) correction of Benjamin and Hochberg. For heatmaps, the R package heatmap and Broad institute tool Morpheus was used to normalized counts. Hierarchical clustering analysis was performed using complete linkage and Euclidean distance as a measure of similarity to display the expression patterns of differentially expressed transcripts which are satisfied with |fold change|≥2 and independent t-test raw p <0.05.

Statistical analysis. Graphs were plotted and analyzed using GraphPad Prism 7.0, two-tailed unpaired Student's t test, non-parametric Mann-Whitney U-test or One way ANOVA depending on the experiments. Probability values of <0.05 were considered statistically significant. Some data are presented as mean +SEM. Asterisks denote p value as follows: *P<0.05, **P<0.01, ***P<0.001, and ****P<0.0001.

Sample sizes are indicated in figures or figure legends and refer to number of animals. In vitro assays using human cord blood or PBMC were repeated independently using cells from at least three unrelated donors. Results shown represent at least three independent experiments as noted in the legends. Optical coherence tomography, ERG and confocal image analyses were performed blindly. EAE and EAU scoring were performed by masked investigators. Essential immunotherapeutic effects of i27-Breg in EAU was validated and recapitulated in the EAE model. Mice were age/sex matched and randomized, consisting of equal numbers of males and females.

### EXAMPLE 1

This example demonstrated that peritoneal B1 cells secrete IL-27 (i27-Bregs) and activation of i27-Bregs during inflammation triggers their exodus into secondary lymphoid tissues.

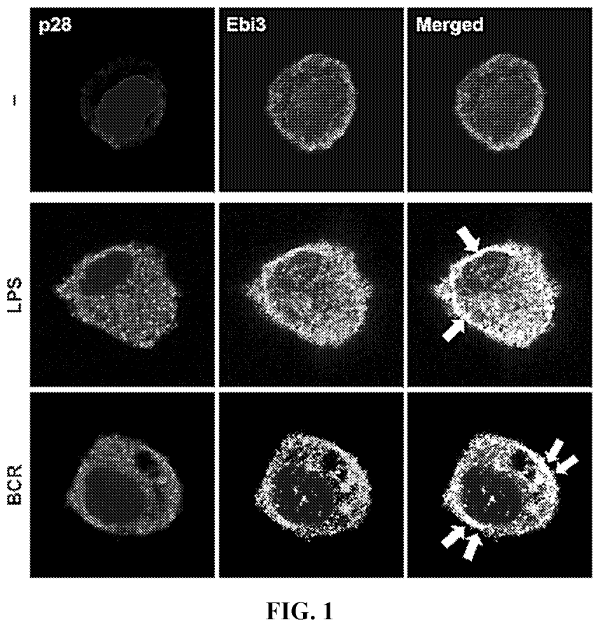

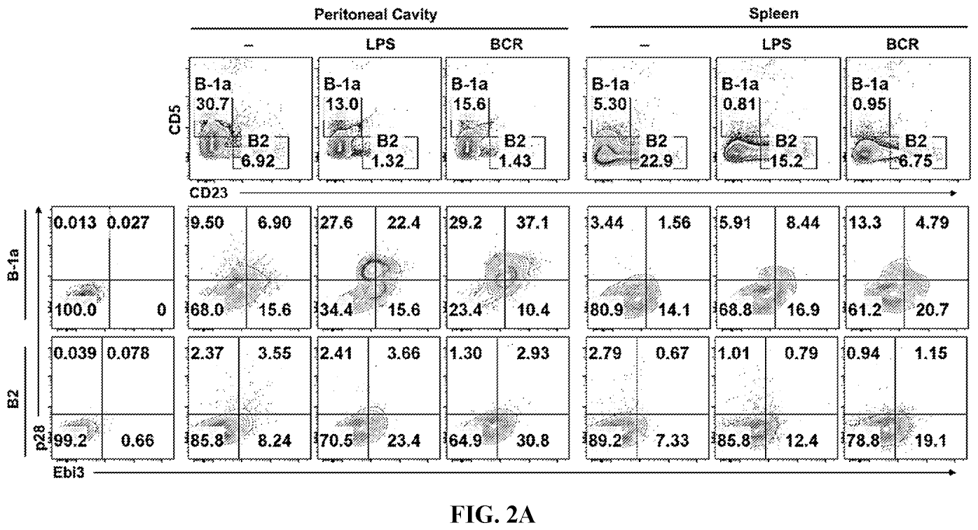

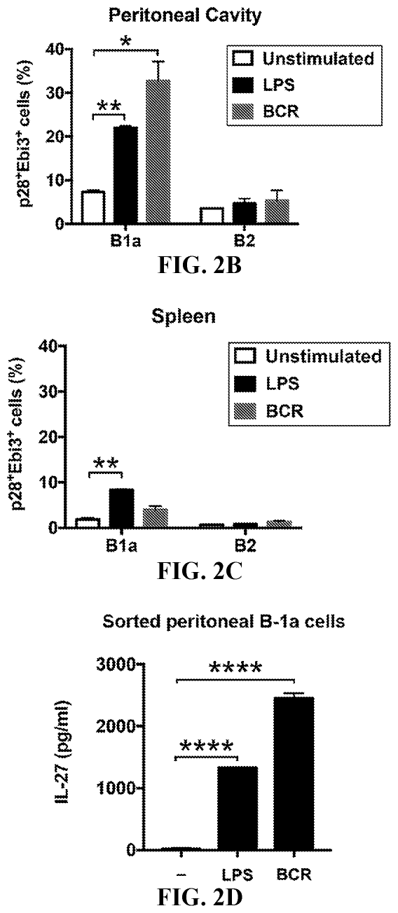

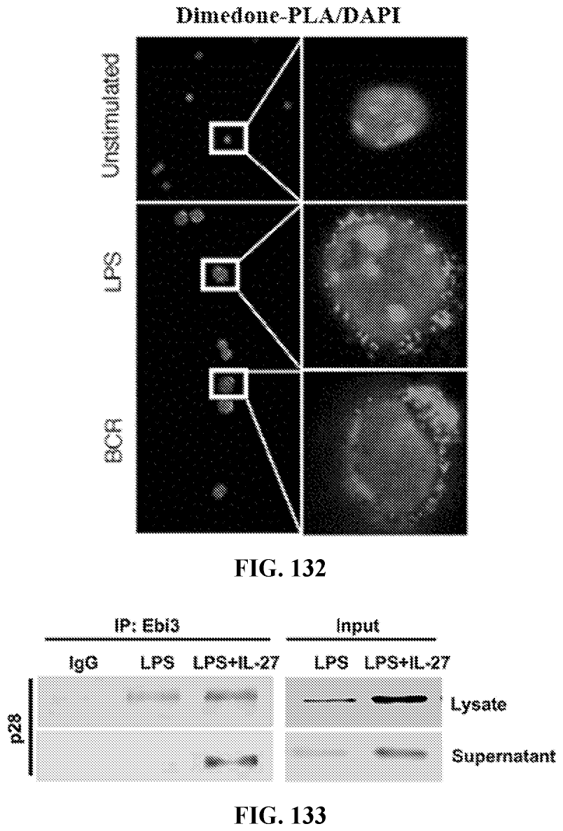

Immunohistochemical/confocal microscopy co-localized p28 and Ebi3 expression on activated mouse CD19⁺ B-cells (FIG. 1, white arrows), indicating that B-lymphocytes produce IL-27. B1-lymphocytes (B-1a and B-1b) are innate B-cells localized primarily in peritoneal cavity while B2 are conventional Ag-specific B-cells in spleen. Flow cytometric intracellular cytokine staining of activated B-cells in the mouse peritoneal cavity or spleen revealed that both these developmentally and functionally distinct B-cell lineages can produce IL-27 (FIG. 2). However, regardless of the activating stimulus or source of the B-cells, B-1a cells are the major producers of IL-27 (FIG. 2A) and production of IL-27 by B-1a cells was confirmed by ELISA (FIG. 2D). Reciprocal IP/Western analyses detected co-expression of p28 and Ebi3 in lysates and supernatant of activated B-1a cells, providing further evidence that B-1a cells do indeed secrete the heterodimeric IL-27(p28/Ebi3). PLA further demonstrated physical interaction between p28 and Ebi3 (FIG. 132), providing direct evidence that B cells secrete the heterodimeric IL-27 cytokine. Reciprocal immuno-precipitation and Western blot (IP/Western) analysis of whole cell extracts or supernatant of activated B-1a cells detected co-expression of p28 and Ebi3 (FIG. 133), further confirming that B cells secrete heterodimeric IL-27.

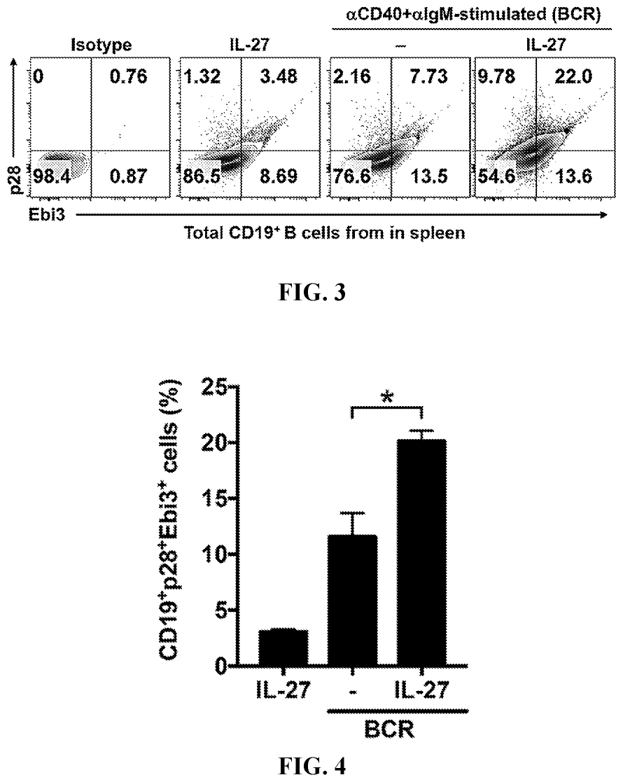

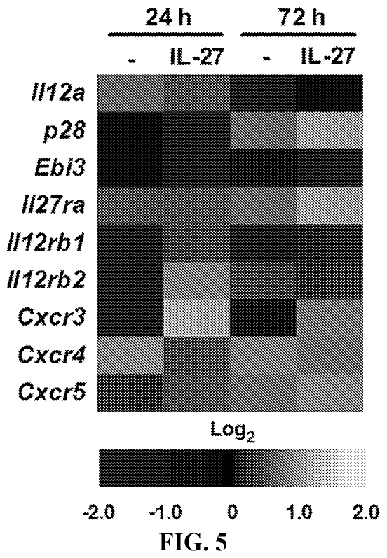

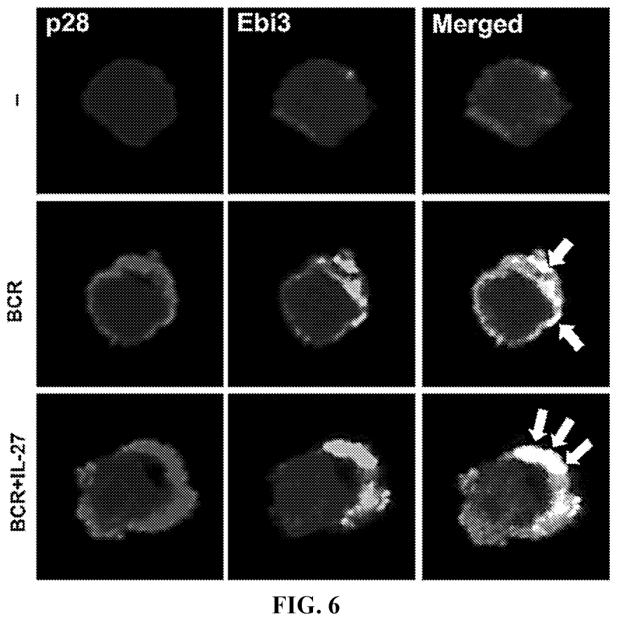

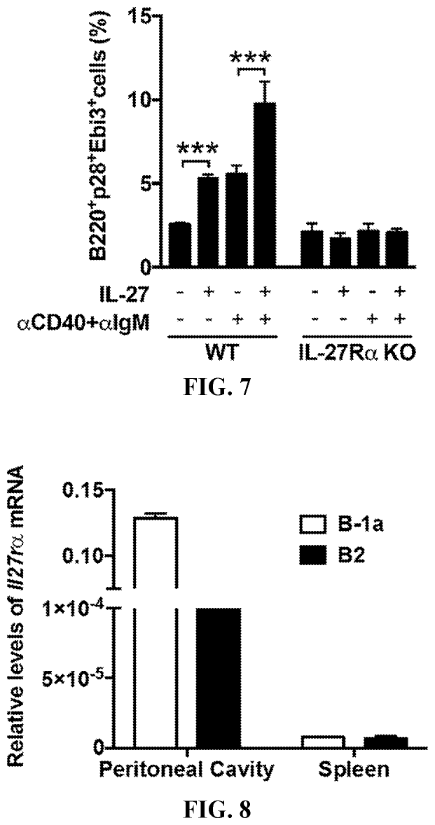

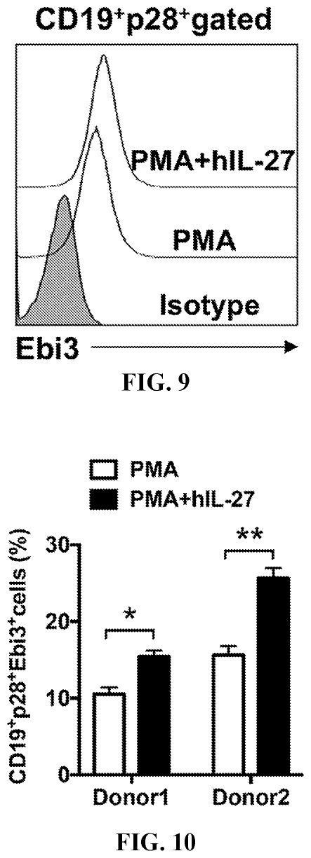

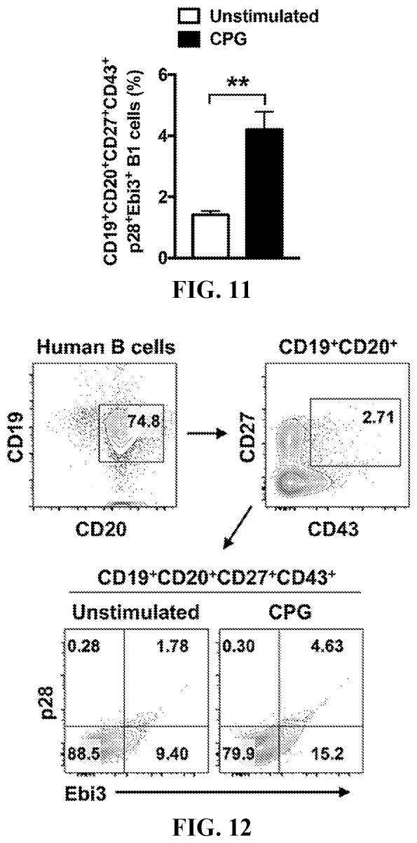

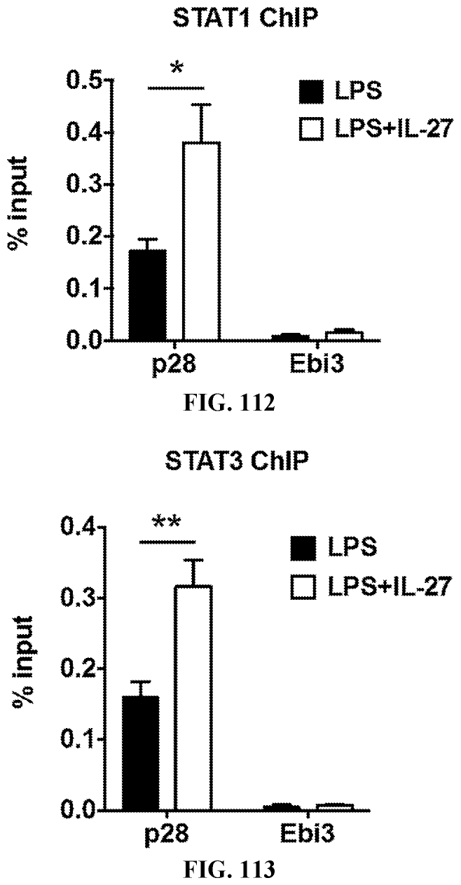

FACS analysis of activated B-cells revealed a discrete population of IL-27-producing B-cells (about 7.73%) that increased (2.85-fold) in response to IL-27 (FIG. 3), suggesting that exposure to IL-27 can induce expansion of i27-Bregs. NanoString RNA analysis (FIG. 5) and Western blotting also showed that BCR/IL-27 synergistically upregulated expression of IL-27 subunit p28, IL-27Ra and altered the pattern of chemokine receptors expression (FIG. 5). Immunohistochemical/confocal microscopy analysis also detected upregulated expression of IL-27 (white arrows) by B-cells in response to IL-27/BCR-signaling (FIG. 6), suggesting that BCR and IL-27 signals may be required for optimal expansion of i27-Bregs. Furthermore, chromatin immunoprecipitation assay demonstrated that IL-27 mediated its effects by inducing the binding of activated STAT1 and STAT3 to 1127a proximal promoter (FIGS. 112 and 113). While BCR/IL-27-induced signals promoted expansion of IL-27-producing cells, BCR/IL-27-induced signals could not expand these cells in cultures of B-cells lacking IL-27 receptor (IL-27RaKO) (FIG. 7), underscoring the requirement for IL-27 signals for generation of the IL-27-producing B-cells. Consistent with the requirement of IL-27 for autocrine expansion of IL-27-producing B-1a cells is the observation that IL-27 up-regulates IL-27Ra expression in B1 cells (FIG. 8). Importantly, in the context of applicability of IL-27-producing B-cells for immunotherapy, innate-like human B1 cells were found to also produce IL-27 (FIGS. 9-11).

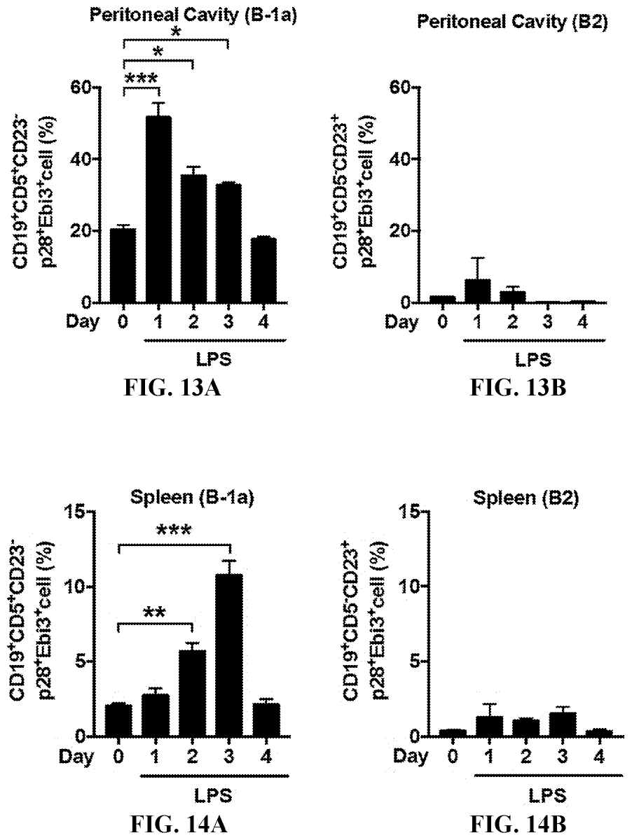

To investigate whether B-cells can produce IL-27 in vivo, C57BL/6J mice were injected (i.v) with LPS, and the percentage of IL-27-producing B-1a or B2 cells in the peritoneal cavity or spleen was determined. As many as about 19.4% of B-1a cells in the peritoneal cavity of PBS-treated mice were producing IL-27 at the 24 h time-point while the percentage of these cells increased to ˜55.6% in mice injected with LPS (FIGS. 13A-14B). The rapid kinetics of this response indicates mobilization, rather than proliferation. Interestingly, the percentage of IL-27-secreting B-1a cells progressively declined with time in the peritoneal cavity and eventually returned to basal level by day 4 of inflammation (FIGS. 13A-14B). Similar analysis revealed a different pattern of recruitment of IL-27-producing B-1a cells into the spleen. From day 1 after injection with LPS, the percentage of B-1a cells recruited into the spleen progressively increased from 2.01%, reached a peak of 8.8% by day 3, and then returned to basal level on day 4 of the inflammation (FIG. 14B). Note that IL-27-producing B2 cells in the spleen or peritoneal cavity never exceeded 2% (FIGS. 13B and 14B). These results indicate that injection of LPS induced rapid increase in IL-27-producing B-1a cells followed by their egress from peritoneal cavity, and these events correlated temporarily with the subsequent recruitment of B-1a cells into the spleen.

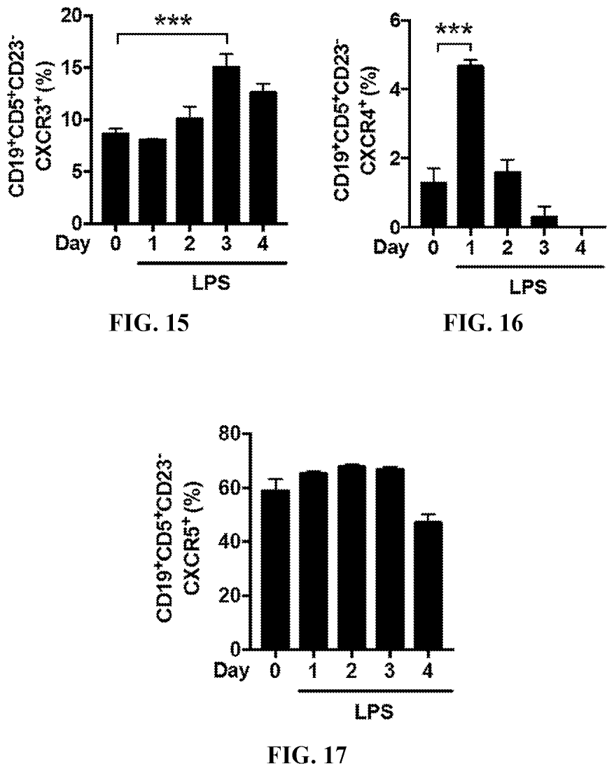

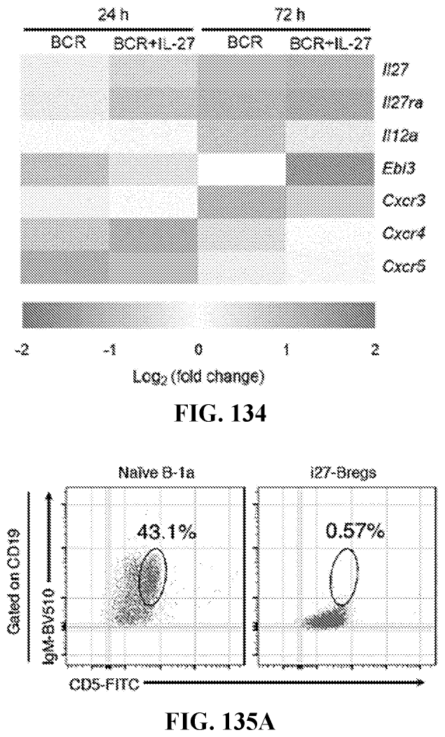

The data show a time-dependent increase of CXCR3- and CXCRS-expressing B-1a cells in the spleen which coincided temporally with significant decrease of CXCR4-expressing B-1a cells in the peritoneal cavity (FIGS. 15-17). These results are in line with the NanoString data (FIG. 5) which showed upregulation of Cxcr5 and downregulation of Cxcr4 transcription by B-1a cells in response to IL-27 (FIGS. 5 and 134).

Taken together, these observations suggest that differential regulation of chemokine receptors expression by B-1a cells in response to IL-27 promotes egress of B-1a cells from the peritoneal cavity and their subsequent trafficking to the spleen.

### EXAMPLE 2

This example demonstrated that IL-27-producing B-1a cells (i27-Bregs) confer protection from severe uveitis.

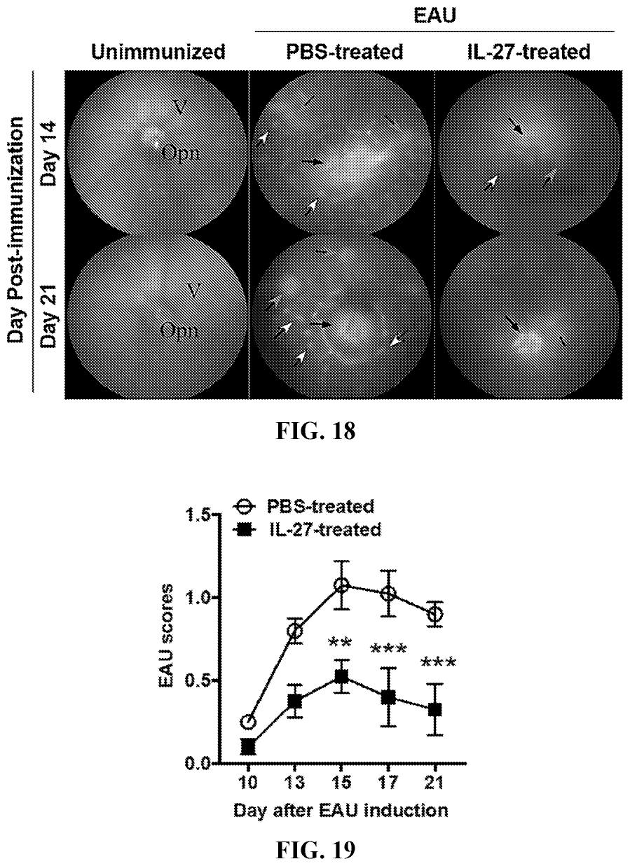

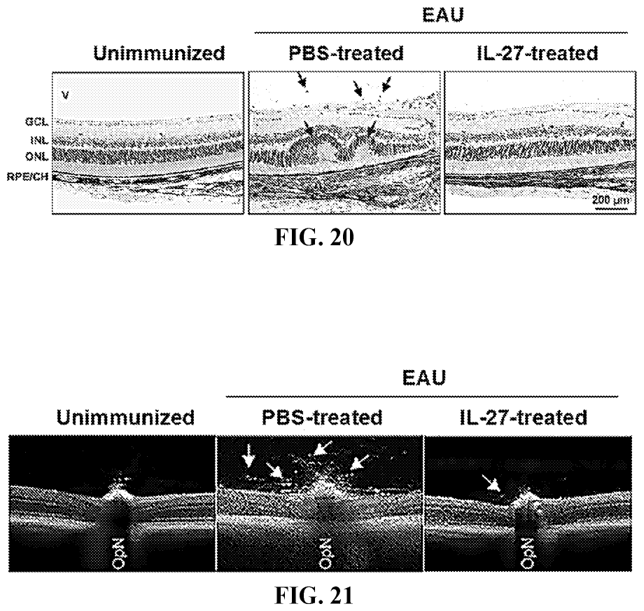

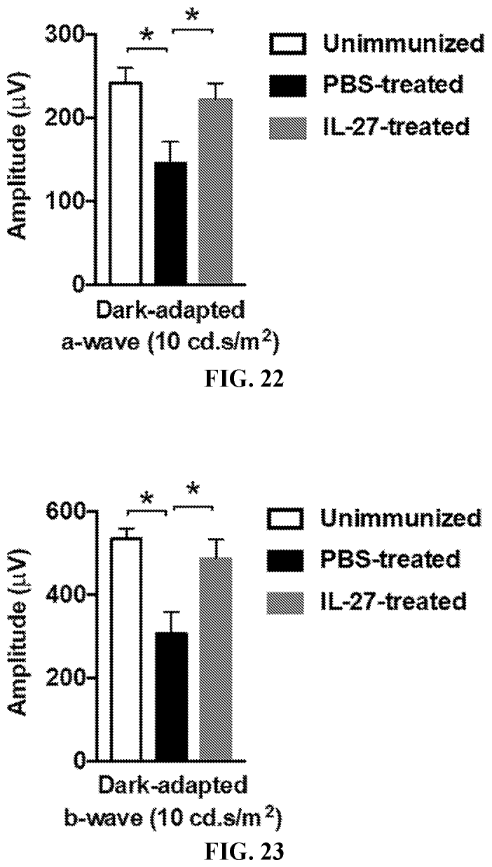

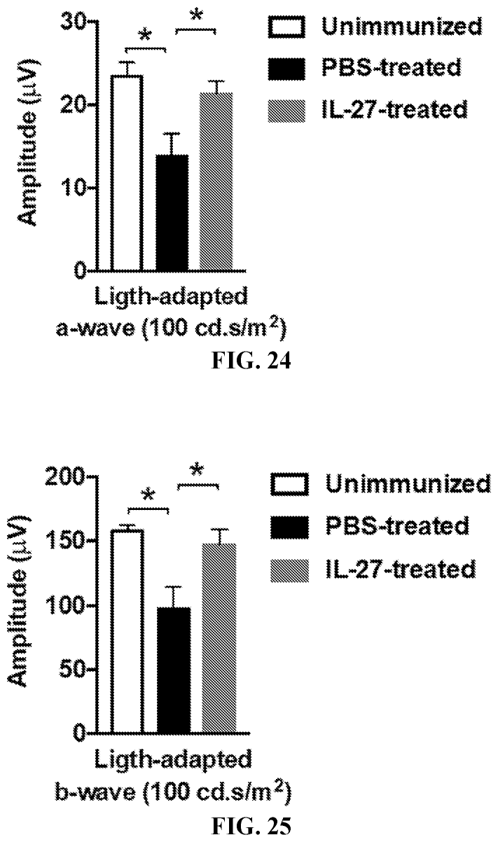

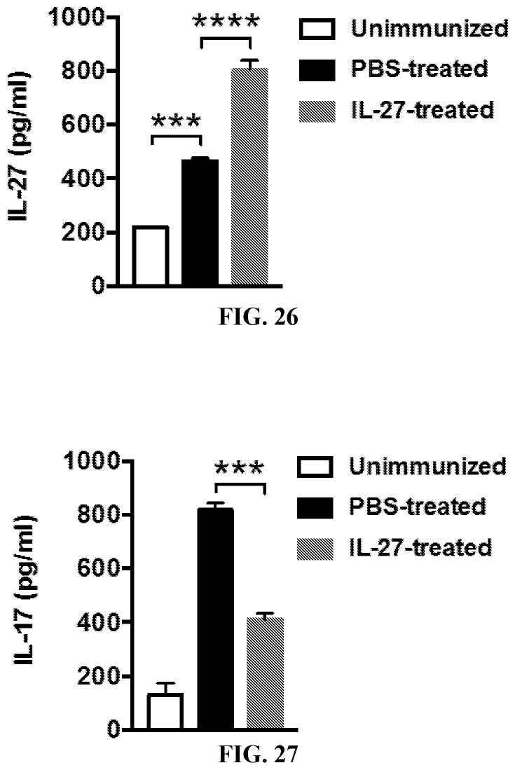

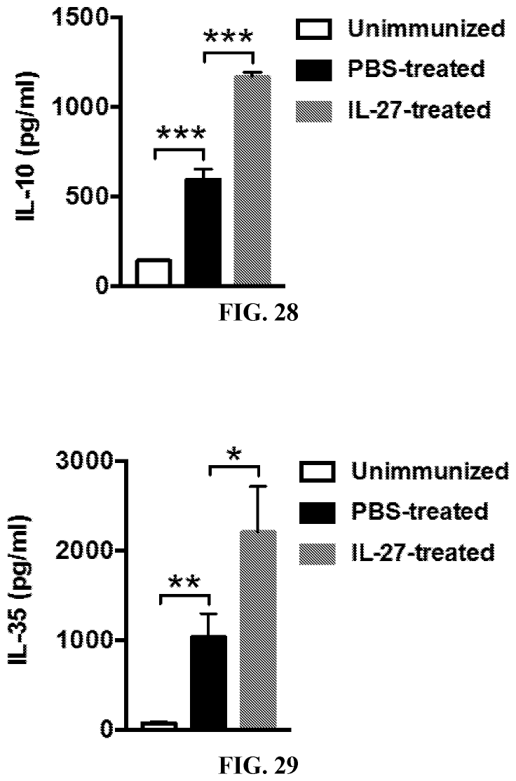

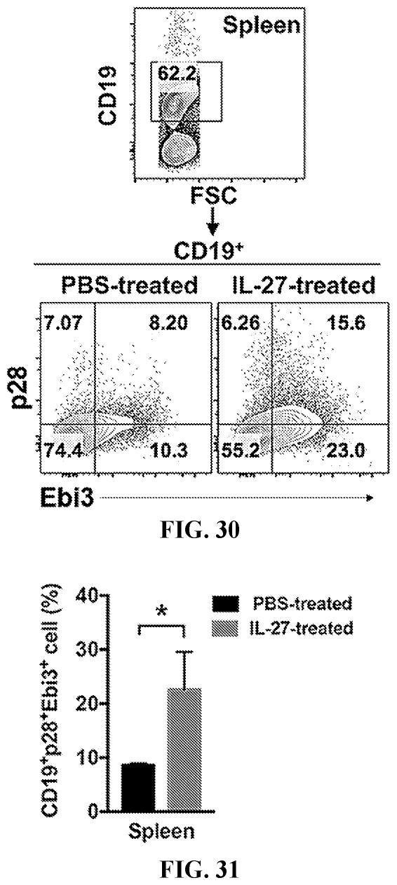

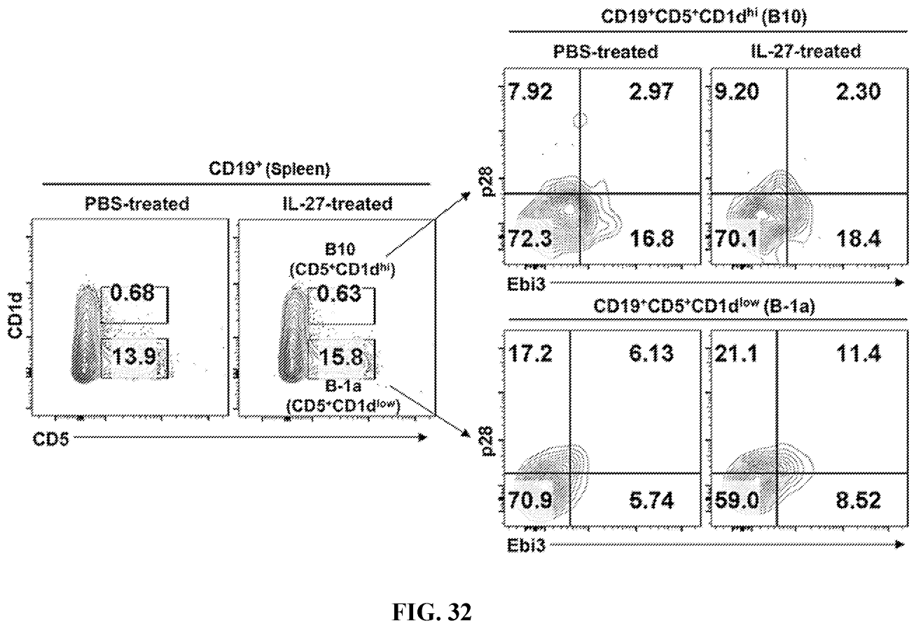

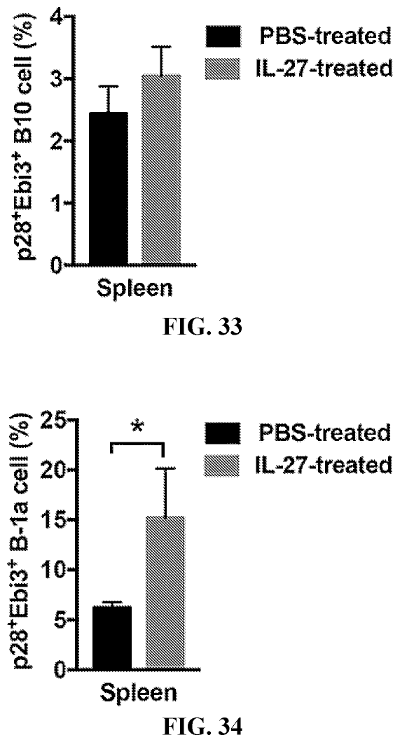

EAU is an animal model of human uveitis and is a predominantly T cell-mediated intraocular inflammatory disease induced by immunization with retinal proteins/peptides in CFA. The EAU model was used to investigate whether i27-Bregs contribute to regulating immunity during uveitis. EAU was induced in C57BL/6J mice by immunization with a peptide derived from interphotoreceptor-retinoid-binding protein (IRBP₆₅₁₋₆₇₀), and the mice were treated with PBS (control) or IL-27 concurrent with immunization. Fundus images of PBS-treated mice revealed characteristic features of uveitis including blurred optic disc margins, enlarged juxta-papillary area, moderate to severe retinal vasculitis, and cellular infiltrate (FIG. 18). In contrast, IL-27-treated mice were protected from EAU, exhibiting mild EAU with few cells and lower disease scores (FIG. 19). Histological analyses of PBS-treated eyes show inflammatory cells in vitreous, choroiditis, photoreceptor cell damage and retinal folds, but these hallmark features of uveitis were not observed in eyes of IL-27-treated mice (FIG. 20). Optical coherence tomography (OCT) shows substantial accumulation of inflammatory cells in vitreous and optic nerve head of PBS-treated, but not IL-27-treated, mice (FIG. 21), and visual impairment of control mice was not detectable by electroretinography (ERG) of IL-27-treated mice (FIGS. 22-25). Consistent with amelioration of EAU, an increase of IL-27 and a reduction of IL-17 in serum of IL-27-treated mice was detected (FIGS. 26-29). Other immune-suppressive cytokines including IL-10 and IL-35 were also elevated in the serum of IL-27-treated mice (FIGS. 26-29). Although intracellular cytokine analysis shows that about 8.2% B-cells in spleen of PBS-treated mice secreted IL-27, the percentage of i27-Bregs increased to more than 15% in IL-27-treated mice (FIGS. 30 and 31), indicating correlation between increase in i27-Bregs and amelioration of EAU. As B10 (CD19⁺CD5+CD1d^{hi}) and B-1a (CD19⁺CD5+CDlew) cells are CDS+and exhibit innate-like Breg functions, whether i27-Bregs induced during EAU derived from the B-1a or B10 pool was examined. Although PBS-treated mice contained modest levels of IL-27-producing B10 cells in their spleen (about 2.97%), the percentage of these i27-Bregs did not increase in IL-27-treated mice during EAU (FIGS. 32-34). In contrast, more than about 6% of B-1a cells in spleen of PBS-treated mice were i27-Bregs, which increased to greater than about 11% in IL-27-treated mice (FIGS. 32-34), suggesting that in vivo exposure to IL-27 further induced expansion of i27-producing B-1a cells during EAU.

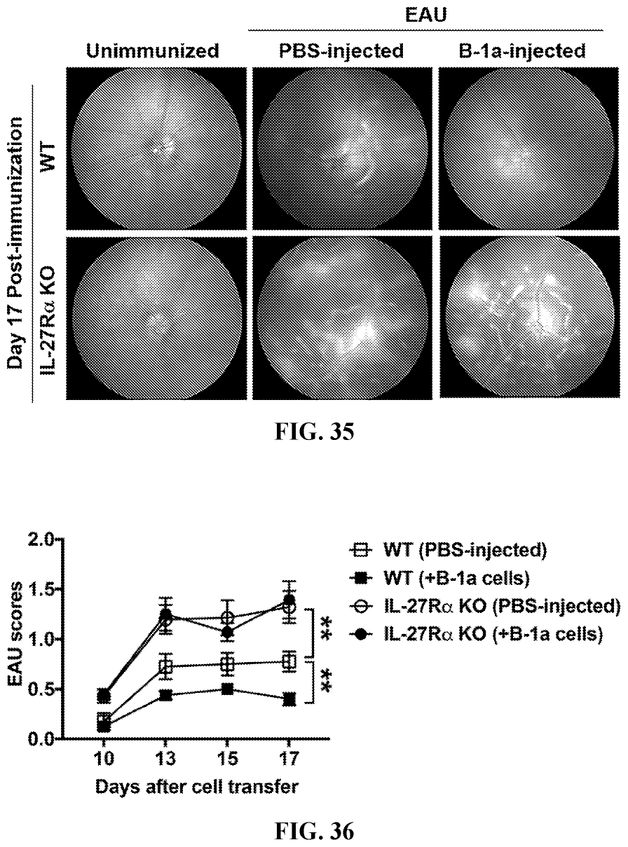

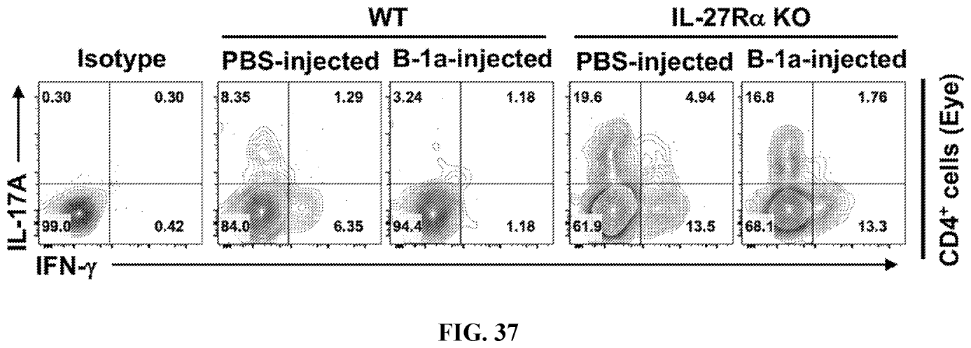

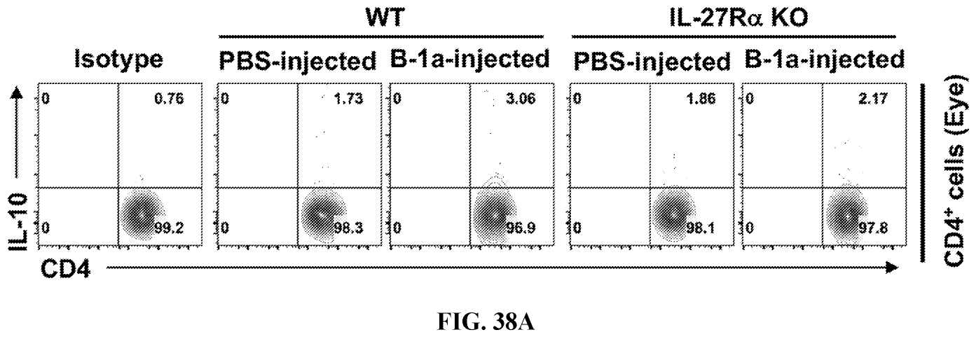

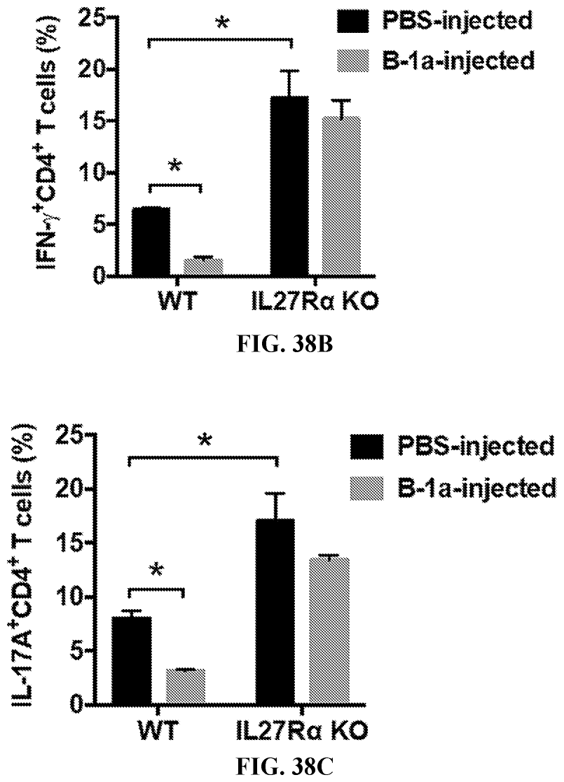

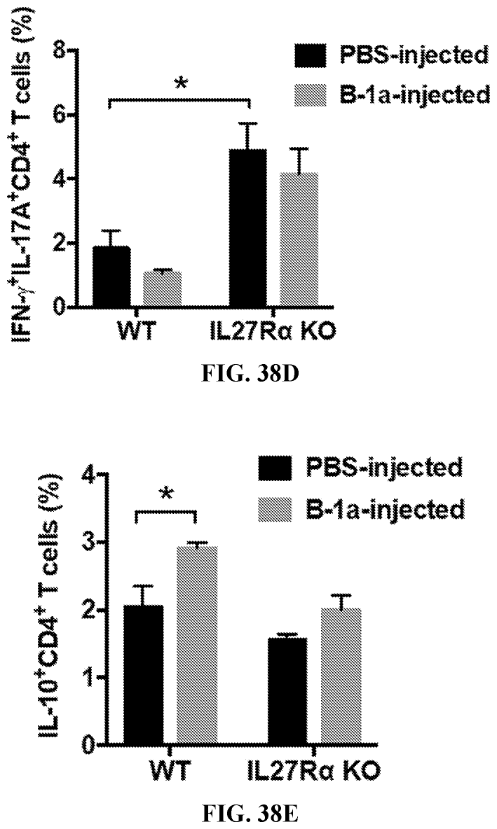

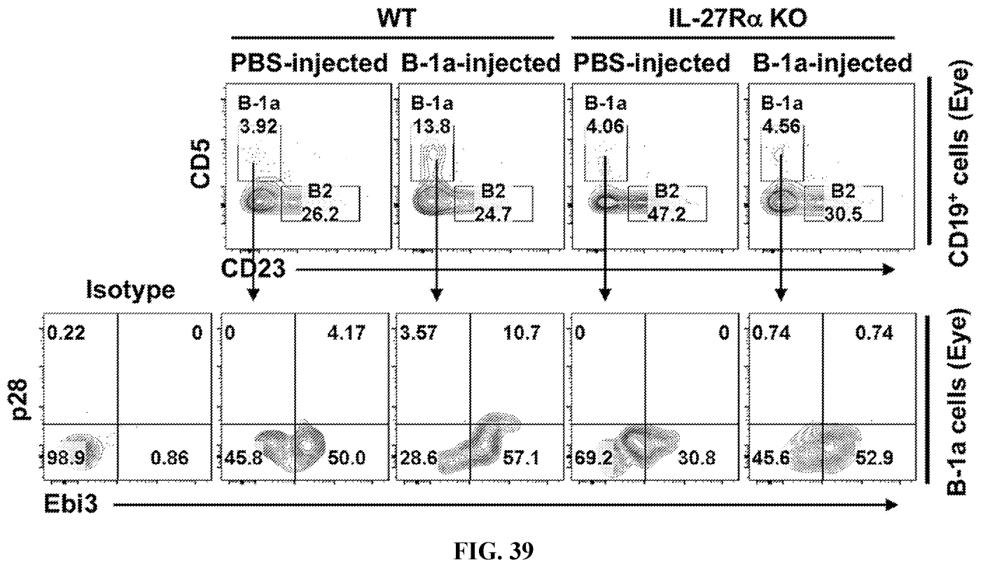

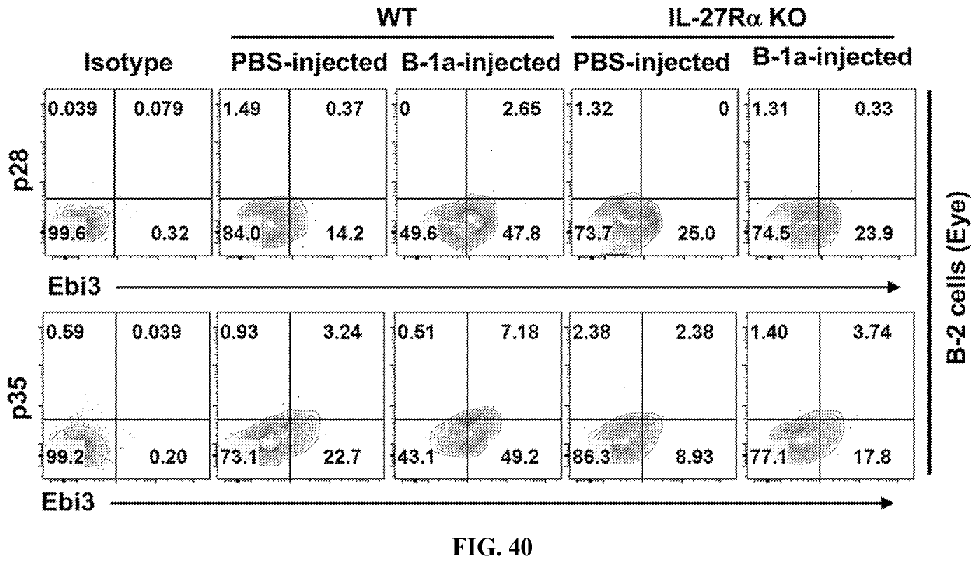

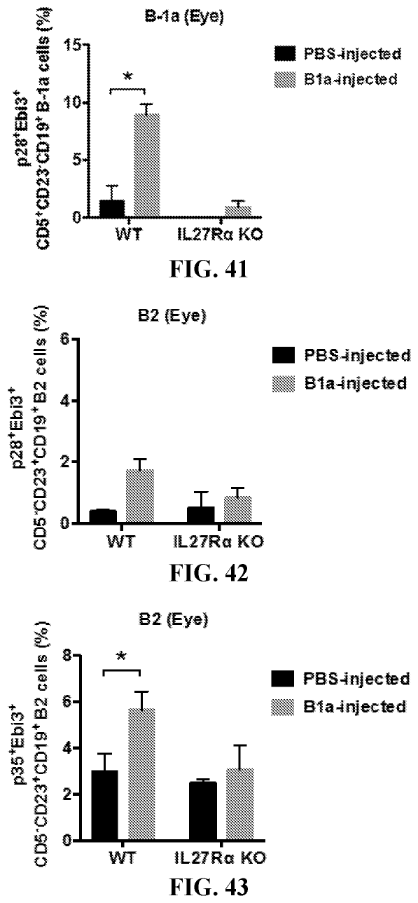

To investigate the potential therapeutic importance of i27-Bregs, peritoneal cavity B-1a cells (>80% i27-Bregs) were purified from WT donor CD45.2+mice with EAU, transferred 5×10⁵ cells/mouse to naive syngeneic WT or IL-27RαKO CD45.1⁺ mice, and then induced EAU 24 h after prophylactic administration of the B-1α cells. Fundus images on day-17 post-immunization showed severe uveitis in IL-27Rα-deficient mice (FIGS. 35 and 36) which correlated with an increase of Th1 and Th17 cells in the eye (FIGS. 37-38E). The PBS-injected group developed hallmark features of uveitis, albeit less severe as compared to IL-27RαKO mice. In contrast, mice given prophylactic B-1a cells developed only mild EAU (FIGS. 35 and 36) which correlated with a reduction of Thl/Th17 cells (FIG. 37) and concomitant increase of IL-27-producing B-1a cells (about 10.7%) in the eye (FIG. 39). This amelioration was not observed in IL-27Rα recipients, demonstrating that the amelioration was mediated by IL-27. Interestingly, B-1a therapy induced an about 2.2-fold expansion of IL-35-producing Breg cells (i35-Bregs) (FIG. 40).

Further, it was found that plasmacytoid dendritic cells induce expansion of i27-Breg cells as confirmed by flow cytometry following co-culture of activated IL-27-producing B-1a and plasmacytoid dendritic cells (1:1) (see FIGS. 149A-149B showing percentage of IL-27 secreting CD11b⁺B-1a cells).

### EXAMPLE 3

This example demonstrated that i27-Bregs in the brain and spinal cord suppress neuroinflammation and encephalomyelitis.

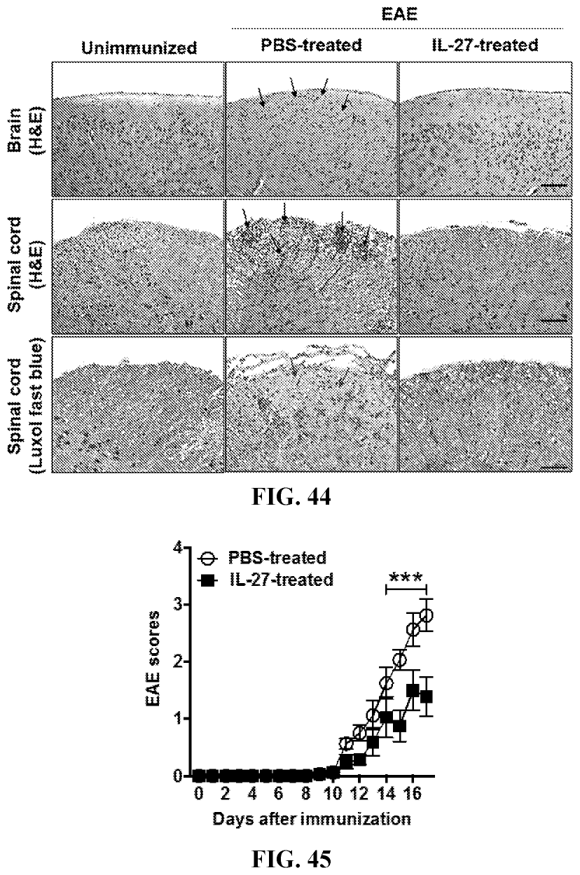

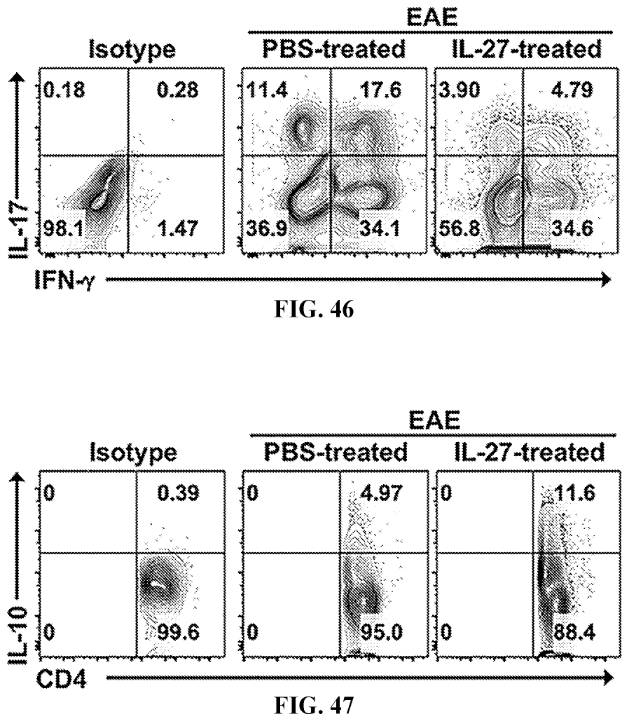

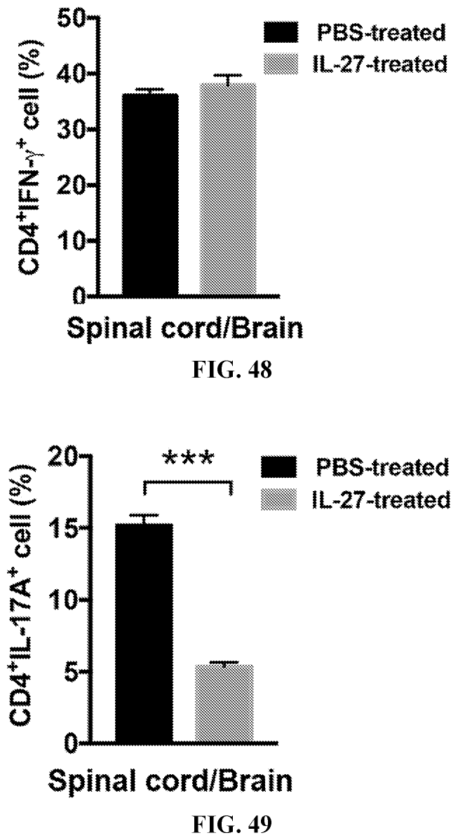

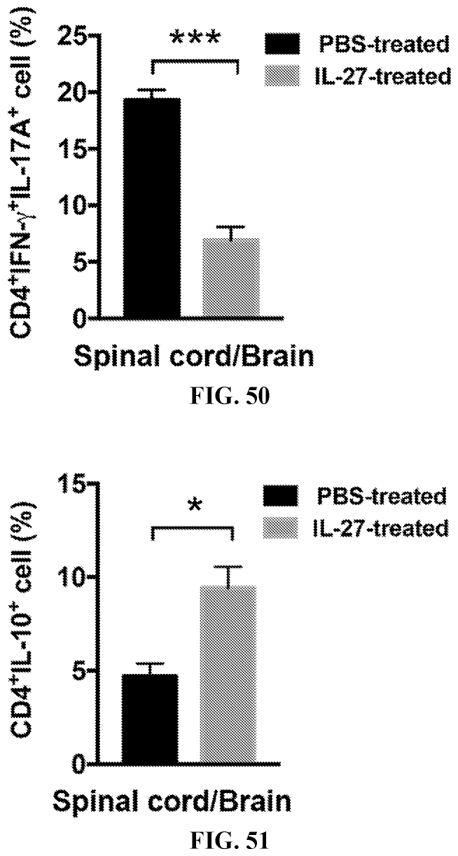

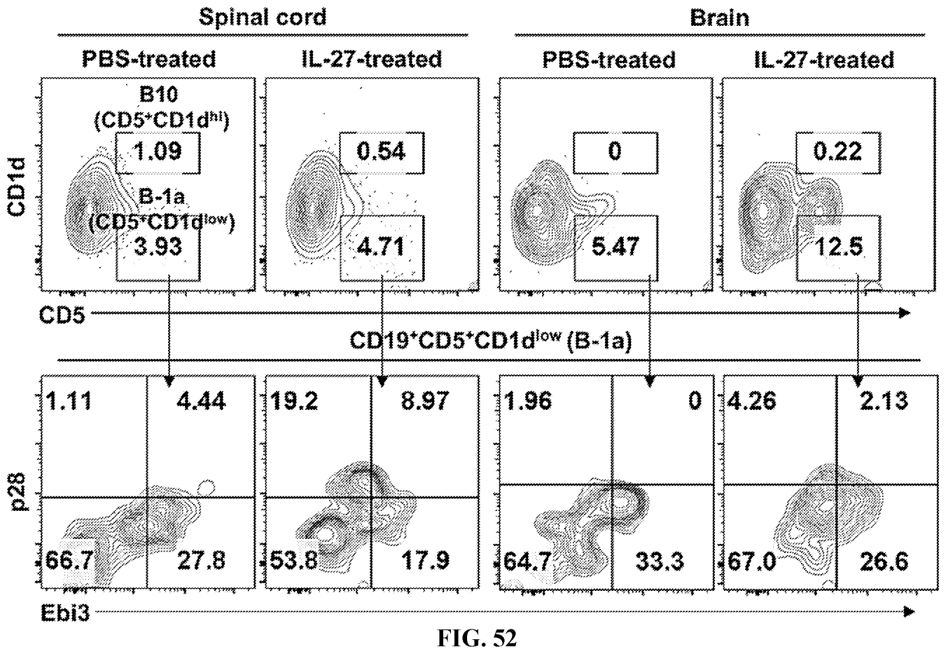

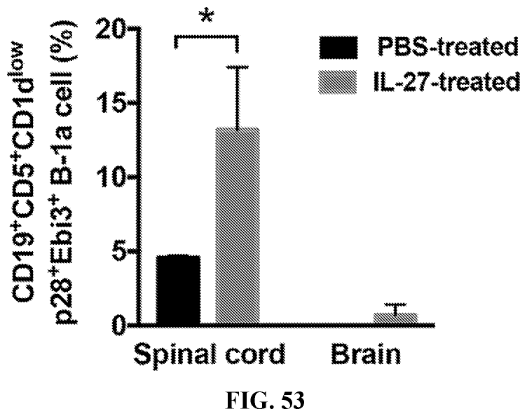

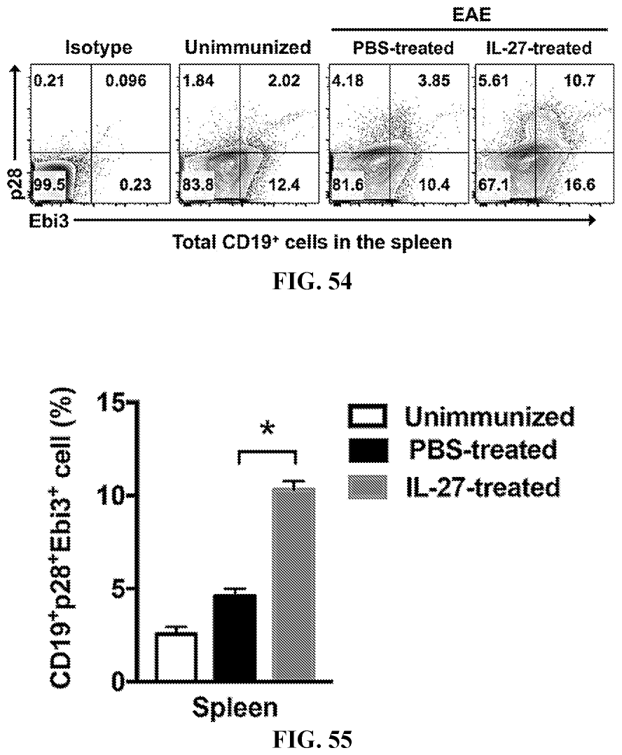

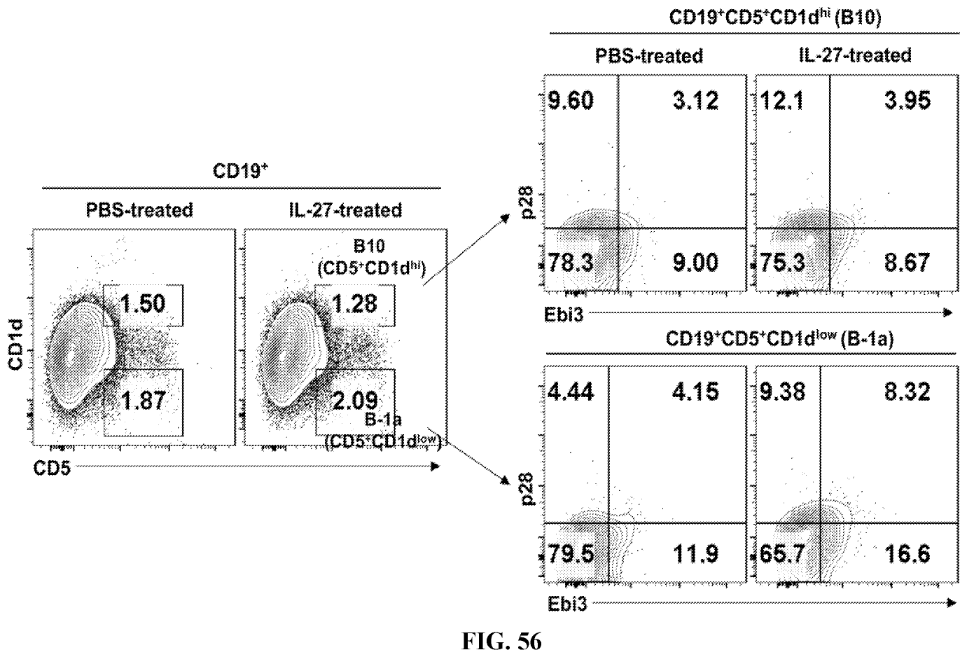

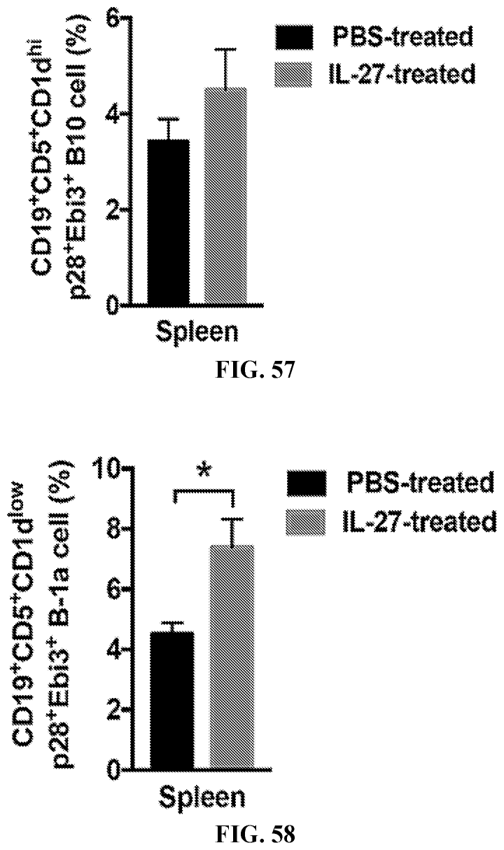

For these studies, the EAE model was used that shares essential immunopathogenic features with multiple sclerosis (MS) and exhibits progressive and relapsing-remitting forms of the human disease. EAE was induced by immunization of C57BL/6J mice with MOG35-55-peptide/CFA. Control PBS-treated mice developed EAE characterized by infiltration of inflammatory cells into the brain and spinal cord, flaccid tail, paraparesis, front/hind limb paralysis, and moribund state (FIG. 44). However, these hallmark features of EAE were much reduced in IL-27-treated mice, as indicated by histology and lower EAE clinical scores (FIG. 45). Disease attenuation correlated with significant reduction of the frequency of Th17 or IFN-γ/IL-17-expressing Th17 cells and increase of IL-10-expressing CD⁺ T cells in the brain and spinal cord of IL-27-treated mice (FIGS. 46-51). More importantly, i27-Breg cells in the spinal cord and brain of EAE mice (FIGS. 52 and 53) and significant levels of IL-27-producing B-1a cells in spinal cord (FIGS. 52 and 53) and spleen of IL-27-treated mice were detected (FIGS. 54-58).

The role of i27-Breg cells in suppressing EAE was further demonstrated in adoptive transfer studies using CD45.1⁺ and CD45.2⁺ congenic mouse strains. CD45.2+ mice were immunized with MOG₃₅₋₅₅-peptide/CFA and treated with PBS or IL-27. Encephalitogenic cells were harvested from the spleen and LN 21 days after immunization, and 10×10⁶ cells from PBS-treated or IL-27-treated CD45.2⁺ mice were adoptively transferred to unimmunized CD45.1⁺ mice and evaluated for EAE development and severity. Transfer of cells from PBS-treated mice induced disease with characteristic features of EAE, while CD45.1⁺ mice that received CD45.2⁺ cells from IL-27-treated mice developed mild

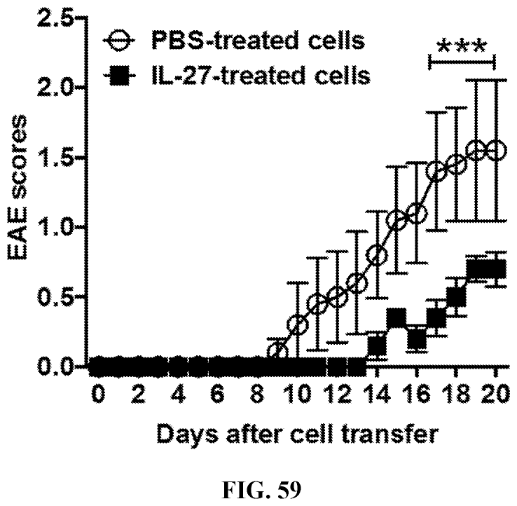

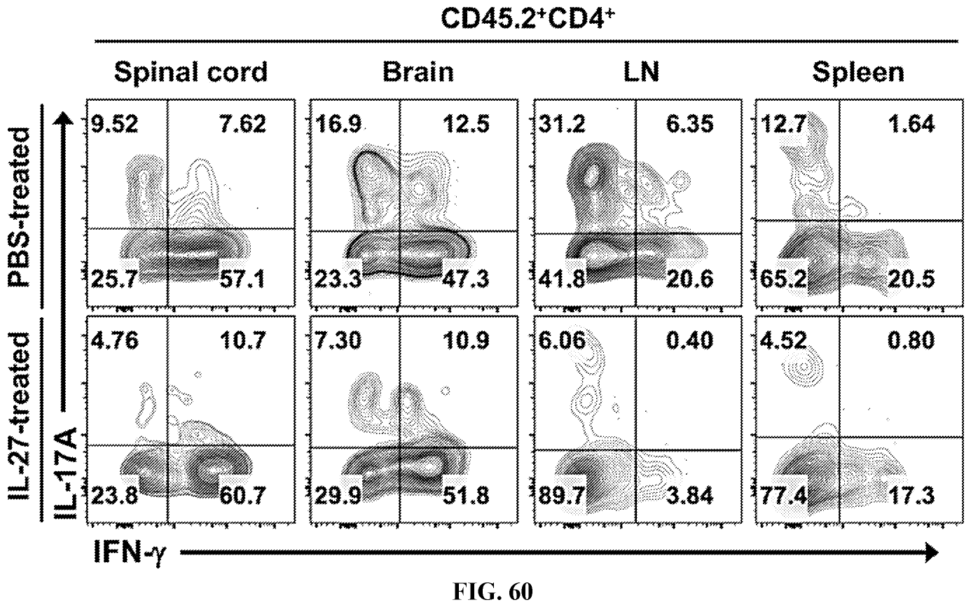

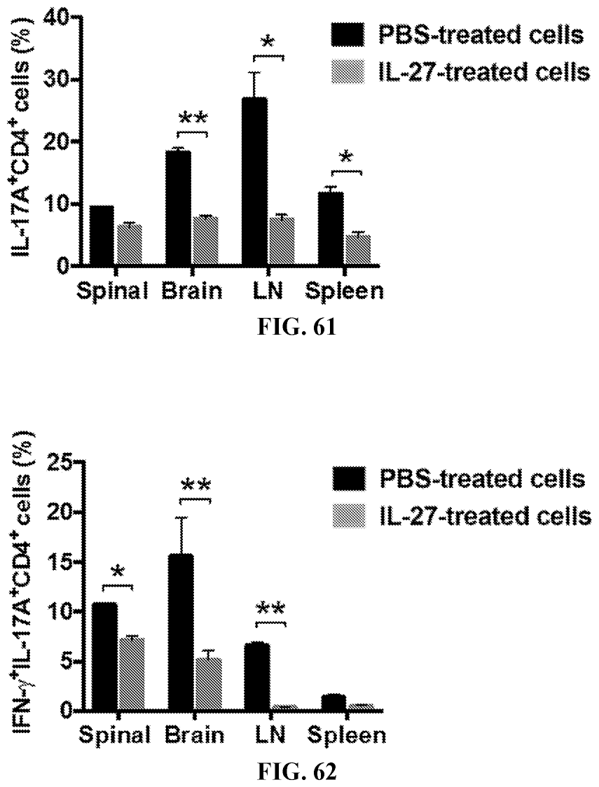

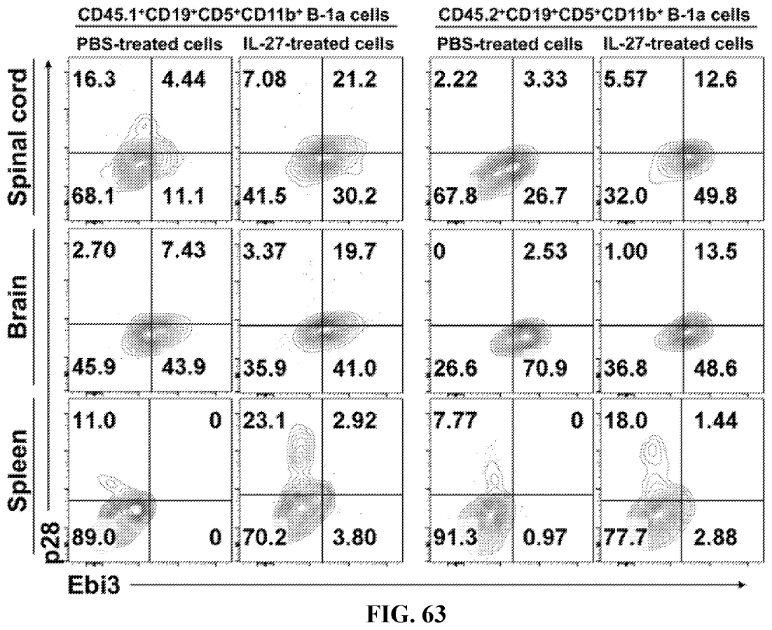

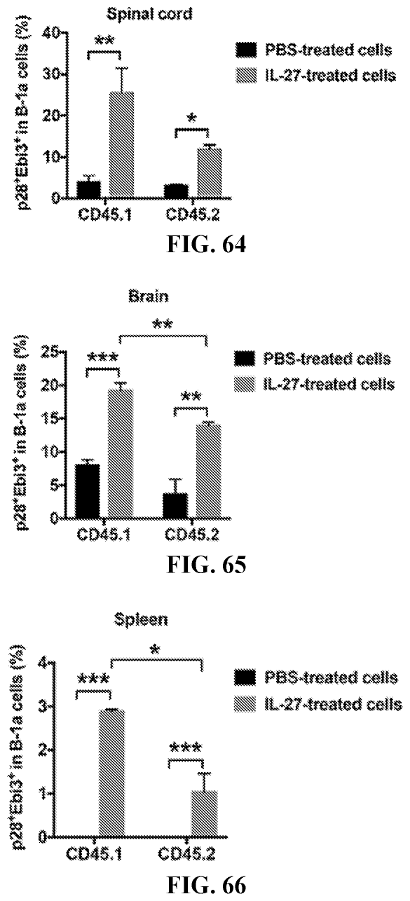

EAE with delayed onset (FIG. 59). Reduced EAE in the recipient mice derived in part from suppression of Th17 responses (FIG. 60) and concomitant expansion of IL-27-producing B-1a cells (FIGS. 63-66). It is notable that levels of CD45.2⁺ IL-27-producing B-1a cells increased insignificantly in spinal cord, brain, and spleen of recipient IL-27-treated mice (FIGS. 63-66), indicating that the transferred CD45.2⁺ i27-Bregs cells might have proliferated in vivo. Most remarkable is that recruitment of CD45.2⁺ Bregs into CNS tissues promoted expansion of endogenous CD45.1⁺ Bregs (FIGS. 63-66). Expansion of transferred i27-Bregs and endogenous CD45.1⁺ Bregs in spinal cord and brain would therefore sustain prolonged production of IL-27 in host tissues. Thus, i27-Breg therapy could provide a therapeutic advantage over administering IL-27, which is rapidly cleared in vivo.

EXAMPLE 4

This example demonstrated that innate IL-27-producing B-1a cells suppress EAE and EAU in antigen-independent manner.

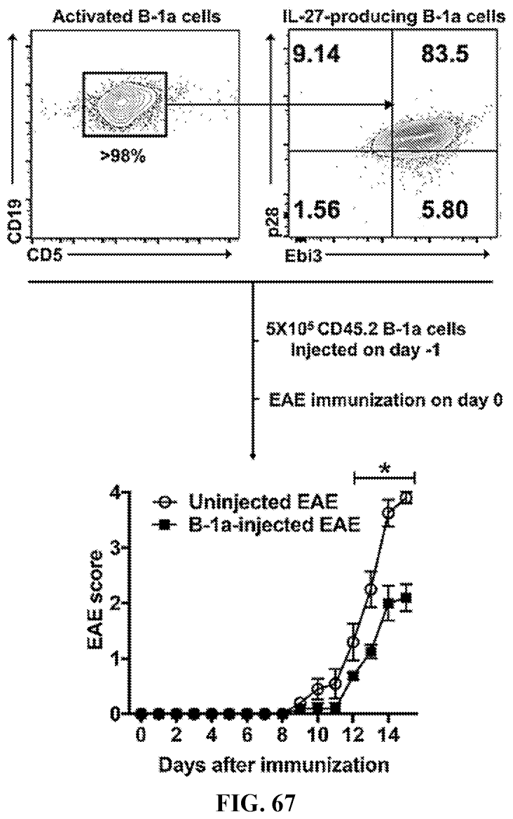

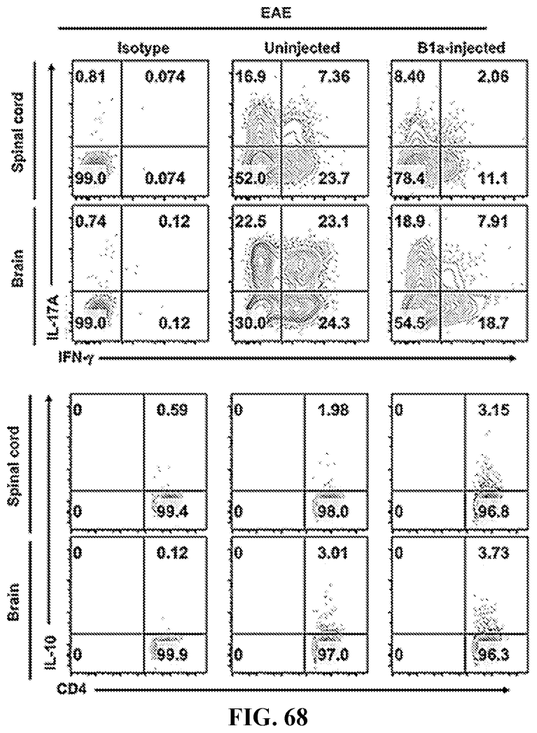

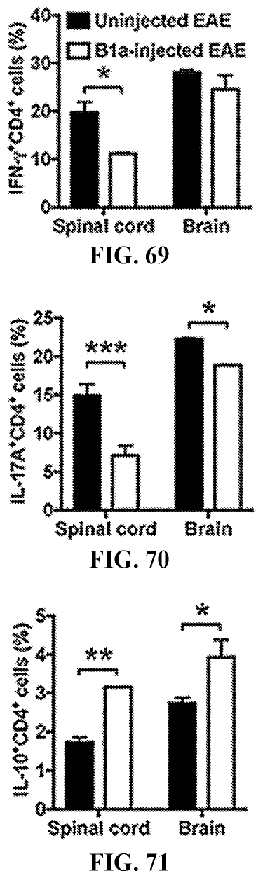

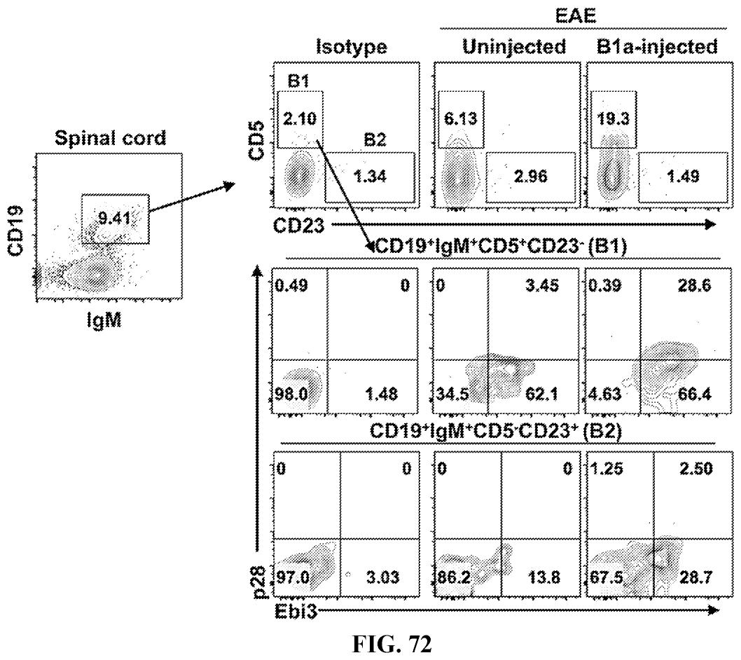

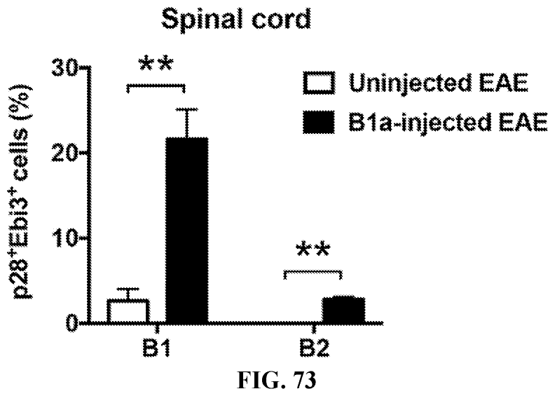

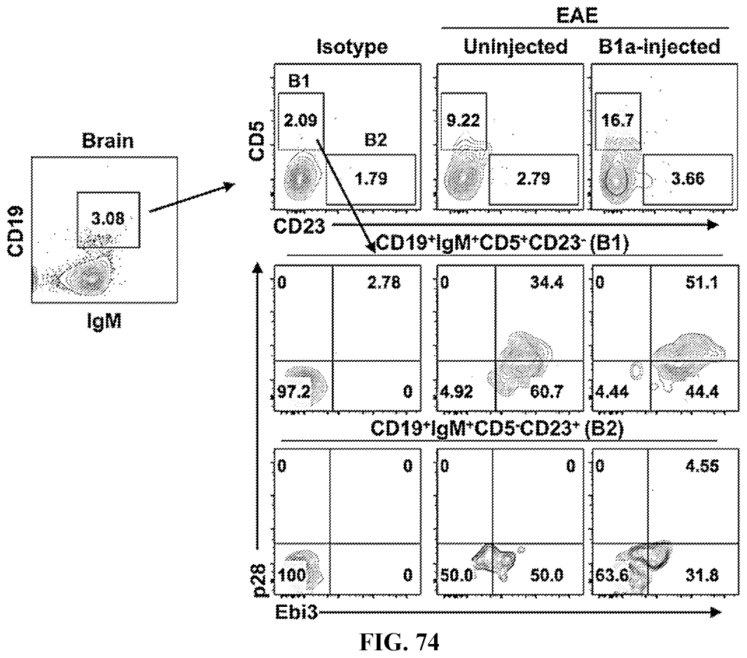

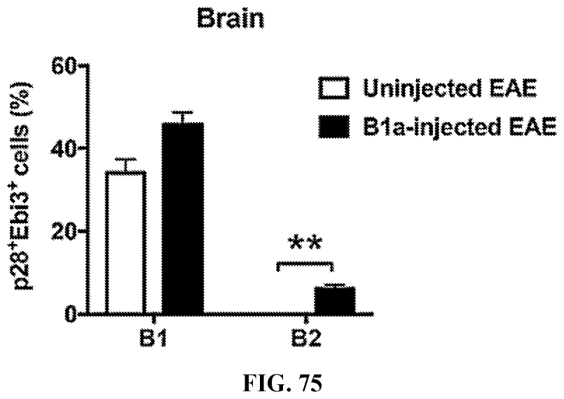

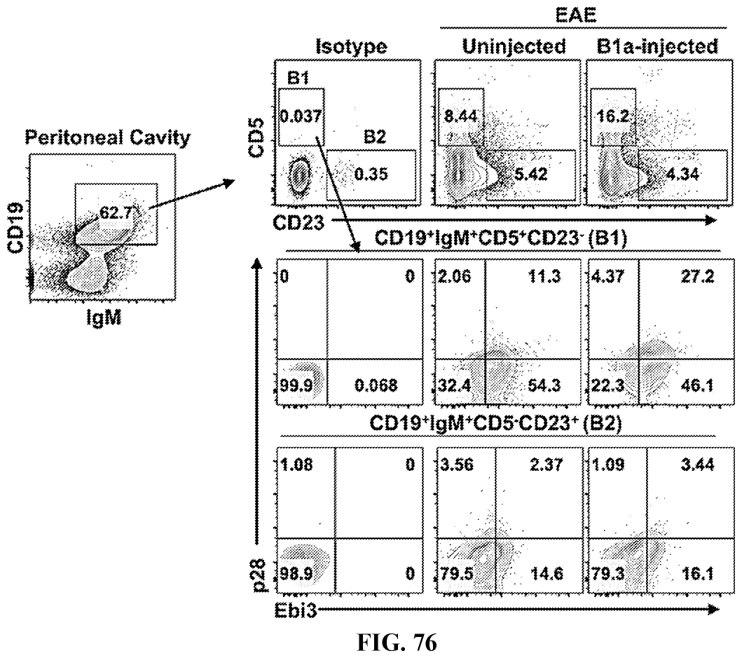

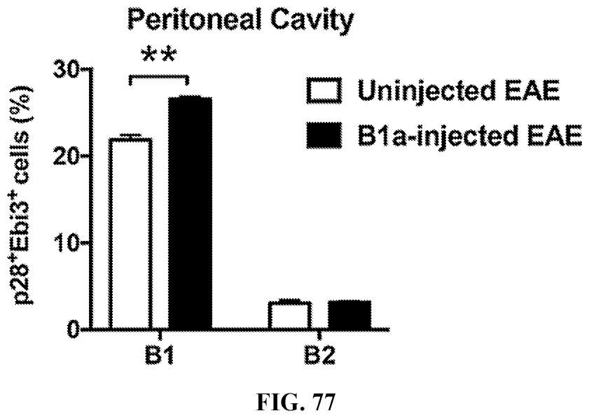

Bregs are mostly antigen-specific and effective in suppressing diseases mediated by lymphocytes that recognize the same cognate autoantigen. Thus, it was investigated whether IL-27-producing B-1a cells induced by an irrelevant stimulus like LPS could suppress encephalitogenic lymphocytes that mediate EAE. CD45.2⁺ C57BL/6J mice were injected with LPS, and after 2 days purified B-1a cells were obtained from the peritoneal cavity (>80% B-1a i27-Bregs). The i27-Bregs then were transferred into naïve CD45.1⁺ congenic mice. EAE was induced in recipient CD45.1⁺ mice by immunization with MOG35(n=7) 24 h after adoptive transfer. Transfer of the ex-vivo generated B-1a i27-Bregs (5 x10⁵ cells/mouse) suppressed EAE (FIG. 67), and disease amelioration was correlated with a reduction of IL-17-single positive and IL-17/IFN-γ-double positive T-cells and an increase in IL-10-producing regulatory CD4⁺ T-cells in the brain and spinal cord (FIGS. 68-71). Suppression of EAE also correlated with an increase of i27-Breg cells in the spinal cord (FIGS. 72 and 73), brain (FIGS. 74 and 75), and the peritoneal cavity (FIGS. 76 and 77), and the majority of the i27-Breg cells were observed to be B-1a cells. Similar results were obtained in the EAU model. Thus, in line with its developmental origin, suppression of CNS autoimmune diseases by innate i27-Breg cells does not require prior activation by the autoantigen that elicited EAE or EAU. This result contrasts to B2 Breg therapy that mediates Ag-specific immune suppression and suggests that transfer of autologous innate i27-Breg cells can be exploited as a treatment for a wider array of autoimmune diseases.

### EXAMPLE 5

This example demonstrated that cross talk exists between IL-27-producing B-1a and lymphoid or myeloid cells in the CNS.

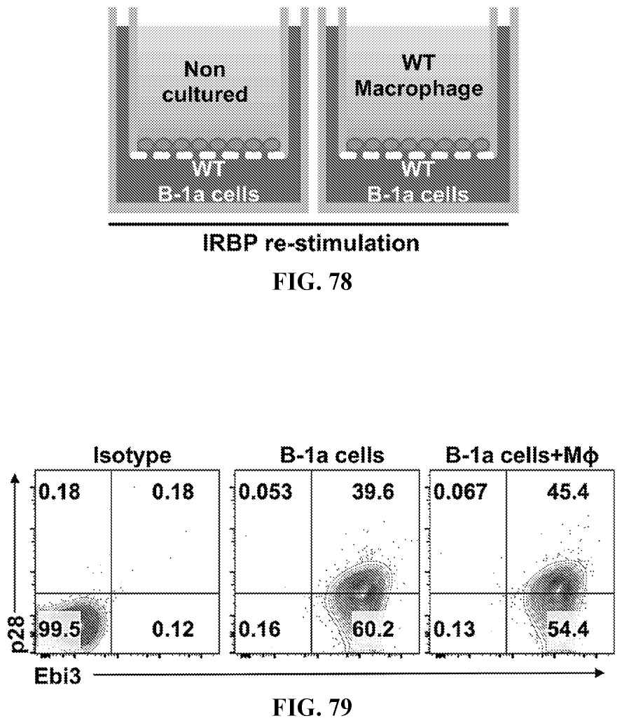

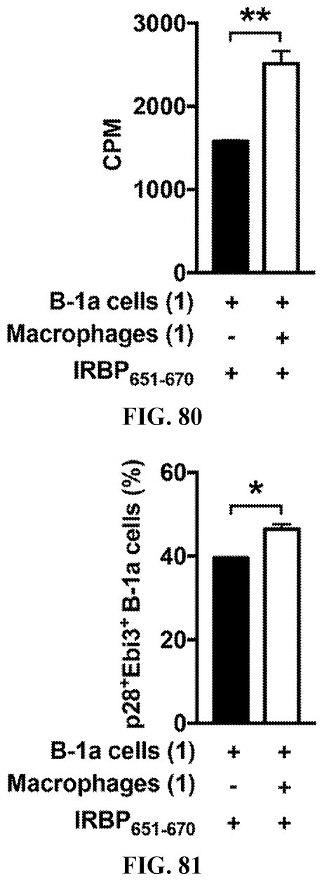

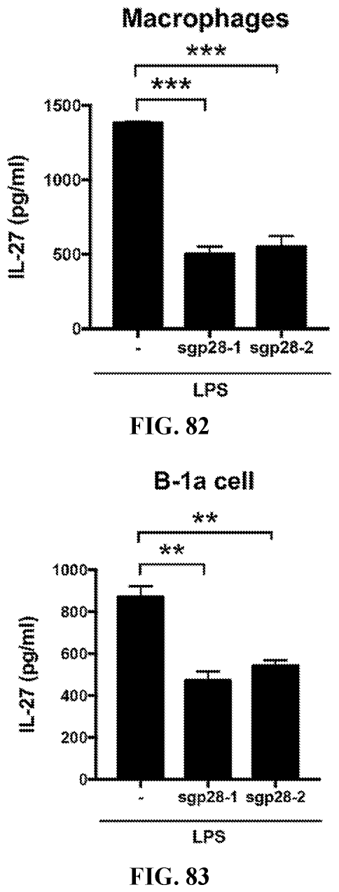

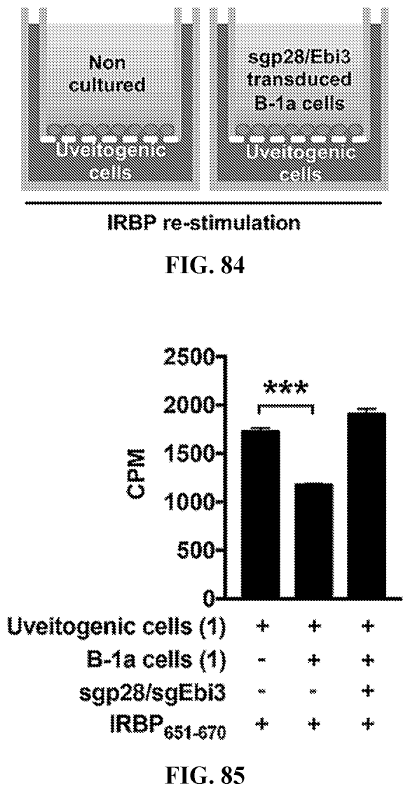

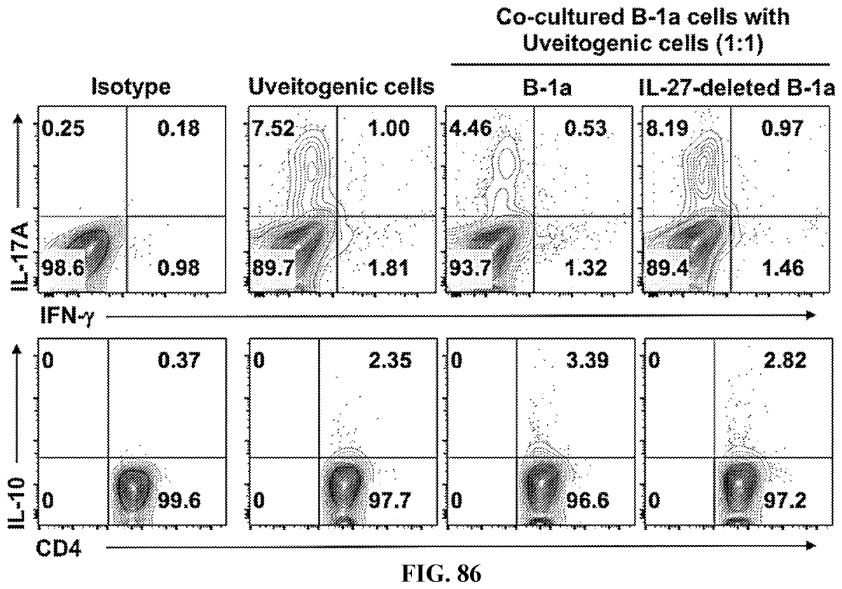

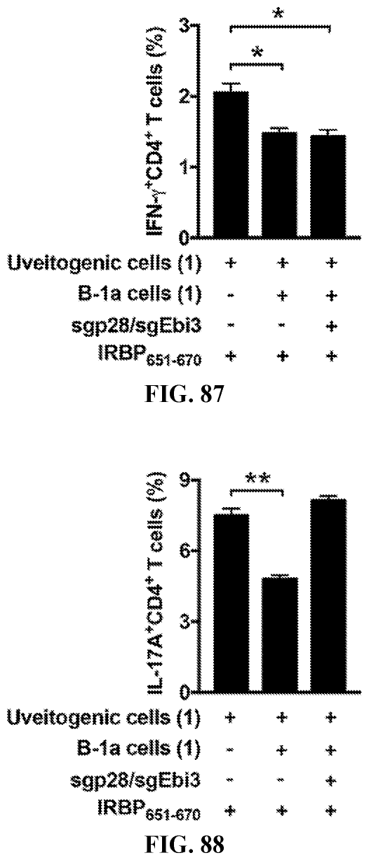

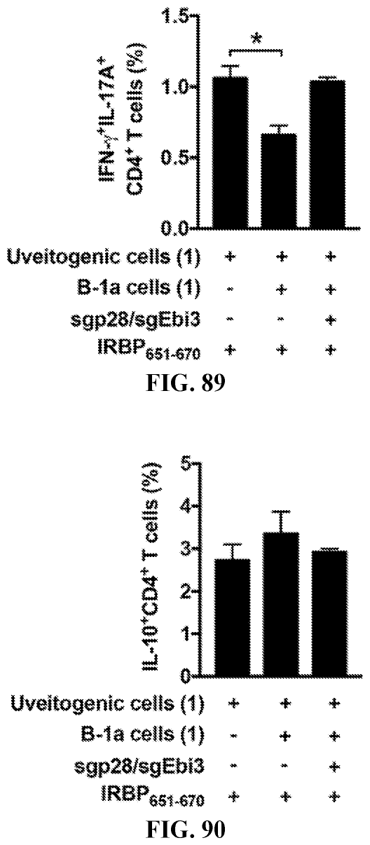

This study examined whether i27-Bregs that enter the CNS during EAE or EAU might be a source of IL-27 that contributes to immune-suppressive environment of the CNS. B-1a cells and macrophages from IRBP-immunized wild type were sorted, and it was found that co-culture of the cells for 3 days in a trans-well system significantly increased of IL-27-producing B-1a cells (FIGS. 78-81), suggesting that soluble mediator(s) produced by myeloid cells might increase IL-27 levels in the retina during uveitis by promoting the expansion of i27-Breg cells. The data further shows that, like B-1a cells, macrophages respond to inflammatory stimulus by producing IL-27 (FIGS. 82 and 83). However, infection of either cell type with Lentivirus expressing sgp28/sgpEbi3 guide RNA, that targets p28 and ebi3 expression, suppressed capacity of the macrophages or B-1a cells to produce IL-27 (FIGS. 82 and 83), suggesting that i27-Bregs might synergize with myeloid cells to increase IL-27 levels in the CNS during inflammation. The potential cross-talk between i27-Bregs and lymphocytes that mediate CNS autoimmune diseases was also examined. Co-culture with B-1a cells suppressed the proliferation (FIGS. 84-90) of uveitogenic T-cells in the spleen and lymph nodes of EAU mice. The capacity to suppress Th17-induced inflammatory responses was curtailed if the B-1a cells were defective in IL-27 expression (FIGS. 86-90). These results suggest that B-1a cells that enter the retina can suppress Th17 cells during EAU through paracrine effects of the IL-27 they secrete.

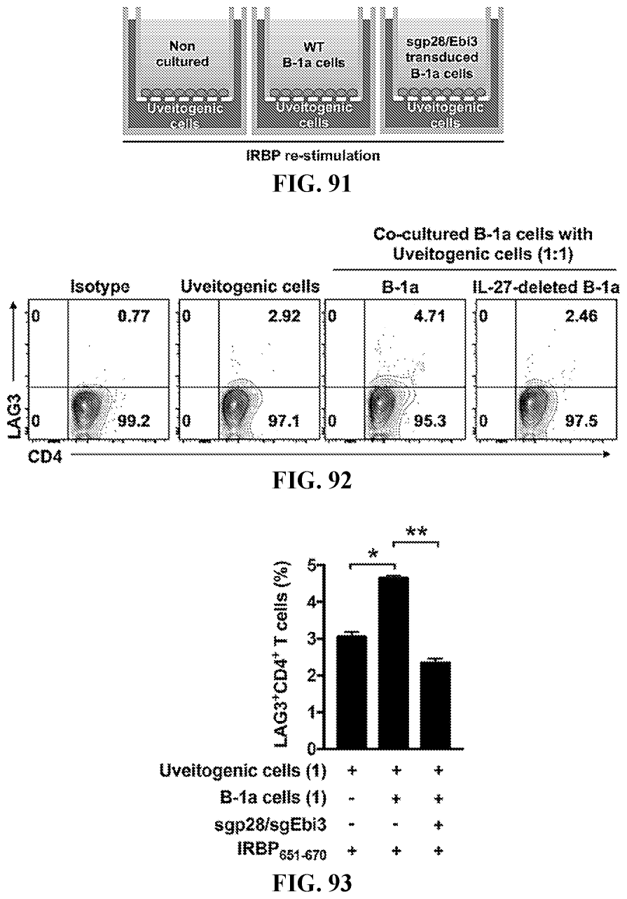

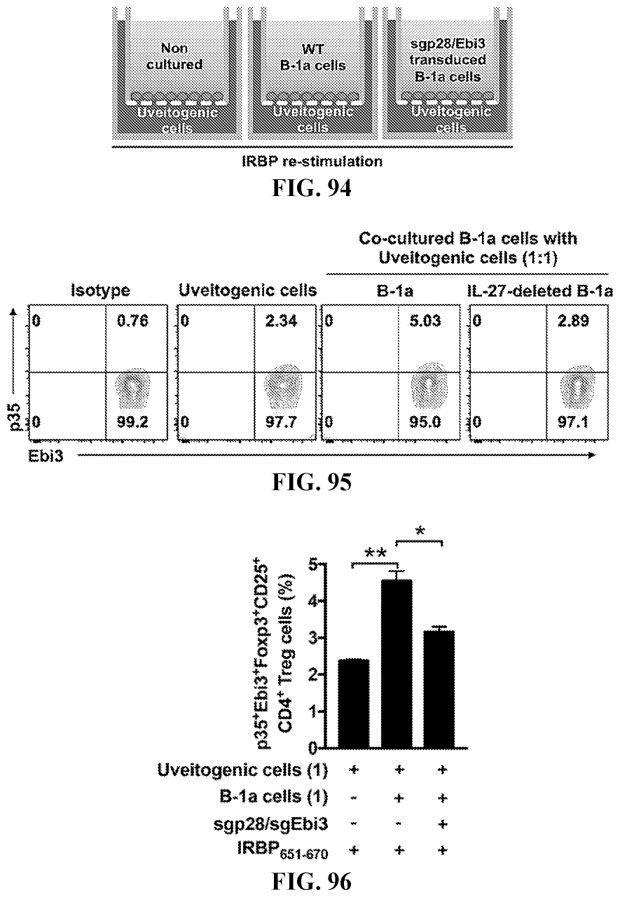

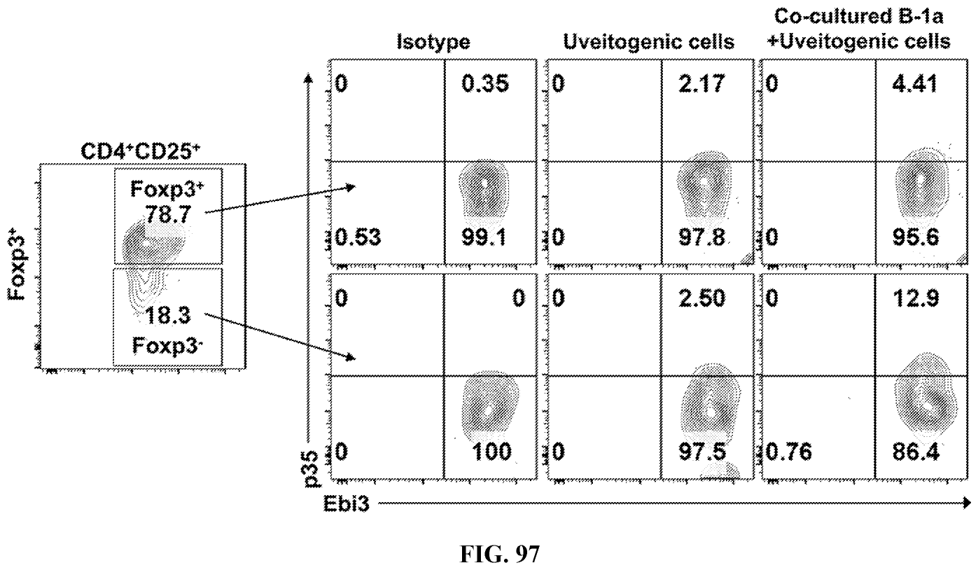

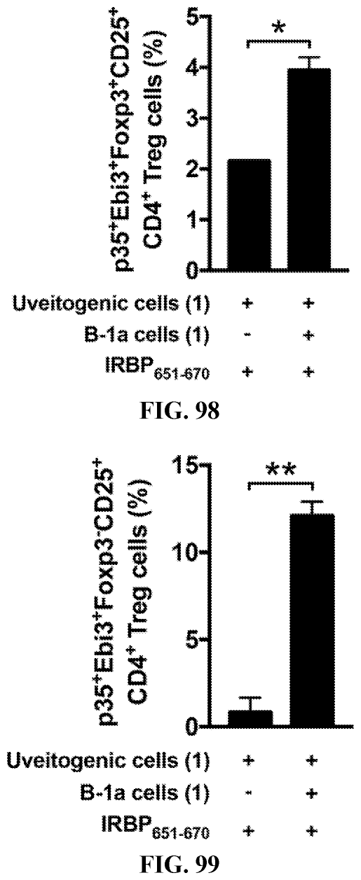

The data show that co-culture of B-1a cells and uveitogenic T-cells induced the expansion of CD4⁺ T-cells expressing the inhibitory receptor, LAG-3 (LAG-3+ CD4+ T-cells) (FIGS. 91-93), and i35-Bregs (FIGS. 94-96) in an IL-27-dependent manner. Interestingly, the majority of the IL-35-producing cells induced by IL-27 were Foxp3-negative (FIGS. 97-99). These results suggest that i27-Bregs can suppress intraocular inflammation, at least in part, by inducing effector T-cells to acquire regulatory phenotype and functions.

### EXAMPLE 6

This example demonstrated that IL-27 regulates B1 and B2 cells differently.

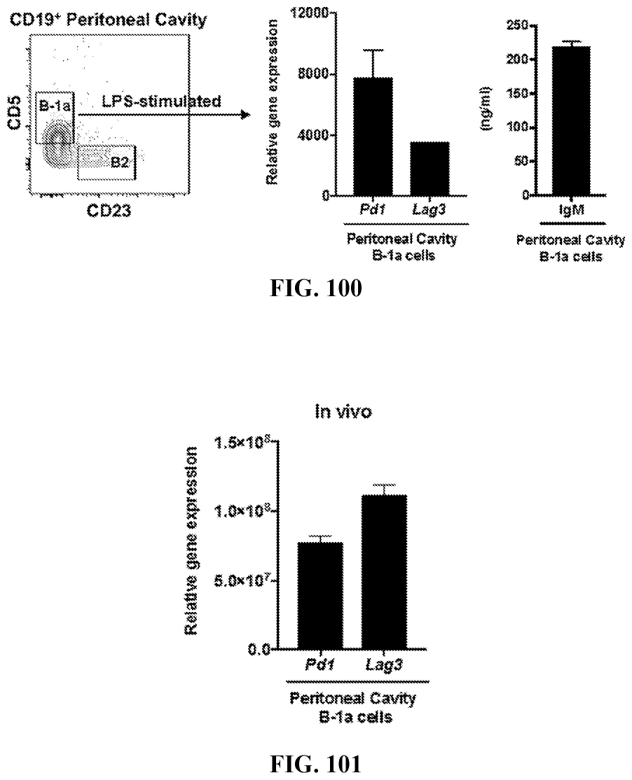

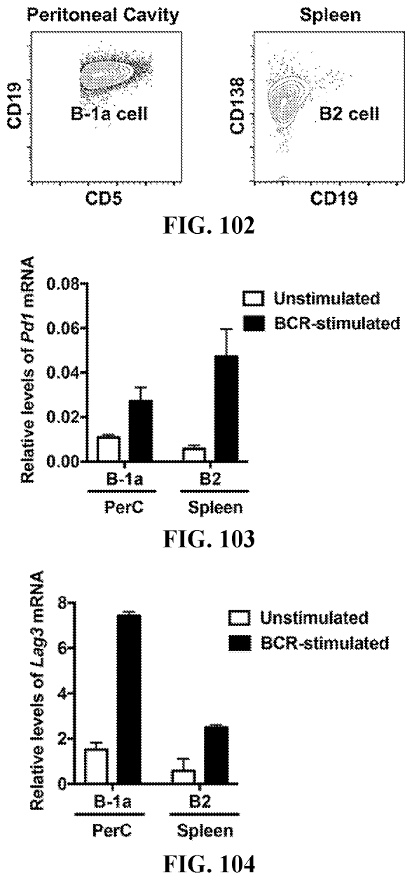

This study examines whether the B-1a cells that suppress EAU and EAE through production of IL-27 also suppress inflammation by expressing inhibitory molecules. B-1a cells were isolated from mouse peritoneal cavity by sorting. The cells were then stimulated for 48 h with LPS and demonstrated that they were B-1a cells by their capacity to produce IgM (FIG. 100). Analysis of cDNA prepared from the cells by qPCR revealed that B-1a cells can indeed express Lag3 and Pdl (FIG. 100). The in vivo LPS model was used to investigate whether innate B-1a cells also express these inhibitory receptors in response to inflammatory challenge, as occurs during EAE, EAU, or sepsis. C57BL/6J mice were injected (i.v) with LPS, and purified B-1a cells were isolated from the peritoneal cavity by magnetic bead sorting. The results of the qPCR analysis of cDNAs derived from the cells 48 h after LPS administration confirmed that transcription of Lag3 and Pd1 was upregulated by B-1a cells in the peritoneal cavity (FIG. 101). As LAG-3⁺CD138⁺ natural regulatory plasma cells develop via an antigen-specific mechanism, B-1a and B2 cells sorted from the mouse peritoneal cavity or spleen and cells stimulated with anti-IgM/anti-CD40 showed that both B-1a and plasma cells upregulate transcription of Lag3 and Pdl in response to BCR signaling, as shown by qPCR analysis (FIGS. 102-104). Taken together, these observations suggest that in response to stimulation by a pathogen (e.g., a TLR agonist) or autoantigen, B-1a cells can acquire capacity to express inhibitory molecules that enhance their immune-regulatory activities.

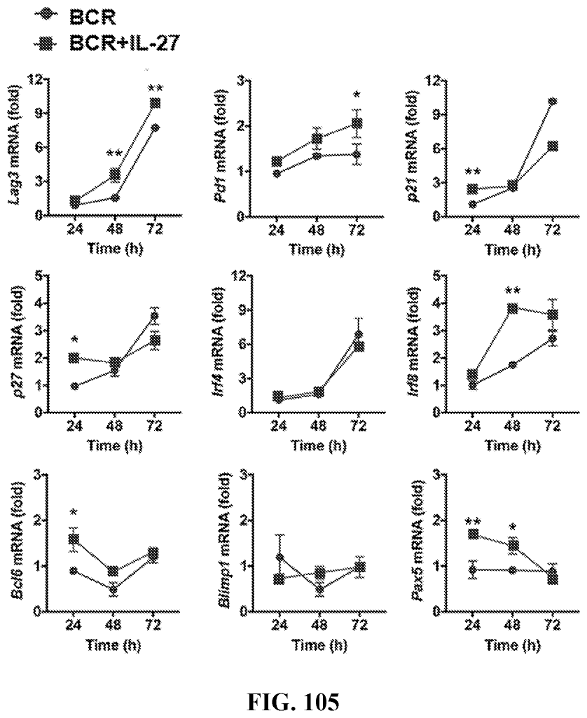

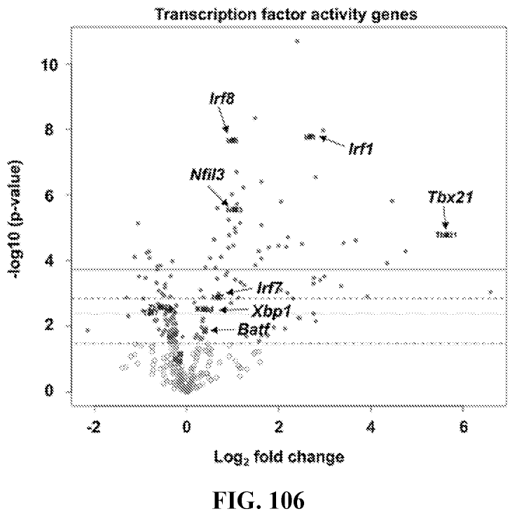

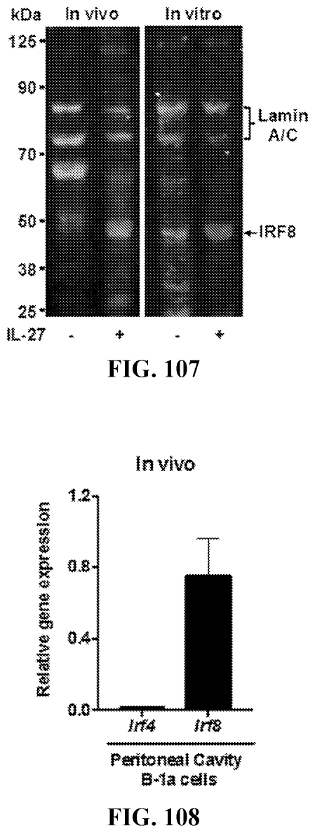

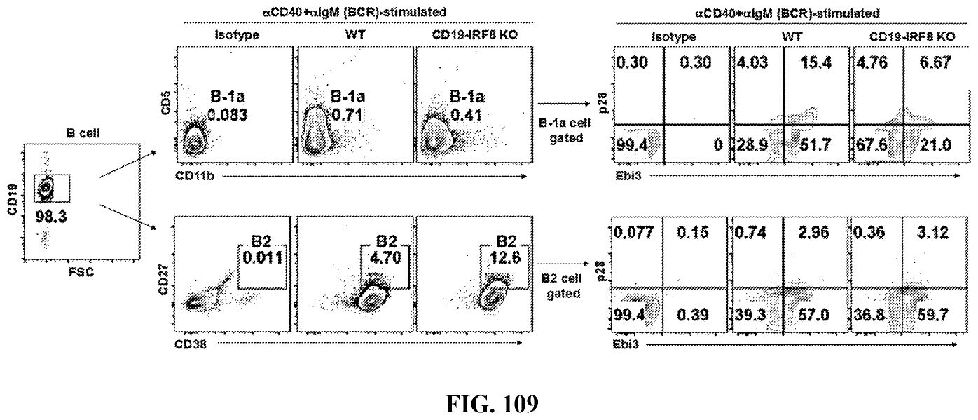

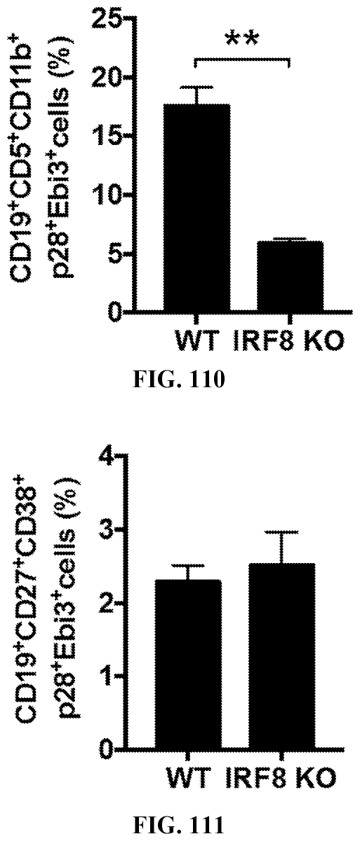

A previous report indicated that IL-35-producing B-cells are exclusively B2 CD138⁺ plasma cells (Shen et al., Nature, 507: 366-370 (2014)). However, this study shows that IL-27-producing B-cells derive from the B1 compartment. To understand mechanisms that skew activated B-cells toward the i27-Breg developmental program, the transcriptome of activated CD19⁺ B-cells stimulated with IL-27 was profiled. qPCR (FIG. 105) and NanoString (FIG. 106) RNA analyses identified several genes differentially activated by IL-27 (Irf8, Irfl, Tbx21, Nfil3, Irf7, Xbp1, and Batf), some of which are known to regulate critical pathways in B-cells. Of particular interest was the differential upregulation of IRF-8 and IRF-4, as these transcription factors are implicated in B-cell development and effector functions. In view of reports that mutual antagonism between IRF-4 and IRF-8 regulates B-cell development, with an increase of IL-4 favoring plasma cell development (see, for example, Xu et al., Nat. Immunol., 16: 1274-1281 (2015)), preferential upregulation of Irf8 by B-1a cells might drive i27-Breg developmental program. As IL-27 induces expansion of i27-Bregs, whether IRF-8 activates transcription of Il127a that codes for the IL-27p28 subunit protein was investigated. It is therefore of note that IRF-8 and IRF-4 activate transcription through hetero-dimerization with ETS/PU-1 or BATF families of transcription factors, resulting in their recruitment to ETS-IRF (EICEs) or AP1-IRF (AICEs) composite elements of immune-regulatory genes. EMSA and Super-shift analyses using validated AICE sites relevant to expression of 127a or Ctla4 show IL-27 induces formation of AICE complexes in activated B-cells under in-vivo or in-vitro conditions. In addition, both IRF-4 and IRF-8 were recruited to AICE of Ctla4, while IRF-8 but not IRF4 was recruited to AICE of1127a, thereby suggesting that IRF-8 promotes expression of IL-27 in B-cells. Western blot analysis confirmed that IL-27 upregulates IRF-8 in B-cells (FIG. 107), and RNA analysis showed up-regulated transcription of Irf8 by B-1a cells isolated from mice injected with LPS (FIG. 108), thereby suggesting an IRF-8/IL-27 axis that might orchestrate a reciprocal autoregulatory loop that promotes expression of IRF-8 and IL-27 in B-1a cells. A significant reduction of IL-27-producing B-1a cells of CD19-IRF8KO mice (FIGS. 108-111) was also observed, which further underscores the role of IRF-8 in promoting expansion of i27-Breg cells. These results suggest that preferential activation of the IRF-8/IL-27 axis in the B1 compartment may skew activated B-1a cells toward the i27-Breg developmental program.

### EXAMPLE 7

This example demonstrated that i27-Breg cells exist in humans and can be expanded in response to inflammatory stimuli.

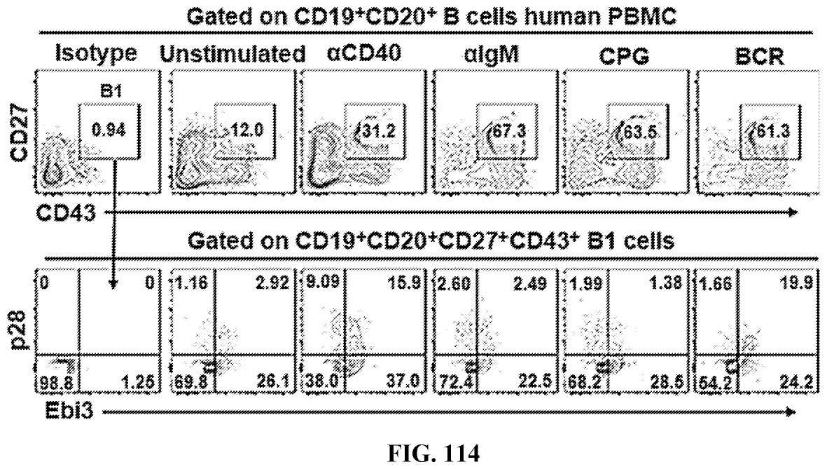

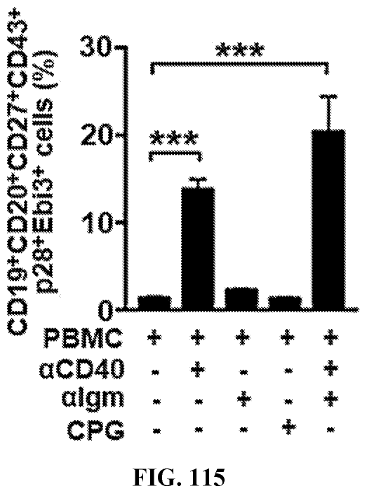

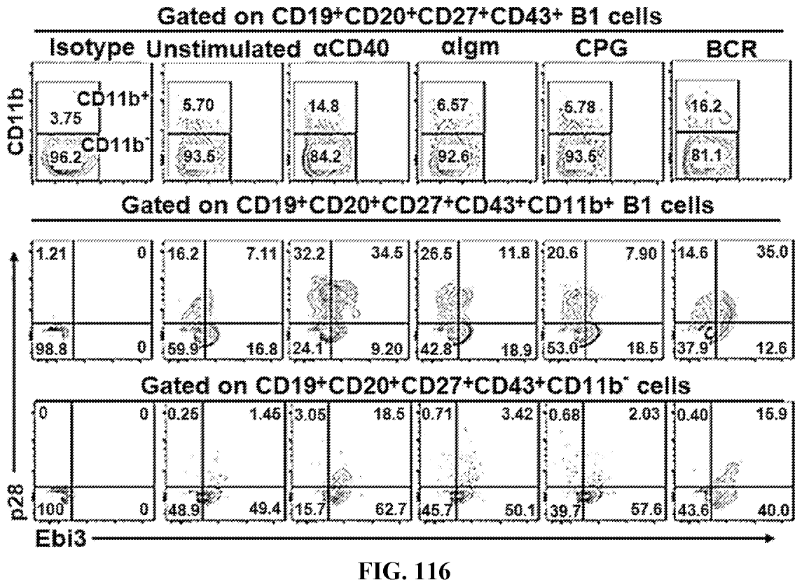

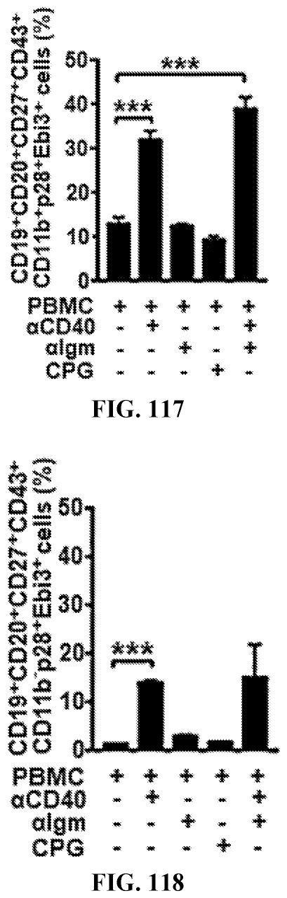

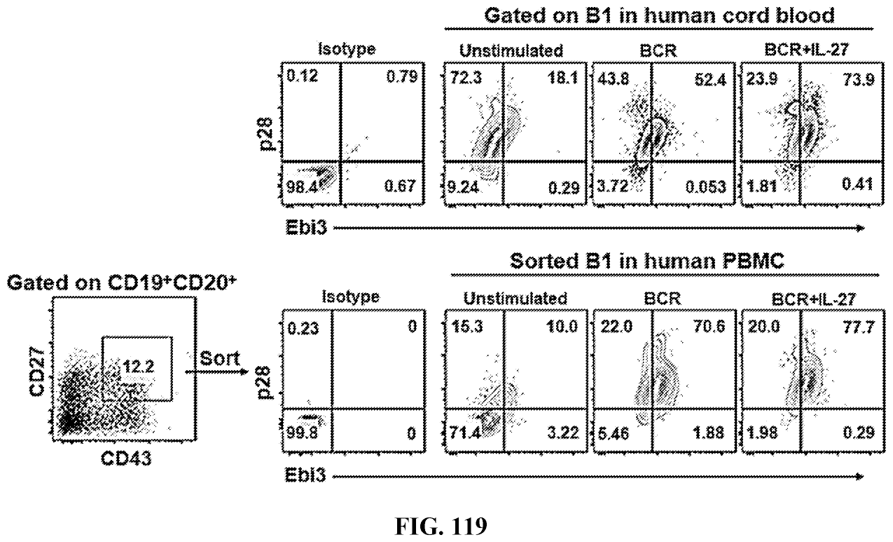

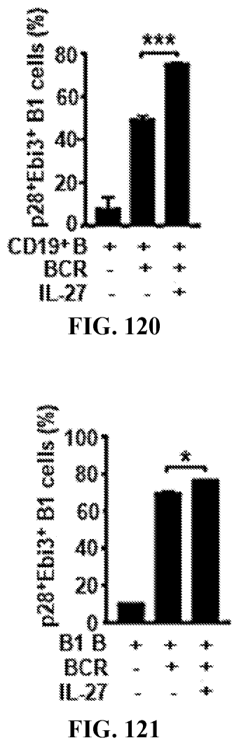

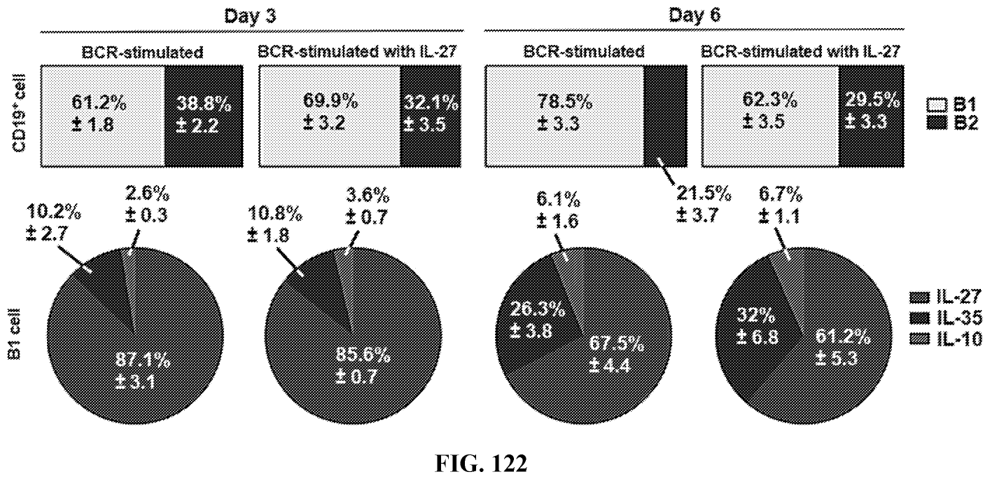

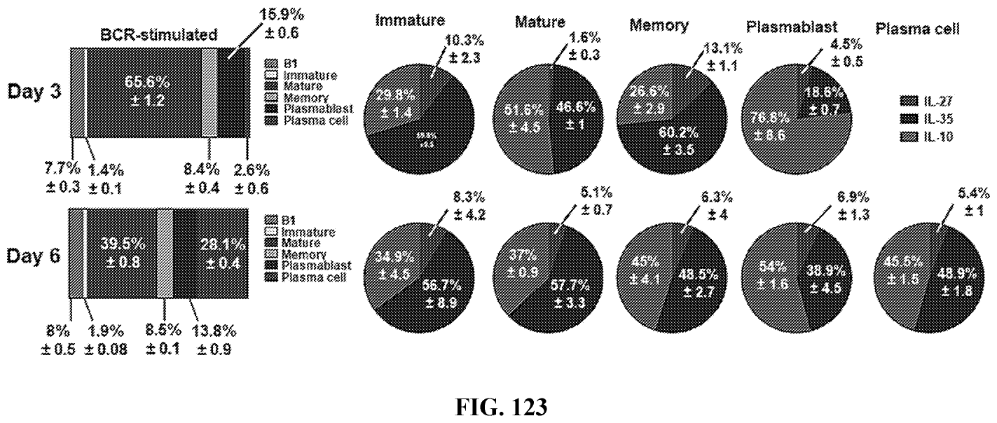

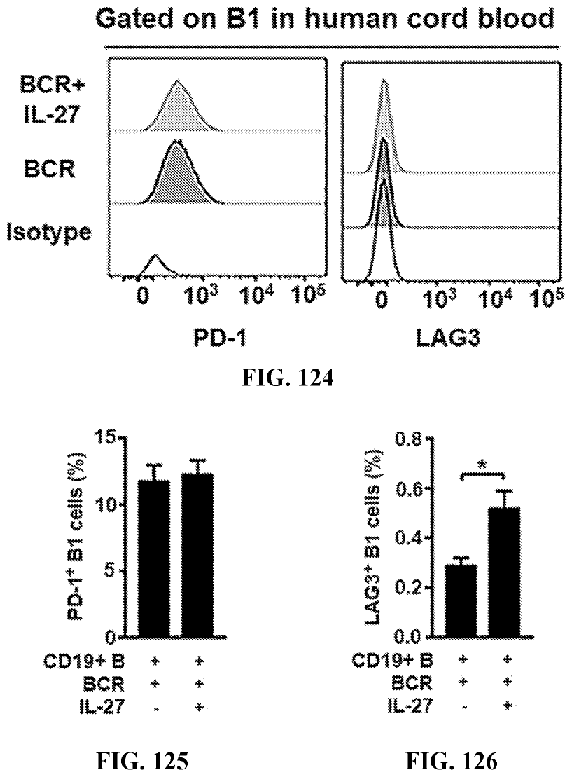

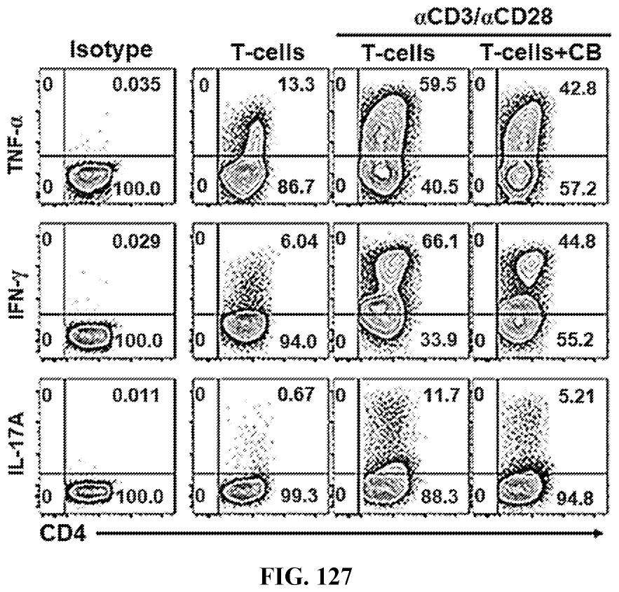

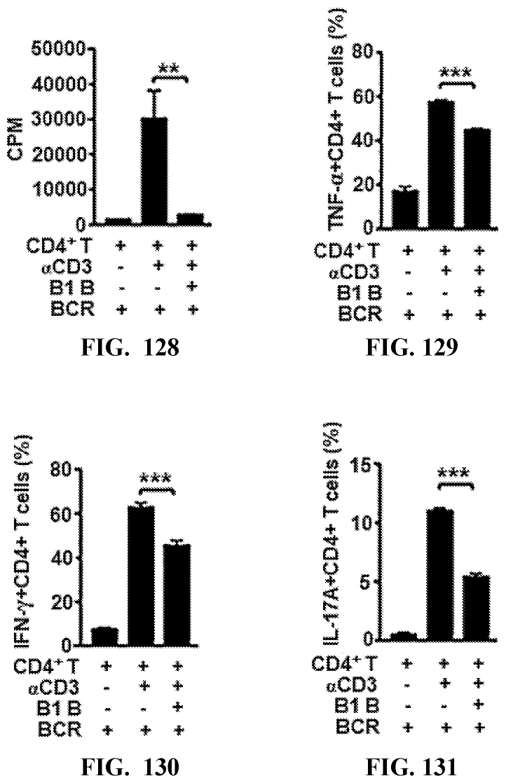

This study examined whether i27-Breg cells existed in humans and expanded in response to inflammatory stimuli by culturing healthy human PBMC for 3 days with TLR agonist CpG and BCR (anti-CD40 or anti-IgM). Gating on human B-1 cells (CD19⁺CD20⁺CD27⁺CD43⁺) revealed that as high as 19.9% of BCR-activated B-cells in-human PBMC produced IL-27 (FIGS. 114 and 115). CD19⁺CD20+CD27+CD43+CD11⁺B-1 cells represent a subset of B-1a cells that are developmentally poised to migrate to the spleen and other sites of antibody production in response to appropriate stimuli and gating on this cell population revealed that as many as 35% of BCR-activated human B-1a cells (FIGS. 116-118) can be recruited into the spleen and inflammatory sites during inflammatory diseases. Analysis of human umbilical cord blood from healthy human donors revealed that as much as 18.1% of resting B-1a cells constitutively produce IL-27 and stimulation of BCR-activated cord blood B-cells with IL-27 increased percentage of cord blood i27-Bregs to 73.9% (FIGS. 119-121). To determine the relative abundance of i27-Bregs viz-a-viz other Breg subtypes (IL-10-producing Bregs and i35-Bregs), activated cord blood cells were propagated for 6 days. While the majority of the Breg cells were i27-Bregs, low levels of IL-10-producing Bregs and i35-Bregs were detected, and their levels increased in a time dependent manner (FIG. 122). Similar analysis of B-2 cells revealed that most i27-Bregs were either in the naïve or memory B-cell pool (FIG. 123). Similar to the mouse species, the human i27-Breg cells constitutively express inhibitory receptors PD-1 and LAG3 (FIGS. 124-126) and suppressed proliferative responses of TNF-α-, IL-17-, and/or IFN-γ-producing pro-inflammatory CD4⁺T-cells (FIGS. 127-131). The enrichment of i27-Bregs in cord blood is of clinical interest because cord blood is the preferred source of hematopoietic stem cells for allogeneic (non-self) transplantation for patients with significant miss-matched human leukocyte antigen (HLA; a gene complex encoding the major histocompatibility complex (MHC) proteins in humans), and cord blood i27-Bregs can therefore be exploited to suppress alloreactive responses and protect against GVHD after allogeneic hematopoietic transplantation.

### EXAMPLE 8

This example demonstrates that human i27-Breg cells can be used to successfully treat humans suffering from a disease or at risk for suffering from a disease.

Human i27-Breg cells are administered, by injection or i.v., to a human suffering from a disease, such as uveitis, MS, AMD, and/or GVHD, or a human in need of the prevention of a disease, such as GVHD. Following administration of the human i27-Breg cells, the severity and/or the symptoms of the disease will be decreased and/or prevented.

### EXAMPLE 9

This example demonstrates that i27-Breg have a unique transcriptome.

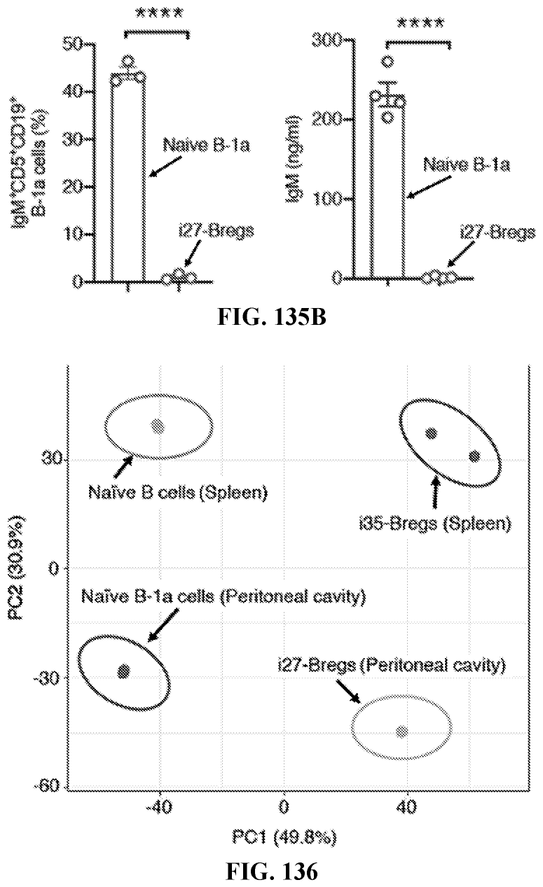

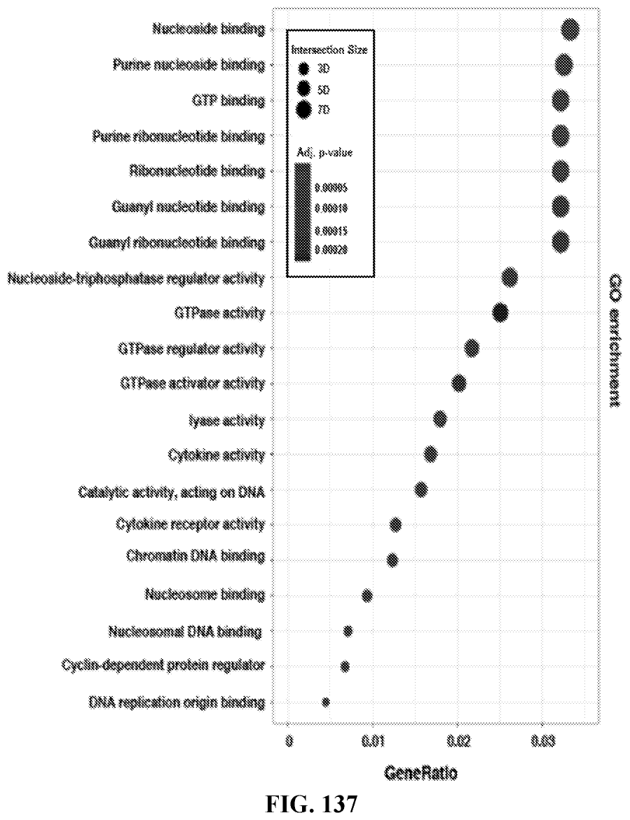

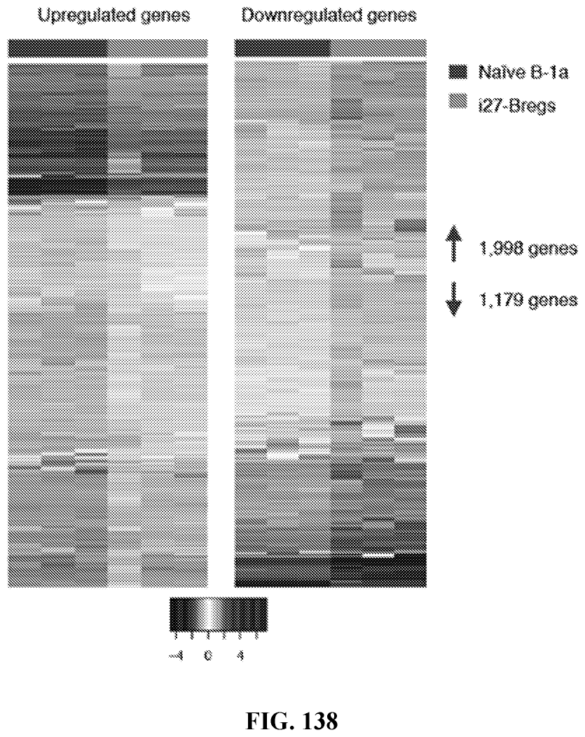

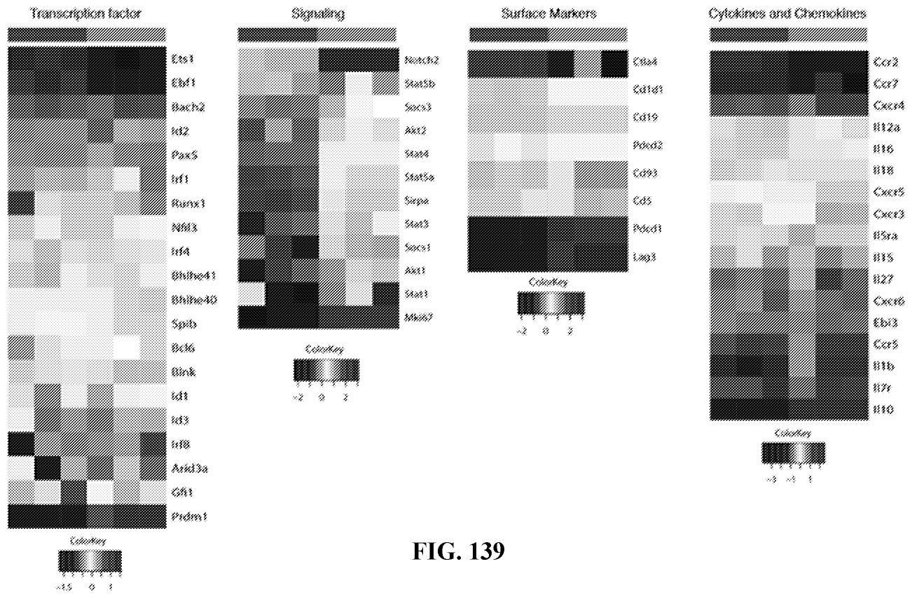

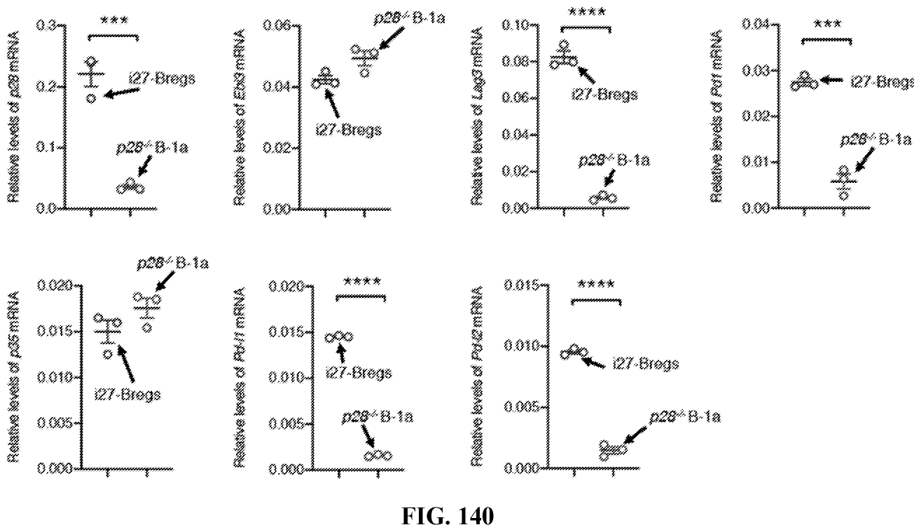

Peritoneal cavity B-1a cells enriched for i27-Breg cell by activation with BCR and IL-27 were used to determine the gene expression program required for the development of i27-Breg cells. Characterization of the highly enriched IL-27-producing B-1a cells (>83% i27-Bregs) revealed that while B-1a cells constitutively secrete natural IgM antibodies, the development into i27-Breg cell phenotype coincides with loss of capacity to produce IgM (FIGS. 135A-135B). Besides the unchallenged B-1a cells, conventional B-2 and IL-35-producing B-2 cells (>57% i35-Breg) from mouse spleen were used as comparators for RNA-seq analysis. Principal component analysis (PCA) of differentially regulated genes clearly separated the B cells into 4 distinct populations (FIG. 136). Gene ontology (GO) analysis identified highly enriched genes encoding proteins that enhance molecular processes and pathways that further characterize the unique immune-suppressive activities of i27-Breg cells (FIG. 137). Heatmaps derived from global RNA-Seq analysis identified 1,998 genes that were unregulated in i27-Breg and 1,179 genes that were downregulated (FIG. 137). Genes differentially induced in i27-Breg (>2-fold higher expression) included those that encode cytokines, cytokine receptors and chemokine receptors (1127, Ebi3, I110, I17r, I121r, Cxcr3, Cxcr5), inhibitory receptors (Pdcdl, Lag3), signaling molecules (Notch4, Statl, Stat3, StatS, Aktl, Akt2), transcription factors (Irf8, Irfl, Batf, Bhlhe40, Xbpl, Arid3a, Ikzfl, Ikzf2, Ikzf4). Repressed genes included 1112a, Notch2 Cxcr4, Ccr2, Ccr7), genes that encode inhibitory receptors (Pdcd2, Cdldl, Ctla4) and transcription factors (Irf4, Ikzf3, Bach2, Pax5, Ebfl, Runxl, Foxol, Etsl) (FIG. 139). To further validate that IL-27 is required for maintaining the i27-Breg transcriptome we show that IL-27 deficient B-1a cells express IL-35 (p35 and EBi3) but are defective in expressing inhibitory receptors genes (Lag3, Pdl, as well as Pd-11, Pd-12) (FIG. 140). Taken together, these results suggest that i27-Breg transcriptome exhibits significant increase of genes (Bhlhe40, Arid3a, and Cd5) required for B-1a development, underscoring the developmental origin of i27-Breg from innate B-1 cells. However, the i27-Breg cell also exhibits transcription signature characteristic of differentiating germinal center B cells (Irf8T, BatfT, Pax5T, Bach2T, EbflT) but not of terminally differentiated plasma cells (PrdmlT, Bach2T, Pax5T, EbflT), demonstrating that i27-Breg has a unique transcriptome.

### EXAMPLE 10

This example demonstrates that i27-Breg and i35-Breg in human cord blood and PBMC have distinct transcriptomic profiles.

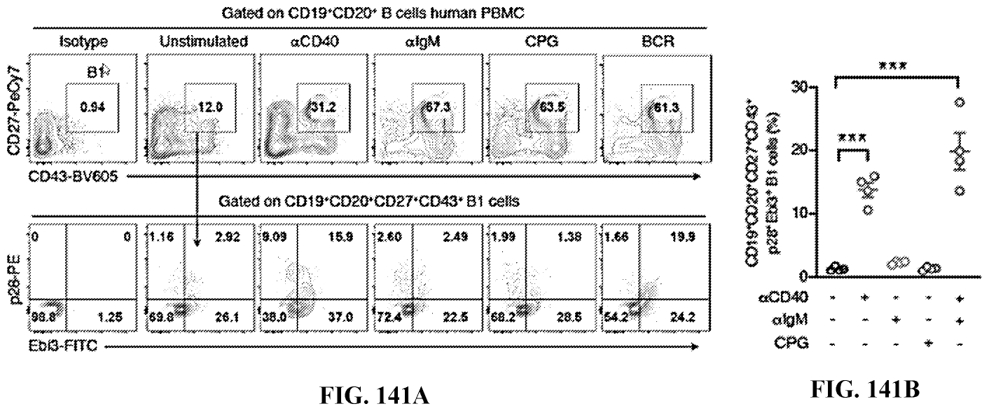

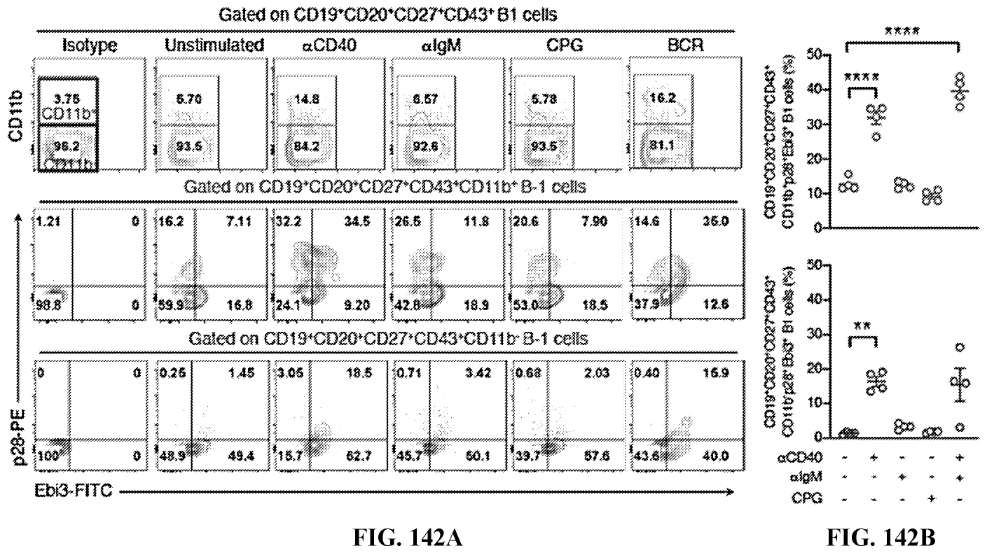

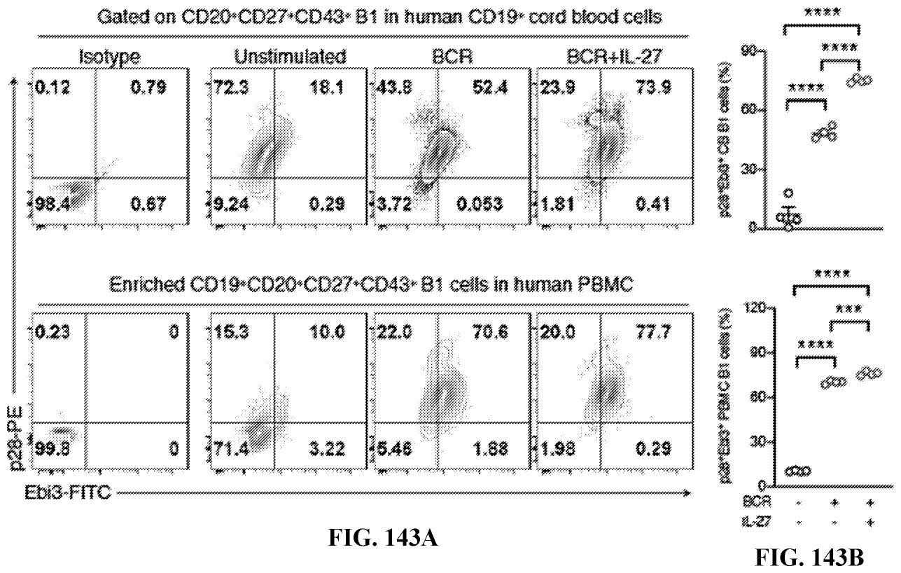

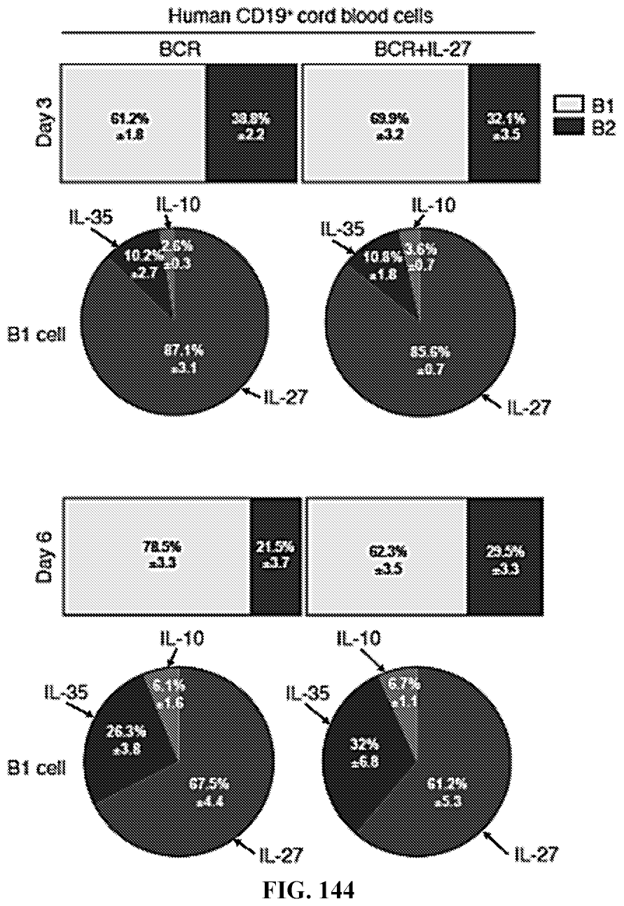

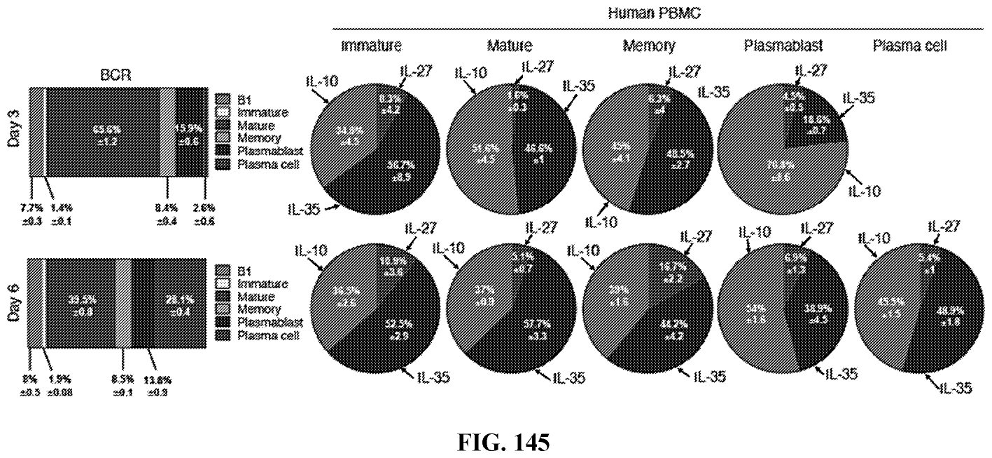

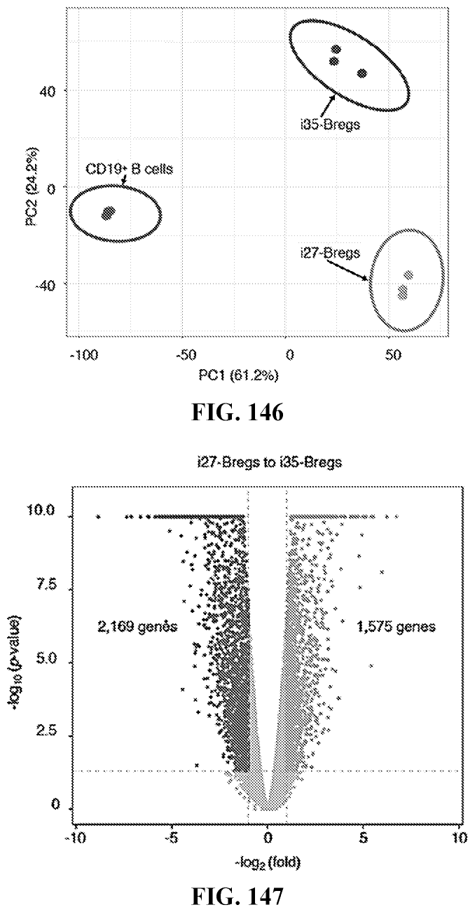

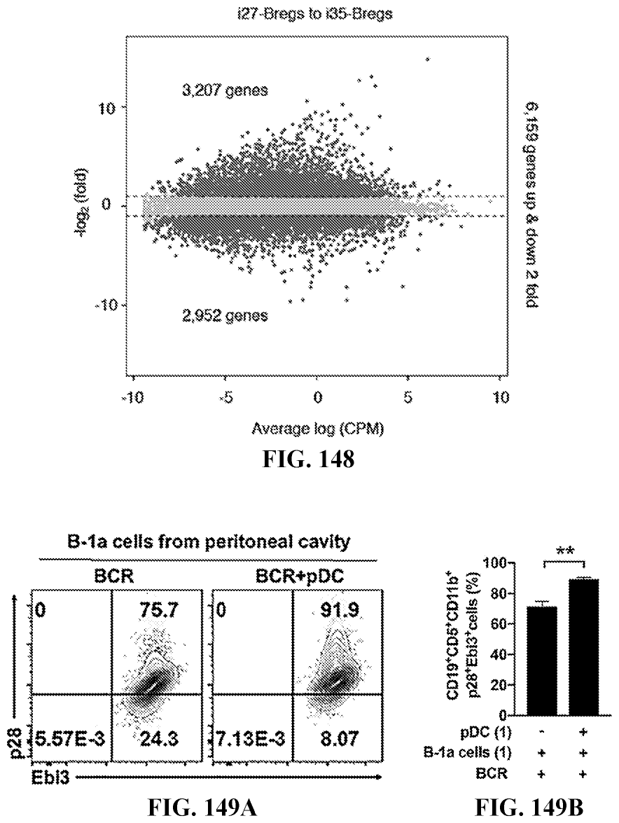

Human PBMC and cord blood (CB) B cells produced IL-27 and in the PBMC ˜19.9% of activated B-1-like cells (CD19⁺CD2⁰⁺CD27⁺CD43⁺) are i27-bregs (FIG. 141A and 141B). More than 40% of the i27-Breg cells exhibited the CD19⁺CD20⁺CD27⁺CD43⁺CD11⁺ phenotype (FIG. 142A-142C), a B-1a subset in body cavities known to redistribute to regional lymph node in response to inflammation. On the other hand, ˜18.1% of resting B-1a cells in CB constitutively secreted IL-27 and upon activation in presence of IL-27, the percentage of CB i27-Bregs dramatically increased to 73.9% (FIG. 143A-143C), suggesting that i27-Bregs may serve as natural Bregs in human CB, poised for rapid mobilization to regional lymph nodes in response to inflammation. t-SNE clustering analysis grouped Breg cells in the CB into 3 distinct spatially segregated subsets: B10, i27-Breg and i35-Breg; i27-Bregs were the most abundant, comprising >85% of the Bregs in day 3 cultures and declining to less than 61% in the day 6 cultures (FIG. 144). Although B10 and i35-Breg cells were relatively sparse in day 3 cultures, i35-Bregs increased substantially (32%) by Day 6 (FIG. 144). Interestingly, B cells at all stages of development were capable of producing IL-10, IL-27, or IL-35 although i27-Bregs were most abundant in immature and memory B cells (FIG. 145). Principal component and RNA-seq analyses revealed that i27-Breg and i35-Breg have distinct transcriptomic profiles (FIG. 146); of the 3,744 differentially expressed genes, 1,575 were elevated in i27-Bregs while 2,169 were downregulated (FIG. 147). Similar comparison between CD19⁺ B cells and i27-Bregs found that of the 6,159 genes differentially expressed, 3,207 were unregulated in i27-Breg (FIG. 148). Results of analysis of human PBMC or CB thus suggest that different Breg subsets are induced during the course of an inflammatory response and the relative abundance of each subset fluctuates depending on the nature of the inflammatory challenge.

### EXAMPLE 11

This example demonstrates that innate i27-Bregs suppress CNS autoimmune disease through a BCR-independent mechanism.

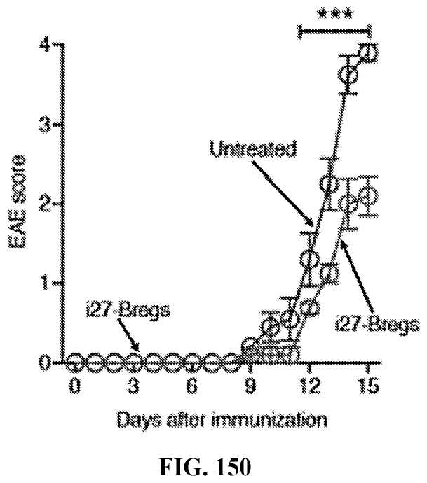

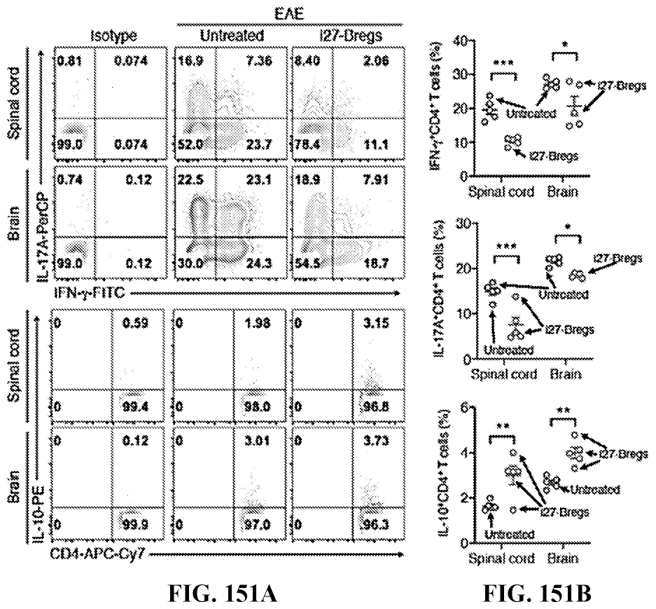

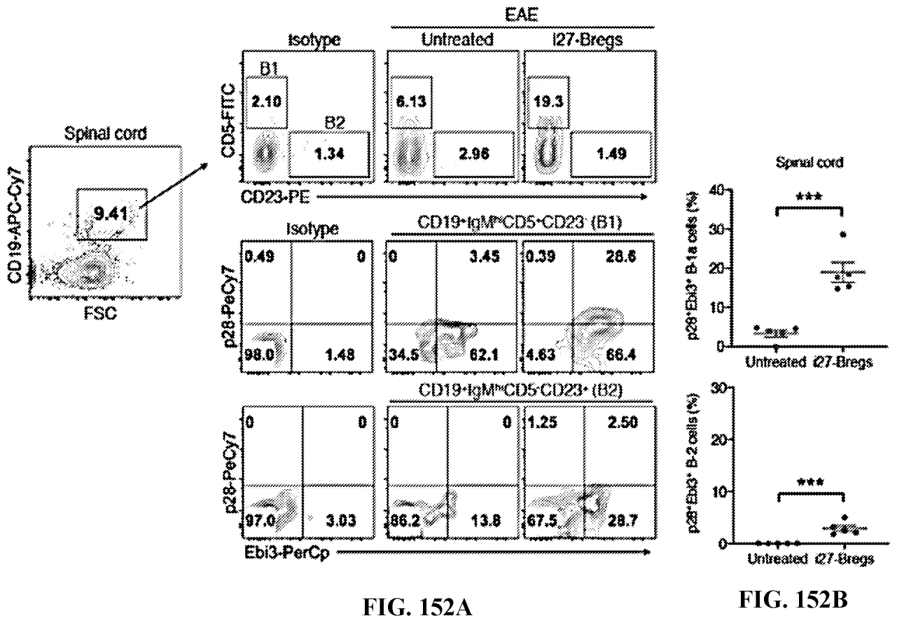

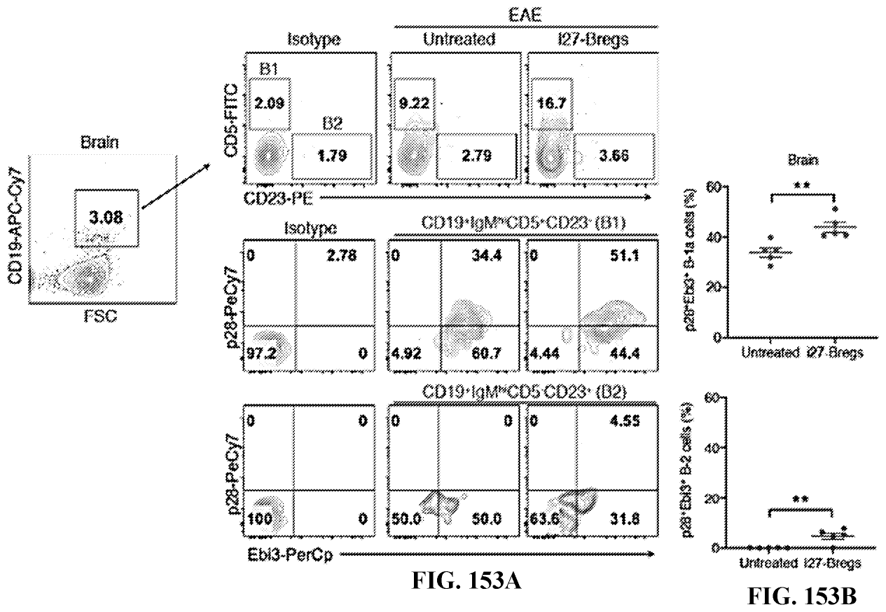

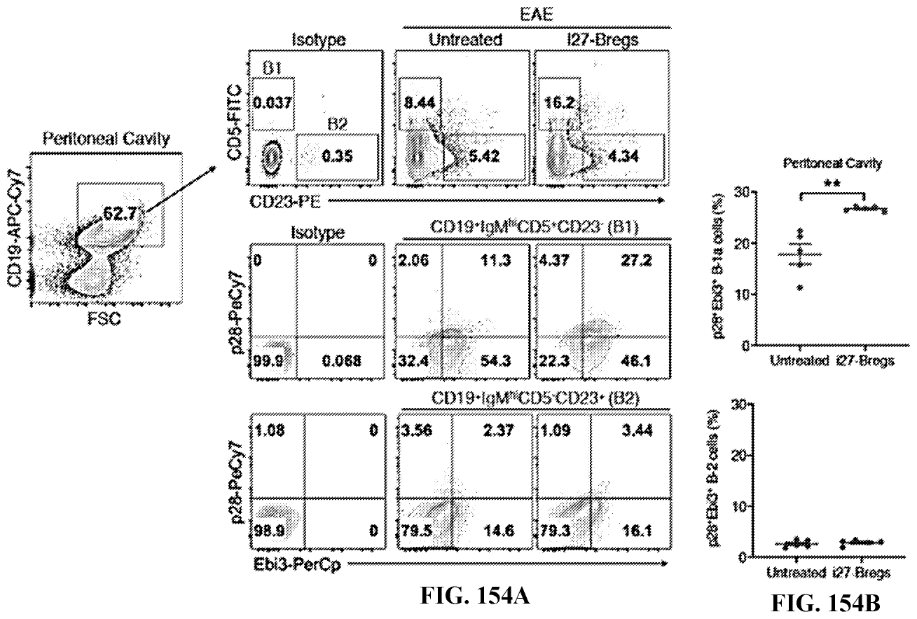

Peritoneal cavity B-1 cells are to a large extent unresponsive to BCR-induced signals, but highly responsive to innate immune signals induced by pathogens and TLR agonists, suggesting that immune-suppressive activities of i27-Bregs. To address whether i27-Breg-mediated suppression of EAU or EAE requires prior activation by IRBP or MOG autoantigen, “sepsis” was induced in CD45.2⁺C57BL/6J mice by injection of LPS, sorted B-1a cells (>83.5% i27-Bregs) from the peritoneal cavity, and the i27-Breg-enriched cells (5×10⁵/mouse) were adoptively transferred into naïve CD45.1⁺congenic mice. 24 h later the mice were challenged by EAE induction. Clinical evaluation of the mice revealed significant suppression of EAE (FIG. 150) or EAU, compared to control mice that received equivalent number of B-1a (<7% i27-Breg) cells. Disease amelioration correlated with reduction of IL-17-single positive and IL-17/IFN-γ-double positive Th17 cells and expansion of Tregs in brain and spinal cord (FIGS. 151A-151B); expansion of B-1a i27-Breg cells in spinal cord (FIG. 152A-152B), brain (FIGS. 153A-153B) and peritoneal cavity (FIGS. 154A-154B). These results support that adoptive i27-Breg therapy can be useful as a treatment for autoimmune diseases.

Collectively, the above examples show an innate IL-27-producing Breg population exists in human cord blood, PBMC, as well as the brain, spinal cord, retina, and peritoneal cavity of mice suffering from experimental autoimmune encephalomyelitis (EAE) or experimental autoimmune uveitis (EAU), which are models of multiple sclerosis and uveitis, respectively. In vitro experimental systems including, confocal microscopy, FACS-based cell sorting, RNA-seq, Chip assay, and immunohistochemistry show that the IL-27-producing Breg has a unique transcriptome and is functionally distinct from other Bregs. Adoptive transfer of i27-Bregs ameliorated EAE and EAU by reprogramming resting B cells to i35-Breg cells that trafficked to the uvea, brain, and spinal cord and suppressed pathogenic T cells, thus demonstrating the efficacy of i27-Breg immunotherapy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated population of mammal cells comprising about 75% or higher B-1a regulatory cells:
(a) expressing cell surface inhibitory receptors lymphocyte-activation gene 3 (LAG-3), programmed cell death protein 1 (PD-1), and C-X-C chemokine receptor type 4 (CXCR4); and
(b) secreting interleukin-27 (IL-27).

2. The population of mammal cells of claim 1, wherein the regulatory cells further express cell surface inhibitory receptor glucocorticoid-induced TNFR-related protein (GITR).

3. The population of mammal cells of claim 1, wherein the regulatory cells further express cell surface inhibitory receptor OX40.

4. The population of mammal cells of claim 1, wherein the regulatory cells further express cell surface inhibitory receptor cytotoxic T-lymphocyte-associated protein 4 (CTLA4).

5. A method of preparing the population of mammal cells of claim 1, comprising
(a) isolating cluster of differentiation 5 positive (CD5+) expressing cells from a sample of mammal peripheral lymphoid tissue, mammal cord blood, mammal peritoneal fluid, induced pluripotent cells (iPSC), or mammal bone marrow using fluorescence-activated cell sorting (FACS) to provide isolated CD5+ expressing cells;
(b) culturing the isolated CD5+ expressing cells in a cell culture media to provide cultured cells;
(c) activating the cultured cells with a BCR (B cell receptor) or a TLR (Toll-like receptor) agonists to provide activated cells; and
(d) exposing the activated cells to IL-27.

6. A method of suppressing the immune system in a mammal, the method comprising administering to a mammal the population of mammal cells of claim 1.

7. The method of claim 6, further comprising sequentially or simultaneously administering B-cells that produce interleukin-35 (IL-35) to the mammal.

8. The method of claim 6, wherein administration treats a disease in the mammal.

9. The method of claim 6, wherein the mammal has an autoimmune disease.

10. The method of claim 9, wherein the autoimmune disease is a disease of the eye, disease of the central nervous system, disease of the brain, uveitis, or encephalomyelitis.

11-14. (canceled)

15. The method of claim 6, wherein the mammal has multiple sclerosis.

16. The method of claim 6, wherein administration suppresses inflammation of the pancreas.

17. The method of claim 6, wherein the mammal has received an allogeneic bone marrow, marrow or hematopoietic stem cell transplant, or allogeneic solid organ transplant.

18. (canceled)

19. The method of claim 17, wherein the mammal has graft-versus-host disease (GVHD) or age-related macular degeneration (AMD).

20. (canceled)

21. A method of treating a mammal with graft-versus-host disease, the method comprising administering the population of mammal cells of claim 1 to a mammal with graft-versus-host disease.

22. The method of claim 21, wherein the mammal received an allogeneic bone marrow, hematopoietic stem cell transplant, or an allogeneic solid organ transplant prior to the administration of the population of mammal cells.

23. (canceled)

24. A method preventing or reducing the severity of graft-versus-host disease in a mammal, the method comprising administering the population of mammal cells of claim 1 to a mammal before the mammal receives an allogeneic transplant.

25. The method of claim 24, wherein the allogeneic transplant is an allogeneic bone marrow, hematopoietic stem cell transplant, or an allogeneic solid organ transplant.

26-27. (canceled)

28. A method of preventing or reducing the severity of graft-versus-host disease in a mammal, the method comprising
(a) mixing the population of mammal cells of claim 1 with a transplant material to form a transplant mixture; and
(b) administering the transplant mixture to a mammal.

29. The population of mammal cells of claim 1, wherein the mammal is a human.


SEQUENCE LISTING (13 sequences)

SEQ ID NO: 1 - DNA, 19 bp, Artificial Sequence
  synthetic
gcttcctcgctaccacact

SEQ ID NO: 2 - DNA, 20 bp, Artificial Sequence
  synthetic
gggccatgaggctggatctc

SEQ ID NO: 3 - DNA, 20 bp, Artificial Sequence
  synthetic
gatggtatcccaggggcagg

SEQ ID NO: 4 - DNA, 20 bp, Artificial Sequence
  synthetic
gtcggggatggtgcatcggg

SEQ ID NO: 5 - DNA, 20 bp, Artificial Sequence
  synthetic
tctctgatgggtcactaact

SEQ ID NO: 6 - DNA, 20 bp, Artificial Sequence
  synthetic
caggagcagtccacggccac

SEQ ID NO: 7 - DNA, 22 bp, Artificial Sequence
  synthetic
ctgaaaccccagcttcctgcca

SEQ ID NO: 8 - DNA, 28 bp, Artificial Sequence
  synthetic
catctcctgggtaggggggtcttatact

SEQ ID NO: 9 - DNA, 17 bp, Artificial Sequence
  synthetic
ggaagggaaattacgtt

SEQ ID NO: 10 - DNA, 27 bp, Artificial Sequence
  synthetic
ctgattctgtctctgtttctctcagtt

SEQ ID NO: 11 - DNA, 23 bp, Artificial Sequence
  synthetic
gtggggaaaggccttgaggtaga

SEQ ID NO: 12 - DNA, 15 bp, Artificial Sequence
  synthetic
cctcaaggcctttcc

SEQ ID NO: 13 - DNA, 4 bp, Artificial Sequence
  synthetic
gaaa